(19) United States
(12) Patent Application Publication
(10) Pub. No.: US 2025/0323417 A1
(43) Pub. Date: Oct. 16, 2025

(54) PHYSIOLOGICAL MONITORING DEVICES, SYSTEMS, AND METHODS FOR DATA INTEGRATION

(71) Applicant: Masimo Corporation, Irvine, CA (US)

(72) Inventors: Eduardo Rey, Dana Point, CA (US); Joani Bajlozi, Aliso Viejo, CA (US); Ammar Al-Ali, San Juan Capistrano, CA (US); Stephen Scruggs, Newport Beach, CA (US); Chad A. DeJong, Los Angeles, CA (US)

(21) Appl. No.: 19/174,799

(22) Filed: Apr. 9, 2025

Related U.S. Application Data
(60) Provisional application No. 63/633,237, filed on Apr. 12, 2024.
(60) Provisional application No. 63/659,262, filed on Jun. 12, 2024.
(60) Provisional application No. 63/713,426, filed on Oct. 29, 2024.
(60) Provisional application No. 63/714,714, filed on Oct. 31, 2024.
(60) Provisional application No. 63/777,987, filed on Mar. 26, 2025.

(51) Int. Cl. H01Q 5/48; H01Q 1/27; H01Q 5/307

(52) U.S. Cl. CPC H01Q 5/48; H01Q 1/273; H01Q 5/307

(57) ABSTRACT

An electronic device can be configured to be secured to a subject and measure one or more physiological parameters of the subject. The electronic device may include a housing, including a bottom portion, a top portion, a first side connected to the top portion along a first edge, a second side connected to the top portion along a second edge, a third side connected to the top portion along a third edge, a fourth side connected to the top portion along a fourth edge. The electronic device may include one or more physiological sensors for measuring the one or more physiological parameters. The electronic device may include an antenna configured to allow the electronic device to wirelessly communicate with one or more separate devices. The antenna may extend along an interior surface at least partially along each of the first edge, second edge, third edge, and fourth edge.

### INCORPORATION BY REFERENCE TO ANY PRIORITY APPLICATIONS

The present application claims priority to U.S. Provisional Application No. 63/633,237, filed Apr. 12, 2024, U.S. Provisional Application No. 63/659,262, filed Jun. 12, 2024, U.S. Provisional Application No. 63/713,426, filed Oct. 29, 2024, U.S. Provisional Application No. 63/714,714, filed Oct. 31, 2024, and U.S. Provisional Application No. 63/777,987, filed Mar. 26, 2025. All of the above-listed applications and any and all other applications for which a foreign or domestic priority claim is identified in the Application Data Sheet as filed with the present application, are hereby incorporated by reference under 37 CFR 1.57.

### TECHNICAL FIELD

The present disclosure relates to physiological monitoring devices, systems, and methods.

### BACKGROUND

Hospitals, nursing homes, and other patient care facilities typically utilize a number of sensors, devices, and/or monitors to collect or analyze a patient's physiological parameters. Various conventional sensor systems exist which collect physiological data using physiological sensors, process the data, and display the data on a display device. Clinicians, including doctors, nurses, and other medical personnel, use the physiological parameters obtained from patient monitors to diagnose illnesses and to prescribe treatments. Clinicians also use the physiological parameters to monitor patients during various clinical situations to determine whether to increase the level of medical care given to patients.

### SUMMARY

Some conventional sensor systems require time-consuming and complex procedures for changing the monitoring of physiological data between display devices. For example, a user may be required to unplug sensors from a first display device and replug the sensors into a second display device. As another example, a user may be required to power the sensors off and then power them on to wirelessly connect with a different display device. As another example, a user may be required to manually change a pairing status of the sensors and/or display devices to terminate a wireless connection with one display device and/or to establish a wireless connection with another display device. For at least the foregoing examples, changing monitoring of physiological data from a first display device to a second display device in a conventional sensor system can take long amounts of time, can be overly complex and frustrating to a user, can result in user error, and can result in loss of physiological data such as during a time in which the sensors are not connected (for example, wirelessly and/or wired) to any display device.

Furthermore, some conventional sensor systems require time-consuming and complex procedures for initiating physiological monitoring of a patient, for example, when intaking a patient in a healthcare environment. Such initiation can include associating a physiological monitoring system, such as devices of the monitoring system and/or devices in communication with the monitoring system, with the patient. For example, a user (or caregiver) may be required to manually enter patient identification data into a physiological monitoring system or devices thereof in order to associate physiological data measured by the system with the patient. Such entry by a user can be time consuming, overly complex, frustrating to a user, result in user error, and/or result in loss of physiological data. Additionally, some conventional sensor systems lack the capability or require time-consuming and complex procedures for associating historical physiological data of a patient (such as physiological data of the patient measured before entering the healthcare environment) with the patient. The systems described herein can advantageously save time and reduce errors by automatically associating physiological data measured thereby with patient identification data. The systems described herein can advantageously improve a level of care provided to the patient by having the capability to associate historical physiological data of the patient with the patient and any physiological data measured by the system after initiation (for example, after intake in the healthcare environment).

Disclosed herein is a computing system configured to facilitate monitoring a patient when the patient transitions between environments. The computing system can comprise: an in-room display terminal associated with a healthcare environment and configured to display indicia of a health of the patient for electronically monitoring the health of the patient within the healthcare environment; and one or more hardware computer processors configured to execute program instructions to cause the computing system to: receive, via the in-room display terminal, a request to initiate monitoring the patient with the in-room display terminal at the healthcare environment; responsive to the request, access historical physiological data associated with the patient and generated by a home monitoring device before the patient enters the healthcare environment; access real-time physiological data associated with the patient and originating from a physiological monitoring device coupled to the patient within the healthcare environment; and responsive to determining that the historical physiological data originates from an approved device: generate one or more physiological parameters from the real-time physiological data and the historical physiological data; and cause the in-room display terminal to display indicia of the one or more physiological parameters.

In the above computing system or in other implementations as described herein, one or more of the following features can also be provided. In some implementations, the one or more hardware computer processors are configured to execute the program instructions to cause the computing system to: responsive to determining that at least a portion of the historical physiological data originates from an unapproved device, apply weights to the historical physiological data to generate weighted historical physiological data based on whether the historical physiological data originates from an approved device or an unapproved device; and generate one or more weighted physiological parameters from the real-time physiological data and the weighted historical physiological data to be displayed at the in-room display terminal. In some implementations, the one or more hardware computer processors are configured to execute the program instructions to cause the computing system to: update metadata of the historical physiological data to indicate whether the historical physiological data originates from an approved device. In some implementations, the one or more hardware computer processors are configured to execute the program instructions to cause the computing system to: cause the in-room display terminal to display indicia of whether the historical physiological data originates from an approved device. In some implementations, the one or more hardware computer processors are configured to execute the program instructions to cause the computing system to: receive the historical physiological data from a server; and determine whether the historical physiological data originates from an approved device based on at least the server from which the historical physiological data is received. In some implementations, the one or more hardware computer processors are configured to execute the program instructions to cause the computing system to: receive the request to initiate monitoring the patient with the in-room display terminal responsive to NFC communication between the in-room display terminal and a user device and/or the home monitoring device. In some implementations, the one or more hardware computer processors are configured to execute the program instructions to cause the computing system to: receive the request to initiate monitoring the patient with the in-room display terminal responsive to authorization via a user device and/or the home monitoring device. In some implementations, the one or more hardware computer processors are configured to execute the program instructions to cause the computing system to: access a subset of the historical physiological data based on a health condition identified in the historical physiological data. In some implementations, the one or more hardware computer processors are configured to execute the program instructions to cause the computing system to: receive the real-time physiological data from the in-room display terminal. In some implementations, the one or more hardware computer processors are configured to execute the program instructions to cause the computing system to: receive the historical physiological data associated with the patient from one or more of the home monitoring device or a server. In some implementations, the one or more hardware computer processors are configured to execute the program instructions to cause the computing system to: parse the historical physiological data into a structured data format corresponding to a format of the real-time physiological data. In some implementations, the one or more hardware computer processors are configured to execute the program instructions to cause the computing system to: generate user interface data for rendering indicia of the real-time physiological data originating from the physiological monitoring device in combination with the historical physiological data generated by the home monitoring device.

Disclosed herein is a method of monitoring a patient when the patient transitions between environments. The method can comprise: receiving, via an in-room display terminal associated with a healthcare environment, a request to initiate monitoring the patient with the in-room display terminal at the healthcare environment; responsive to the request, accessing historical physiological data associated with the patient and generated by a home monitoring device before the patient enters the healthcare environment; accessing real-time physiological data associated with the patient and originating from a physiological monitoring device coupled to the patient within the healthcare environment; and responsive to determining that the historical physiological data originates from an approved device: generating one or more physiological parameters from the real-time physiological data and the historical physiological data; and causing the in-room display terminal to display indicia of the one or more physiological parameters.

In the above method or in other implementations as described herein, one or more of the following features can also be provided. In some implementations, the method further comprises: responsive to determining that at least a portion of the historical physiological data originates from an unapproved device, applying weights to the historical physiological data to generate weighted historical physiological data based on whether the historical physiological data originates from an approved device or an unapproved device; and generating one or more weighted physiological parameters from the real-time physiological data and the weighted historical physiological data to be displayed at the in-room display terminal. In some implementations, the method further comprises: updating metadata of the historical physiological data to indicate whether the historical physiological data originates from an approved device. In some implementations, the method further comprises: causing the in-room display terminal to display indicia of whether the historical physiological data originates from an approved device.

Disclosed herein is non-transitory computer-readable media including computer-executable instructions that, when executed by a computing system, cause the computing system to perform operations that can comprise: receiving, via an in-room display terminal associated with a healthcare environment, a request to initiate monitoring a patient with the in-room display terminal at the healthcare environment; responsive to the request, accessing historical physiological data associated with the patient and generated by a home monitoring device before the patient enters the healthcare environment; accessing real-time physiological data associated with the patient and originating from a physiological monitoring device coupled to the patient within the healthcare environment; and responsive to determining that the historical physiological data originates from an approved device: generating one or more physiological parameters from the real-time physiological data and the historical physiological data; and causing the in-room display terminal to display indicia of the one or more physiological parameters.

In the above non-transitory computer-readable media or in other implementations as described herein, one or more of the following features can also be provided. In some implementations, the computer-executable instructions, when executed by the computing system, cause the computing system to perform operations comprising: responsive to determining that at least a portion of the historical physiological data originates from an unapproved device, applying weights to the historical physiological data to generate weighted historical physiological data based on whether the historical physiological data originates from an approved device or an unapproved device; and generating one or more weighted physiological parameters from the real-time physiological data and the weighted historical physiological data to be displayed at the in-room display terminal. In some implementations, the computer-executable instructions, when executed by the computing system, cause the computing system to perform operations comprising: updating metadata of the historical physiological data to indicate whether the historical physiological data originates from an approved device. In some implementations, the computer-executable instructions, when executed by the computing system, cause the computing system to perform operations comprising: causing the in-room display terminal to display indicia of whether the historical physiological data originates from an approved device.

Some of the systems described herein utilize pulse oximetry sensor(s) for determination of a variety of physiological parameters and/or characteristics, including but not limited to oxygen saturation (SpO2), pulse rate, a plethysmograph waveform, perfusion index (PI), pleth variability index (PVI), methemoglobin (MetHb), carboxyhemoglobin (CoHb), total hemoglobin (tHb), glucose, and/or otherwise, and the pulse oximetry sensor(s) can be utilized for display on one or more electronic devices the foregoing parameters and/or characteristics individually, in groups, in trends, as combinations, or as an overall wellness or other index. The present disclosure describes various implementations for wearable systems which secure to a subject (for example, to a wrist, a lower arm, an upper arm, and/or an upper body of the subject) and employ pulse oximetry at a wrist, a lower arm, and/or an upper arm of the subject.

Some implementations of the wearable systems disclosed herein include a wearable device configured to be secured to the subject and operably position an electronic device configured to measure at least a pulse oximetry measurement of the subject. The wearable devices described herein can include a main body configured to operably position the electronic device and a securement portion connected to the main body configured to secure the main body to the subject. In some implementations, the securement portion includes a strap, a band, or a garment. Furthermore, the wearable devices described herein can include a storage component that can store patient identification data. Such patient identification data can advantageously be transmitted (for example, automatically) to an electronic device when the electronic device is secured to the wearable device. The electronic device can advantageously associate physiological data measured thereby with the patient identification data, which can save time and reduce errors in the healthcare environment. Additionally, the electronic device can transmit the physiological data associated with the patient identification data to an external device (such as a monitoring hub as described herein).

Some implementations of the disclosed wearable systems (or portions of such systems) can be waterproof, thereby providing minimal disruption to ordinary activities of the user (for example, showering). Some implementations of the disclosed wearable systems include two separable components (which may also be referred to as “separate portions”). In such implementations, a first one of the components can be configured to secure to a portion of a user (for example, skin of the user) and a second one of the components can be configured to secure (for example, removably secure) to the first component. In some implementations, the first and second components are configured such that separation thereof is inhibited or prevented when the first component is secured to the user but is allowed when the first component is not secured to the user. Such implementations can be advantageous in scenarios where it is desirable to inhibit or prevent a user from interfering with operation of the wearable systems. In some implementations, the wearable systems includes a button configured to transition the wearable systems (or a portion thereof such as the second component discussed above) between non-operational and operational modes. In some of such implementations, such button is inaccessible (for example, to the user wearing the wearable systems and/or to another person, such as a care provider) unless the first and second components are separated from one another. Such implementation can advantageously prevent a user (for example, a child) from intentionally or unintentionally turning the wearable systems off when the wearable systems is secured to the user (which can ensure proper compliance in some situations).

Disclosed herein is a wearable system comprising an electronic device and a wearable device. The electronic device can comprise a physiological sensor configured to generate physiological data of a subject. The wearable device can be configured to removably secure to the electronic device and be secured to a subject. The wearable device can comprise a storage component configured to store identification data associated with the subject. The electronic device can be configured to: electronically connect to and access said identification data from the storage component when the electronic device is secured to the wearable device; wirelessly receive, from an at-home monitoring device, physiological data of the subject generated during a first time period before the subject enters a healthcare environment; transmit the identification data to a monitoring hub; generate physiological data of the subject during a second time period, said second time period being after said first time period and when the subject is in the healthcare environment; and transmit the physiological data generated during the first time period and the second time period to the monitoring hub.

In the above system or in other implementations as described herein, one or more of the following features can also be provided. In some implementations, the electronic device is configured to: transmit the physiological data measured during the first time period to the monitoring hub responsive to determining that the at-home monitoring device is a medically approved device. In some implementations, the electronic device is configured to: responsive to determining that the at-home monitoring device is an unapproved device, indicate, with metadata, that the physiological data measured during the first time period originates from an unapproved device.

Disclosed herein is a wearable system comprising an electronic device. The electronic device can comprise a physiological sensor configured to generate physiological data of a subject. The electronic device can be configured to: removably mechanically and electronically couple with a wearable device configured to secure to the subject when the subject is in a healthcare environment; access identification data from a storage component of the wearable device, said identification data being associated with the subject; and transmit, to a monitoring hub, physiological data associated with the identification data. The physiological data can comprise: physiological data generated by the physiological sensor during a first time period when the electronic device is removably mechanically coupled with an at-home wearable device before the subject enters the healthcare environment; and physiological data generated by the physiological sensor during a second time period when the electronic device is coupled with the wearable device when the subject is in the healthcare environment.

Disclosed herein is a wearable system comprising an electronic device and a wearable device. The electronic device can comprise at least one sensor configured to generate physiological data of a subject. The wearable device can be configured to removably secure to the electronic device and be secured to a subject. The wearable device can comprise a storage component configured to store identification data associated with the subject. The electronic device can be configured to: electronically connect to and access said identification data from the storage component when the electronic device is secured to the wearable device; transmit said identification data to a remote server to retrieve historical physiological data from the remote server, said identification data being useable to identify the historical physiological data and verify permission to access the historical physiological data; and receive, from the remote server, the historical physiological data, said historical physiological data being associated with the subject and originating from an at-home monitoring device before the subject enters a healthcare environment.

In the above system or in other implementations as described herein, one or more of the following features can also be provided. In some implementations, the electronic device is configured to: responsive to determining that the at-home monitoring device is a medically approved device, indicate that the historical physiological data originates from a medically-approved device with metadata of the historical physiological data. In some implementations, the electronic device is configured to: responsive to determining that the at-home monitoring device is an unapproved device, indicate that the historical physiological data originates from an unapproved device with metadata of the historical physiological data. In some implementations, the electronic device is configured to: apply weights to the historical physiological data based on whether the historical physiological data originates from a medically approved device or an unapproved device; and generate one or more physiological parameters from the historical physiological data with the weights.

Disclosed herein is a monitoring hub comprising one or more hardware processors that can be configured to: receive identification data originating from a wearable system, wherein said identification data is associated with a subject; transmit said identification data to a remote server to retrieve historical physiological data from the remote server, said identification data being useable to identify the historical physiological data and verify permission to access the historical physiological data; and receive, from the remote server, the historical physiological data, said historical physiological data being associated with the subject and originating from an at-home monitoring device before the subject enters a healthcare environment.

In the above monitoring hub or in other implementations as described herein, one or more of the following features can also be provided. In some implementations, the one or more hardware processors are configured to wirelessly receive said identification data from an electronic device of the wearable system that is removably coupled with a wearable device of the wearable system. In some implementations, the one or more hardware processors are configured to: wirelessly receive real-time physiological data from an electronic device of the wearable system that is removably coupled with a wearable device of the wearable system; generate one or more physiological parameters from the real-time physiological data and the historical physiological data; and display indicia of the one or more physiological parameters via a display of the monitoring hub. In some implementations, the one or more hardware processors are configured to: responsive to determining that the at-home monitoring device is a medically approved device, indicate that the historical physiological data originates from a medically-approved device with metadata of the historical physiological data. In some implementations, the one or more hardware processors are configured to: responsive to determining that the at-home monitoring device is an unapproved device, indicate that the historical physiological data originates from an unapproved device with metadata of the historical physiological data. In some implementations, the one or more hardware processors are configured to: apply weights to the historical physiological data based on whether the historical physiological data originates from a medically approved device or an unapproved device; and generate one or more physiological parameters from the historical physiological data with the weights and real-time physiological data originating from the wearable system.

Disclosed herein is a wearable system comprising an electronic device and a wearable device. The electronic device can comprise at least one sensor configured to generate physiological data of a subject. The wearable device can be configured to removably secure to the electronic device and be secured to a subject. The wearable device can comprise a storage component configured to store identification data associated with the subject. The electronic device can be configured to: electronically connect to and access said identification data from the storage component when the electronic device is secured to the wearable device; wirelessly communicate said identification data to a monitoring hub with a request to establish a wireless communication connection with the monitoring hub; and pursuant to establishing the wireless communication connection with the monitoring hub, provide real-time physiological data from the at least one sensor to the monitoring hub.

In the above system or in other implementations as described herein, one or more of the following features can also be provided. In some implementations, the electronic device is configured to: wirelessly communicate said identification data to the monitoring hub to allow the monitoring hub to access historical physiological data from a remote sever with the identification data, the historical physiological data being associated with the identification data. In some implementations, said identification data verifies permission of a user to establish the wireless communication connection.

Disclosed herein is a wearable system comprising an electronic device for measuring one or more physiological parameters of a patient and a wearable device configured to removably secure to the electronic device. The electronic device can comprise: a housing comprising an interior; at least one processor arranged within the interior of the housing; at least one electrical contact in electrical communication with the at least one processor, at least a portion of the at least one electrical contact arranged along an exterior of the housing; a pulse oximetry sensor comprising at least one emitter configured to emit light into tissue of a portion of a body of the patient and at least one detector configured to detect at least a portion of the emitted light after attenuation by said tissue; and a communication component arranged within the interior of the housing and configured for wireless communication with an external device. The wearable device can comprise: at least one strap configured to secure the wearable device and the electronic device to the patient's body; a storage component configured to store patient identification data associated with the patient; and at least one electrical contact in electrical communication with said storage component. When the electronic device and the wearable device are secured to one another, the at least one electrical contact of the wearable device can contact the at least one electrical contact of the electronic device, thereby facilitating transmission of said patient identification data from said wearable device to said electronic device. The electronic device can be configured to wirelessly transmit, via said communication component, physiological data associated with said one or more physiological parameters along with said patient identification data to said external device.

In the above system or in other implementations as described herein, one or more of the following features can also be provided. In some implementations, the wearable device comprises a main body connected to said at least one strap, said main body comprising said storage component and said at least one electrical contact of the wearable device. In some implementations, said main body comprises a frame configured to secure said electronic device. In some implementations, said frame of the main body of the wearable device comprises an opening and said electronic device further comprises a raised portion on a portion of the exterior of the housing, said raised portion configured to be positioned at least partially within said opening when the electronic device and the wearable device are secured to one another. In some implementations, said raised portion is asymmetrically positioned about said exterior of the housing. In some implementations, said main body comprises a frame defining a cavity configured to receive said electronic device. In some implementations, a portion of the at least one electrical contact of the wearable device is arranged along an interior of said cavity. In some implementations, said main body further comprises a retention mechanism connected to a portion of the frame and configured to inhibit removal of the electronic device from said cavity. In some implementations, the electronic device is configured to determine if the wearable device is an authorized product. In some implementations, the electronic device is configured to transmit operational data to the storage component of the wearable device when the at least one electrical contact of the wearable device contacts the at least one electrical contact of the electronic device. In some implementations, said operational data includes a duration of time that the wearable system is in use. In some implementations, the wearable device is configured to become non-operational when said duration of time that the wearable system is in use reaches a threshold value. In some implementations, the at least one strap is configured for single use. In some implementations, the at least one strap is configured to display at least a portion of the patient identification data. In some implementations, the electronic device further comprises a battery. In some implementations, the electronic device further comprises a vibration motor configured to vibrate one or more portions of the wearable system. In some implementations, the electronic device further comprises an audio component configured to produce a sound. In some implementations, the electronic device further comprises an inertial sensor configured to measure a motion and/or a position of the patient and/or the portion of the patient's body. In some implementations, the electronic device further comprises a user input. In some implementations, the electronic device further comprises at least one ECG electrode and is configured to measure at least an ECG measurement. In some implementations, the electronic device further comprises at least one temperature sensor.

Disclosed herein is a wearable system comprising an electronic device comprising at least one sensor for measuring one or more physiological parameters of a subject and a wearable device configured to removably secure to the electronic device and be secured to a portion of the subject's body. The wearable device can comprise a storage component configured to store identification data associated with the subject. The electronic device can be configured to: receive said identification data from the wearable device; and wirelessly transmit physiological data associated with said one or more physiological parameters along with said identification data to an external device.

In the above system or in other implementations as described herein, one or more of the following features can also be provided. In some implementations, the electronic device comprises a communication component configured for wireless communication with the external device. In some implementations, the electronic device is configured to receive said identification data from the wearable device when in proximity thereof. In some implementations, the electronic device is configured to receive said identification data from the wearable device when secured thereto. In some implementations, the electronic device comprises a housing and at least one electrical contact having a portion arranged along an exterior of said housing; the wearable device comprises at least one electrical contact in electrical communication with said storage component; and, when the electronic device and the wearable device are secured to one another, the at least one electrical contact of the wearable device contacts the at least one electrical contact of the electronic device, thereby facilitating transmission of said patient identification data from said wearable device to said electronic device. In some implementations, the wearable device comprises at least one strap configured to secure the wearable device and the electronic device to the portion of the subject's body. In some implementations, the at least one strap is configured for single use. In some implementations, the at least one strap is configured to display at least a portion of the identification data associated with the subject. In some implementations, the wearable device comprises a main body connected to said at least one strap, said main body comprising said storage component. In some implementations, said main body comprises a frame configured to secure said electronic device. In some implementations, said frame of the main body of the wearable device comprises an opening and the electronic device further comprises a raised portion on a portion of an exterior of a housing of the electronic device, said raised portion configured to be positioned at least partially within said opening when the electronic device and the wearable device are secured to one another. In some implementations, said raised portion is asymmetrically positioned about said exterior of the housing. In some implementations, said main body comprises a frame defining a cavity configured to receive said electronic device. In some implementations, said main body further comprises a retention mechanism connected to a portion of the frame and configured to inhibit removal of the electronic device from said cavity. In some implementations, the electronic device is configured to determine if the wearable device is an authorized product. In some implementations, the electronic device is configured to transmit operational data to the storage component of the wearable device when the at least one electrical contact of the wearable device contacts the at least one electrical contact of the electronic device. In some implementations, said operational data includes a duration of time that the wearable system is in use. In some implementations, the wearable device is configured to become non-operational when said duration of time that the wearable system is in use reaches a threshold value. In some implementations, the electronic device further comprises at least one processor and a battery. In some implementations, the electronic device further comprises a pulse oximetry sensor comprising at least one emitter configured to emit light into tissue of the portion of the subject's body and at least one detector configured to detect at least a portion of the emitted light after attenuation by said tissue. In some implementations, the electronic device further comprises a vibration motor configured to vibrate one or more portions of the wearable system. In some implementations, the electronic device further comprises an audio component configured to produce a sound. In some implementations, the electronic device further comprises an inertial sensor configured to measure a motion and/or a position of the subject and/or the portion of the subject's body.

Disclosed herein is a wearable device configured to be secured to a portion of a body of a subject and removably secure to an electronic device comprising one or more physiological sensors, the wearable device comprising a storage component configured to store identification data associated with the subject, the wearable device further configured to provide said identification data to said electronic device.

In the above device or in other implementations as described herein, one or more of the following features can also be provided. In some implementations, the wearable device is configured to provide said identification data to said electronic device when in proximity thereof. In some implementations, the wearable device is configured to provide said identification data to said electronic device when secured thereto. In some implementations, the wearable device is configured to provide said identification data to said electronic device when secured thereto. In some implementations, the wearable device comprises at least one strap configured to secure the wearable device and the electronic device to the portion of the subject's body. In some implementations, the at least one strap is configured for single use. In some implementations, the at least one strap is configured to display at least a portion of the identification data associated with the subject. In some implementations, the wearable device comprises a main body connected to said at least one strap, said main body comprising said storage component. In some implementations, said main body comprises a frame configured to secure said electronic device. In some implementations, said main body comprises a frame defining a cavity configured to receive said electronic device. In some implementations, said main body further comprises a retention mechanism connected to a portion of the frame and configured to inhibit removal of the electronic device from said cavity.

Disclosed herein is a method comprising: obtaining an electronic device comprising at least one sensor for measuring one or more physiological parameters of a patient; obtaining a wearable device configured to be secured to a portion of a body of the patient, the wearable device comprising a storage component configured to store patient identification data associated with the patient; transmitting said patient identification data from said wearable device to said electronic device; and transmitting physiological data associated with said one or more physiological parameters along with said identification data to an external device.

In the above method or in other implementations as described herein, one or more of the following features can also be provided. In some implementations, the method further comprises securing said electronic device to said wearable device. In some implementations, the wearable device comprises a frame configured to secure said electronic device. In some implementations, the wearable device comprises a cavity configured to receive said electronic device. In some implementations, the electronic device comprises at least one electrical contact and the wearable device comprises at least one electrical contact; and wherein when the electronic device is secured to the wearable device, said at least one electrical contact of the electronic device contacts said at least one electrical contact of the wearable device, thereby facilitating said transmitting patient identification data from said wearable device to said electronic device. In some implementations, securing said electronic device to said wearable device comprises bringing the at least one electrical contact of electronic device and the at least one electrical contact of the wearable device into contact with one another. In some implementations, transmitting said patient identification data from said wearable device to said electronic device comprises wirelessly transmitting. In some implementations, transmitting physiological data associated with said one or more physiological parameters along with said identification data to an external device comprises wirelessly transmitting. In some implementations, the method further comprises securing said wearable device to the portion of the body of the patient and measuring, by the electronic device, said one or more physiological parameters of the patient. In some implementations, the method further comprises transmitting said patient identification data to said storage component. In some implementations, the method further comprises determining, by the electronic device, if the wearable device is an authorized product. In some implementations, the method further comprises transmitting, by the electronic device, operational data to said storage component. In some implementations, transmitting physiological data associated with said one or more physiological parameters along with said identification data to the external device comprises transmitting from the electronic device to the external device said physiological data associated with said one or more physiological parameters along with said identification data.

Disclosed herein is an electronic device configured to be secured to a subject and measure one or more physiological parameters of the subject. The electronic device can comprise a housing, one or more physiological sensors for measuring the one or more physiological parameters of the subject, and an antenna configured to allow the electronic device to wirelessly communicate with one or more separate devices. The housing can comprise: a bottom portion configured to face toward tissue of the subject when the electronic device is secured to the subject's body: a top portion opposite the bottom portion; a first side connected to the top portion along a first edge of the housing; a second side connected to the top portion along a second edge of the housing; a third side connected to the top portion along a third edge of the housing; a fourth side connected to the top portion along a fourth edge of the housing; and an interior surface extending along the top portion, bottom portion, first side, second side, third side, and fourth side. In some implementations, the antenna extends along the interior surface at least partially along each of the first edge, second edge, third edge, and fourth edge.

In the above electronic device or in other implementations as described herein, one or more of the following features can also be provided. In some implementations, the first side is opposite to the second side and the third side is opposite to the fourth side. In some implementations, the first side is generally parallel to the second side and the third side is generally parallel to the fourth side. In some implementations, the first side and the second side are each generally perpendicular to the third side and the fourth side. In some implementations, the antenna includes a first antenna leg and a second antenna leg separated from one another. In some implementations: the first antenna leg extends along said interior surface of the housing at least partially along the first edge, the third edge, and the fourth edge; and the second antenna leg extends along said interior surface of the housing at least partially along the second edge, the third edge, and the fourth edge. In some implementations: the first antenna leg includes a first end and a second end; the second antenna leg includes a first end and a second end; the first end of the first antenna leg is separated from the first end of the second antenna leg by a first gap; and the second end of the first antenna leg is separated from the second end of the second antenna leg by a second gap. In some implementations: the first ends of the first and second antenna legs are disposed along the fourth edge; and the second ends of the first and second antenna legs are disposed along the third edge. In some implementations, the first and second gaps are not aligned with one another. In some implementations, the first antenna leg includes a length and the second antenna leg includes a length that is substantially equal to the length of the first antenna leg. In some implementations, the first antenna leg extends along an entirety of the first edge and wherein the second antenna leg extends along an entirety of the second edge. In some implementations, substantially all of the antenna is disposed along portions of the interior surface of the housing along portions of the first, second, third, and fourth edges. In some implementations, the antenna includes a width that is between about 2.0 millimeters (mm) and about 10 mm. In some implementations, the antenna includes a width that is not greater than about 10 mm. In some implementations, the housing includes a first shell and a second shell, the first and second shells forming an interior of the housing, and wherein said first shell includes the top portion, the first edge, the second edge, the third edge, and the fourth edge. In some implementations, the first antenna leg includes a uniform width between the first and second ends of the first antenna leg, and wherein the second antenna leg includes a uniform width between the first and second ends of the second antenna leg. In some implementations, the antenna is a dipole antenna. In some implementations: a first corner of the housing is defined at an intersection of the first side and the fourth side; a second corner of the housing is defined at an intersection of the fourth side and the second side; a third corner of the housing is defined at an intersection of the second side and the third side; a fourth corner of the housing is defined at an intersection of the third side and the first side; the first antenna leg extends along the interior surface along the first and fourth corners; and the second antenna leg extends along the interior surface along the second and third corners. In some implementations, said one or more physiological sensors includes a pulse oximetry sensor. Disclosed herein is a wearable system including the electronic device including any of the features disclosed above or elsewhere herein and further including any of the bands disclosed herein.

Disclosed herein is an electronic device comprising a housing and a dipole antenna configured to allow the electronic device to wirelessly communicate with one or more separate devices. The housing can comprise: a bottom portion configured to face toward tissue of a subject when the electronic device is secured to a body of the subject; a top portion opposite the bottom portion; and an interior surface extending along the top portion. The dipole antenna can be arranged on (for example, painted on) the interior surface at least partially along the top portion.

In the above electronic device or in other implementations as described herein, one or more of the following features can also be provided. In some implementations, the dipole antenna is painted using a laser direct structuring (LDS) technique. In some implementations, the dipole antenna is painted using a laser enhanced plating (LEP) technique. In some implementations, the dipole antenna is painted with a metallic material, wherein the metallic material including at least one of gold and copper. In some implementations, the housing further comprises a first side connected to the top portion along a first edge of the housing, and wherein the dipole antenna is located along the interior surface along said first edge. In some implementations, the dipole antenna is painted along the interior surface and spaced from a circuit board of the electronic device. In some implementations, the dipole antenna is located on a corner of the interior surface of the top portion, wherein the dipole antenna extends along at least partially along a side of the interior surface.

Disclosed herein is an electronic device comprising a housing and an antenna configured to allow the electronic device to wirelessly communicate with one or more separate devices. The housing can comprise: a bottom portion configured to face toward tissue of a subject when the electronic device is secured to a body of the subject; a top portion opposite the bottom portion; and an interior surface extending along the top portion. The antenna can comprise a first arm and a second arm. The electronic device can further comprise: a first contact pad coupled to the first arm of the antenna; a second contact pad coupled to the second arm of the first arm of the antenna; a circuit board; a first electrical connector extending between and contacting the circuit board and the first contact pad; and a second electrical connector extending between and contacting the circuit board and the second contact pad.

In the above electronic device or in other implementations as described herein, one or more of the following features can also be provided. In some implementations, the first electrical connector is configured to apply a force to the first contact pad and allow transfer of an electrical signal to the first arm, and the second electrical connector is configured to apply a force to the second contact pad and allow transfer of the electrical signal to the second arm. In some implementations, the first contact pad is configured to distribute the force from the first electrical connector and the second contact pad is configured to distribute the force from the second electrical connector. In some implementations, the first contact pad is coupled to an end portion of the first arm. In some implementations, the first contact pad is soldered to the first arm and wherein the second contact pad is soldered to an end portion of the second arm. In some implementations, the first contact pad and the second contact pad each comprise one or more metallic materials.

Disclosed herein is an electronic device configured to be secured to a subject and measure one or more physiological parameters of the subject, the electronic device comprising: a housing including a top portion, a first side connected to the top portion, a second side connected to the top portion and opposite the first side, a third side connected to the top portion, a fourth side connected to the top portion and opposite the third side, and an interior surface extending along the first side, second side, third side, and fourth side; and an antenna configured to allow the electronic device to wirelessly communicate with one or more separate devices, the antenna extending along the interior surface at least partially along each of the first side, second side, third side, and fourth side. In some implementations, the top portion connects to the first, second, third, and fourth sides via edges, and the antenna extends at least partially along each of said edges.

Various combinations of the above and below recited features, embodiments, implementations, and aspects are also disclosed and contemplated by the present disclosure.

Additional implementations of the disclosure are described below in reference to the appended claims, which may serve as an additional summary of the disclosure.

In various implementations, systems and/or computer systems are disclosed that comprise a computer-readable storage medium having program instructions embodied therewith, and one or more processors configured to execute the program instructions to cause the systems and/or computer systems to perform operations comprising one or more aspects of the above- and/or below-described implementations (including one or more aspects of the appended claims).

In various implementations, methods and/or computer-implemented methods are disclosed in which, by one or more processors executing program instructions, one or more aspects of the above- and/or below-described implementations (including one or more aspects of the appended claims) are implemented and/or performed.

In various implementations, computer program products comprising a computer-readable storage medium are disclosed, wherein the computer-readable storage medium has program instructions embodied therewith, the program instructions executable by one or more processors to cause the one or more processors to perform operations comprising one or more aspects of the above- and/or below-described implementations (including one or more aspects of the appended claims).

### BRIEF DESCRIPTION OF THE DRAWINGS

Various implementations will be described hereinafter with reference to the accompanying drawings. These implementations are illustrated and described by example only, and are not intended to limit the scope of the disclosure. In the drawings, similar elements may have similar reference numerals.

FIGS. 1A-1B are schematic block diagrams illustrating implementations of a physiological monitoring system (PMS) in accordance with aspects of this disclosure.

FIG. 2 is a block diagram illustrating an implementation of a monitoring hub of a physiological monitoring system (PMS) in accordance with aspects of this disclosure.

FIG. 3 illustrates an example method for transferring physiological monitoring between a monitoring hub and a user device in accordance with aspects of this disclosure.

FIGS. 4 and 5A are flowcharts illustrating example methods associated with transferring physiological monitoring from an origin monitoring hub to a destination monitoring hub in accordance with aspects of this disclosure.

FIG. 5B is a flowchart illustrating an example method of monitoring a patient as the patient transitions between environments in accordance with aspects of this disclosure.

FIG. 5C is a flowchart illustrating an example method of accessing historical physiological data for monitoring a patient in accordance with aspects of this disclosure.

FIG. 6 illustrates a schematic diagram of certain features of an electronic device of a wearable system in accordance with aspects of this disclosure.

FIGS. 7A-7H illustrate a top perspective view, a bottom perspective view, a top view, a bottom view, a front view, a rear view, and side views, respectively, of an electronic device in accordance with aspects of this disclosure.

FIG. 7I-7J illustrate partially exploded views of the electronic device of FIGS. 7A-7H in accordance with aspects of this disclosure.

FIGS. 7K-7N illustrate portions of the electronic device of FIGS. 7A-7H in accordance with aspects of this disclosure.

FIG. 8 illustrates a schematic diagram of certain features of an electronic device of a wearable system in accordance with aspects of this disclosure.

FIGS. 9A-9B illustrate partially exploded views of an electronic device in accordance with aspects of this disclosure.

FIGS. 9C-9F illustrate portions of the electronic device of FIGS. 9A-9B in accordance with aspects of this disclosure.

FIGS. 9G-9H illustrate cross-section views of portions of the electronic device of FIGS. 9A-9B in accordance with aspects of this disclosure.

FIG. 10 illustrates a schematic diagram of certain features of a wearable system in accordance with aspects of this disclosure.

FIGS. 11A-11H illustrate a top perspective view, a bottom perspective view, a top view, a bottom view, side views, a front view, and a rear view, respectively, of a wearable system in accordance with aspects of this disclosure.

FIGS. 11I-11J illustrate perspective views of the wearable system of FIGS. 11A-11H with an electronic device removed from a wearable device in accordance with aspects of this disclosure.

FIGS. 12A-12H illustrate a top perspective view, a bottom perspective view, a top view, a bottom view, a front view, a rear view, and side views, respectively, of the electronic device of the wearable system of FIGS. 11I-11J in accordance with aspects of this disclosure.

FIG. 12I illustrates a partially exploded view of the electronic device of FIGS. 12A-12H in accordance with aspects of this disclosure.

FIGS. 12J-12K illustrate portions of the electronic device of FIGS. 12A-12H in accordance with aspects of this disclosure.

FIGS. 13A-13H illustrate top perspective views, bottom perspective views, a top view, a bottom view, and side views, respectively, of the wearable device of the wearable system of FIGS. 11I-11J in accordance with aspects of this disclosure.

FIG. 131 illustrates various implementations of a strap of the wearable device of FIGS. 13A-13B in accordance with aspects of this disclosure.

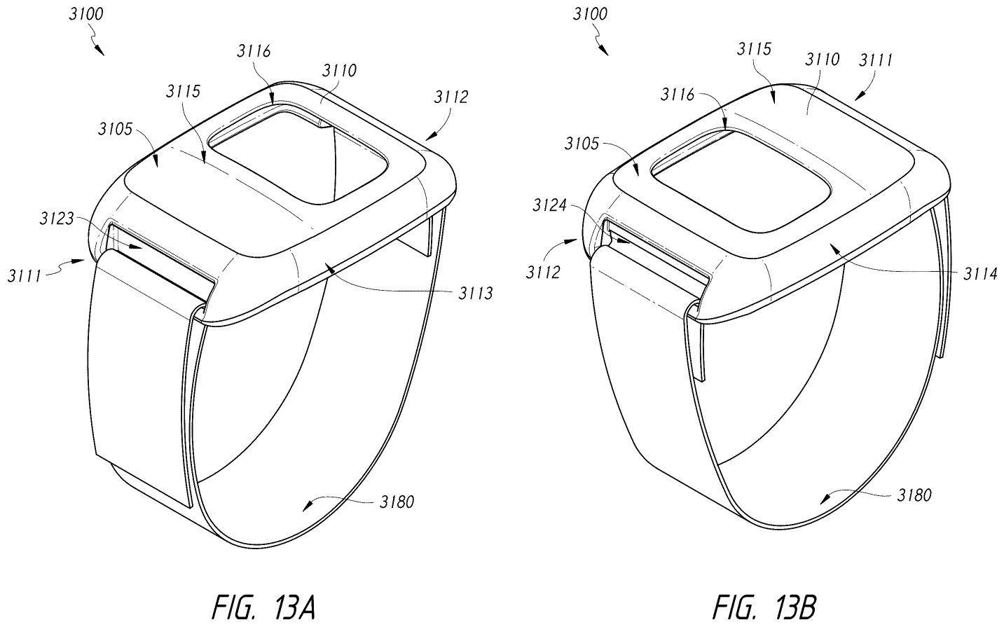

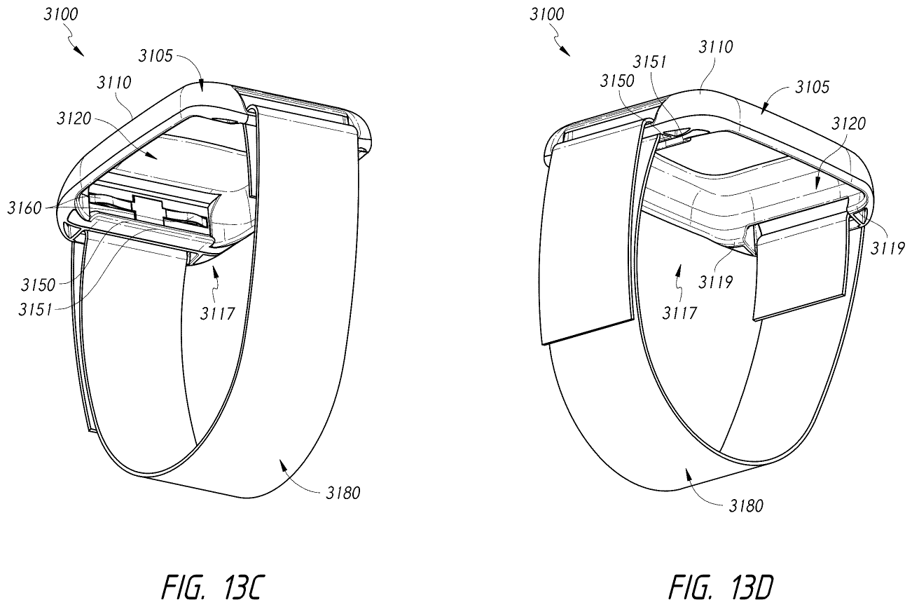

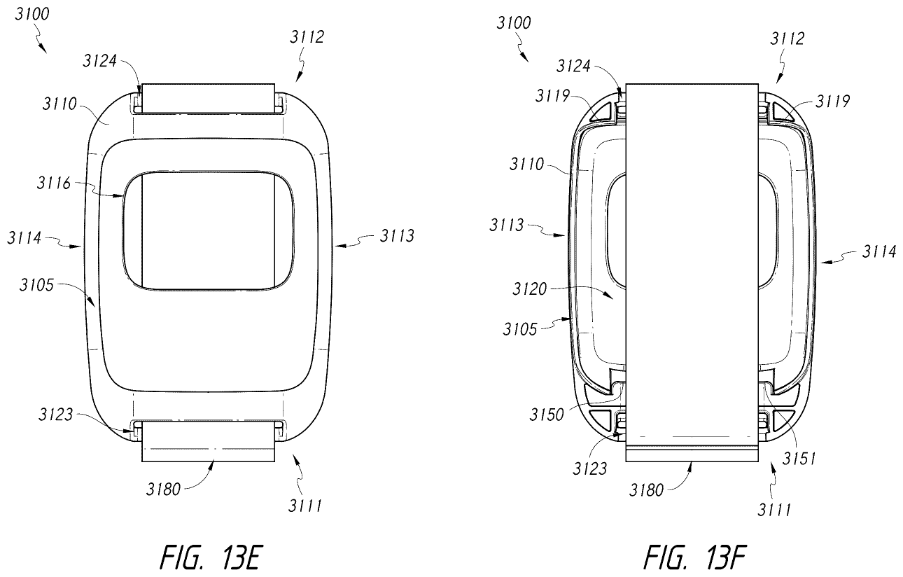

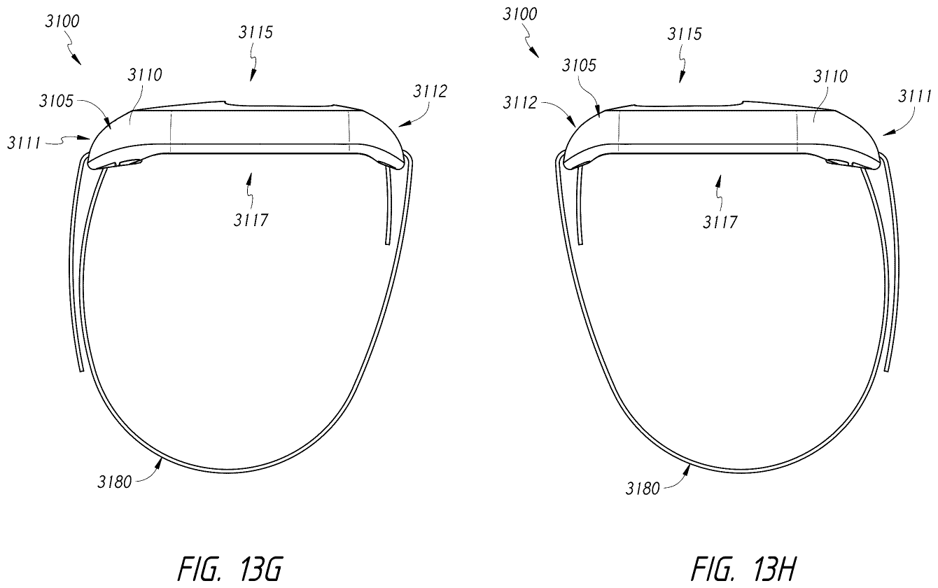

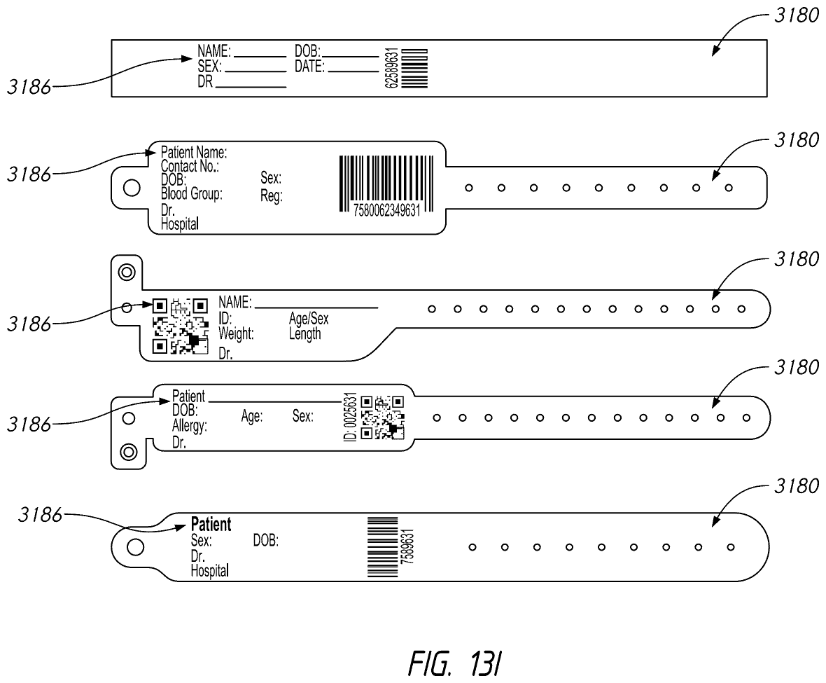

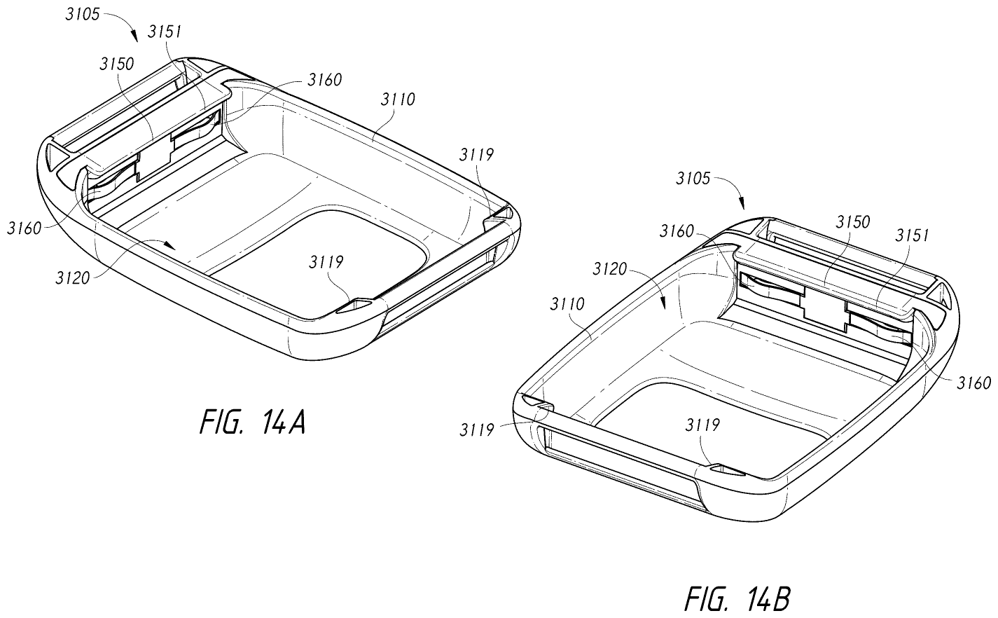

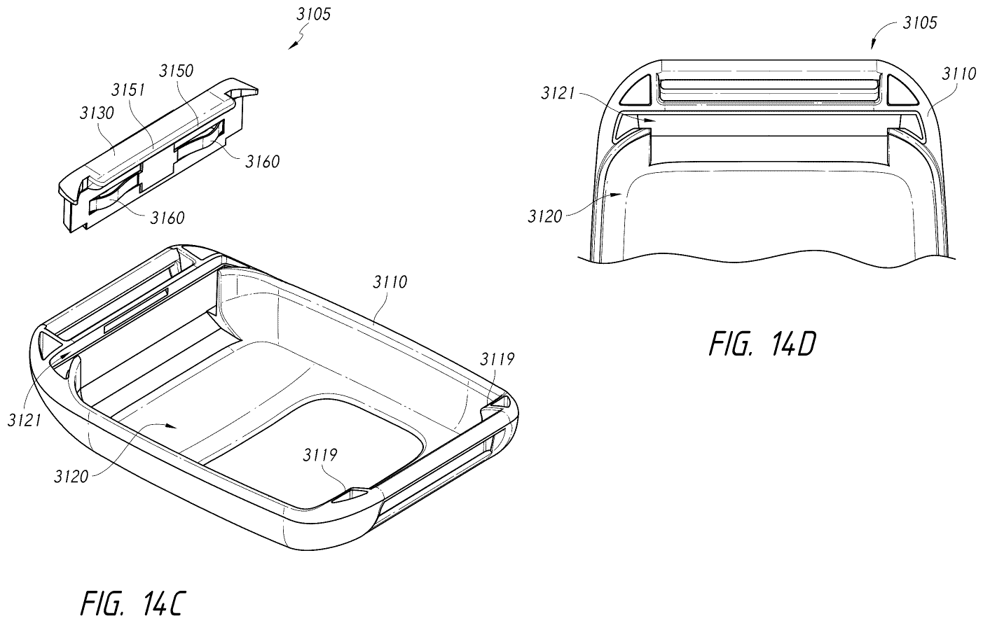

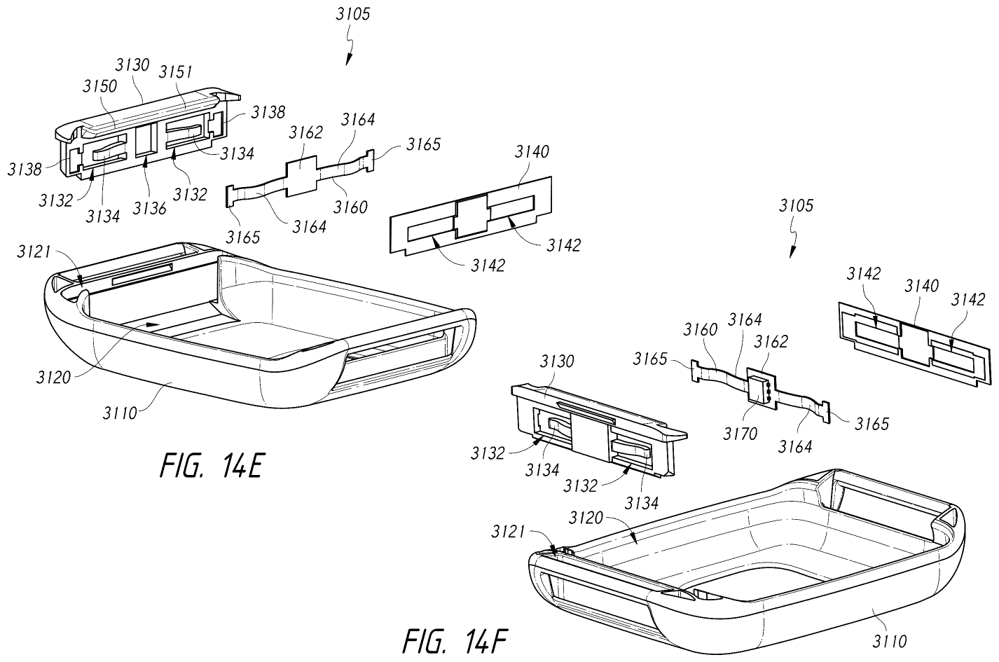

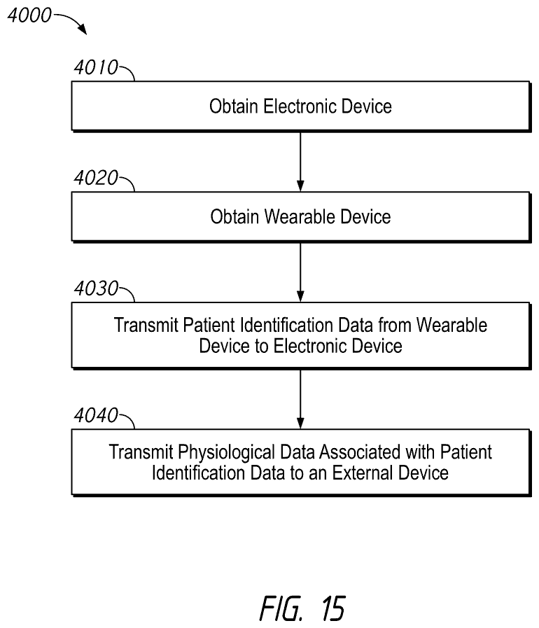

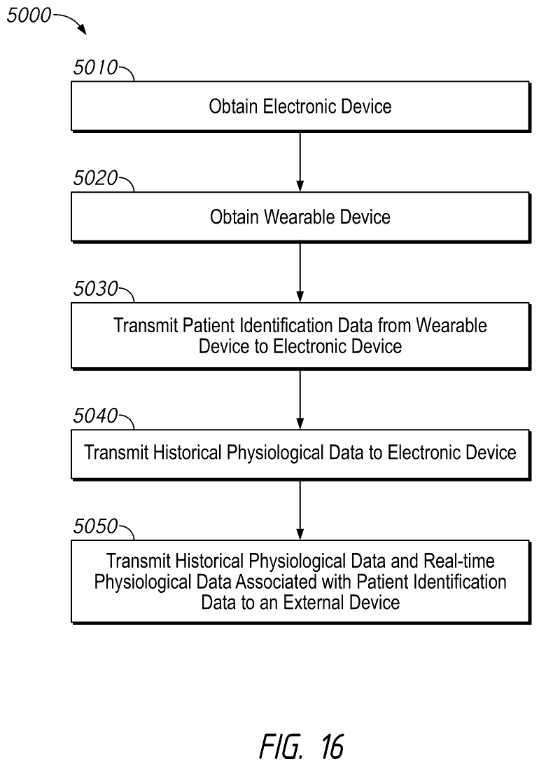

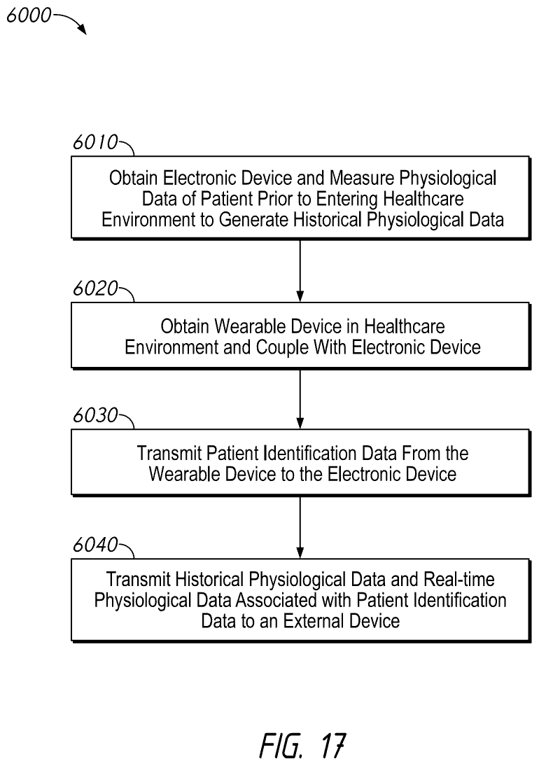

FIGS. 14A-14F illustrate various views of one or more portions of the wearable device of FIGS. 13A-13B in accordance with aspects of this disclosure.

FIGS. 15-17 are flowcharts illustrating example methods of data transmission in accordance with aspects of this disclosure.

### DETAILED DESCRIPTION

The present disclosure will now be described with reference to the accompanying figures, wherein like numerals may refer to like elements throughout. The following description is merely illustrative in nature and is in no way intended to limit the disclosure, its application, or uses. It should be understood that steps within a method may be executed in different order without altering the principles of the present disclosure. Furthermore, the devices, systems, and/or methods disclosed herein can include several novel features, no single one of which is solely responsible for its desirable attributes or which is essential to practicing the devices, systems, and/or methods disclosed herein. Additionally, the structures, systems, and/or devices described herein may be embodied as integrated components or as separate components.

A physiological monitoring system (PMS) can monitor a subject (which can also be referred to herein as a “patient” or a “wearer”) including physiological data of the subject. One or more physiological sensors can be coupled to the subject and can obtain physiological data of the subject. The one or more sensors can communicate physiological data to a monitoring hub which can display indicia of the physiological data. A user (which can also be referred to herein as a “provider,” a “caregiver,” a “healthcare provider,” a “nurse”, or a “doctor”) may desire to use another monitoring hub to monitor the subject such as to receive and display physiological data obtained from the sensors. The user can request the PMS to transfer physiological monitoring from the initial monitoring hub to the other monitoring hub. Described herein are systems, devices, methods, etc, for transferring physiological monitoring of a PMS from one monitoring hub to another monitoring hub and which can provide numerous benefits including improved physiological monitoring, improved health care services, and the like.

Advantageously, the systems, devices, and methods described herein can facilitate faster, simpler, and more efficient transfer of physiological monitoring from one monitoring hub to another monitoring hub. For example, a user may be able to transfer physiological monitoring from one monitoring hub to another monitoring hub without having to unplug, plug, and/or replug cables, wiring, etc. of the monitoring hubs and/or physiological sensors. As another example, a user may be able to transfer physiological monitoring from one monitoring hub to another monitoring hub without having to turn off and/or turn on devices connected to the monitoring hubs such as physiological sensors. As another example, a user may be able to transfer physiological monitoring from one monitoring hub to another monitoring hub without having to implement a time-consuming wireless pairing process. As another example, the PMS provides an intuitive and easy-to-use system for transferring physiological monitoring which can reduce time a health care provider must spend to oversee the subject's physiological monitoring and which can improve the quality of health care services provided to the subject.

Advantageously, the systems, devices, and methods described herein can reduce and/or eliminate data loss while transferring physiological monitoring from one monitoring hub to another monitoring hub. For example, a PMS may be configured to continuously monitor the physiology of the subject while transferring physiological monitoring from one monitoring hub to another monitoring hub. A PMS can be configured to retain (for example, store) physiological data obtained from sensor(s) before, during, and/or after transferring physiological monitoring between monitoring hubs. A PMS can be configured to exchange physiological data between monitoring hubs to allow a user to view historical physiological data in combination with present or real-time physiological data. For example, a monitoring hub of a PMS can be configured to display real-time physiological data received from sensors in addition to historical physiological data that was received from the sensor by another monitoring hub previous to transferring physiological monitoring as if the monitoring hub had been monitoring the subject the entire time and had received all physiological data from the sensors directly.

Advantageously, the systems, devices, and methods described herein for transferring physiological monitoring of a PMS can improve patient mobility. For example, a PMS may facilitate simple, quick, and efficient transfer of physiological monitoring from one monitoring hub (which may be at a fixed location such as a patient room in a hospital) to another monitoring hub (such as a portable monitoring hub) while continuing to monitor the patient which can allow the patient to relocate to a different location such as a different room in a hospital. Moreover, as a patient moves within an environment, such as a hospital, the PMS can facilitate automatic wireless communication between sensors coupled to the patient and monitoring hubs within a proximity of the patient. For example, sensors coupled to a patient may automatically establish a wireless connection, such as a Bluetooth connection, with the nearest monitoring hub as the patient moves around in an environment having multiple monitoring hubs. The monitoring hubs can communicate physiological data to a central server (as the monitoring hubs connect to the sensors) as the patient moves around in the environment. Accordingly, a central server may continuously receive physiological data for the patient as the patient moves around in an environment.

Advantageously, the systems, devices, and methods described herein can improve patient location tracking. For example, a PMS may monitor a patient's location, such as within a hospital, by identifying which monitoring hub(s) are wirelessly connected to sensors attached to the patient. As the patient moves within an environment the sensors coupled to the patient may wirelessly connect and/or communicate with various monitoring hubs, such as monitoring hubs within a close proximity to the sensors. Accordingly, a PMS can track a patient's location which can improve a quality of healthcare provided to the patient by quickly and efficiently having knowledge of the patient's location at all times, such as whether the patient is in a particular portion of a hospital they are supposed to be or have been assigned or scheduled to be, such as in an operating room.

Advantageously, the systems, devices, and methods described herein for transferring physiological monitoring of a PMS can facilitate removing, adding, replacing, and/or exchanging monitoring hubs within the PMS, for example when a monitoring hub is low on battery power and should be replaced by another monitoring hub to continue monitoring a subject, which can for example improve PMS performance as well as health care services.

Advantageously, the systems, devices, and methods described herein for transferring physiological monitoring of a PMS can improve quality control of health care services. For example, the PMS can be configured to verify the permissions associated with a user requesting to transfer physiological monitoring from one monitoring hub to another. For example, the PMS may be configured to reject a request to transfer physiological monitoring from a health care provider who does not have permission to relocate a subject being monitored and/or a health care provider who is not assigned to provide health care to the subject.

Advantageously, the systems, devices, methods described herein can improve fidelity of transferring physiological monitoring from one monitoring hub to another monitoring hub. For example, the PMS may be configured to verify a requesting user ID at a first monitoring hub and at a subsequent monitoring hub to ensure that the user IDs correspond (for example, match) which may ensure that the PMS transfers physiological monitoring to the correct monitoring hub (for example, to the monitoring with a requesting user ID that matches to the requesting user ID of the initial monitoring hub) which may advantageously improve transfer fidelity and accuracy in a system with numerous monitoring hubs and/or numerous requests to transfer occurring at or near the same time.

Also disclosed herein are wearable systems that can be used to measure, monitor, transmit (for example, wirelessly or via wired connection), process, and/or determine one or more physiological parameters of a subject. The disclosed wearable systems can generate one or more signals (which can also be referred to herein as “physiological data”) associated with and/or indicative of one or more physiological parameters of a subject and process such one or more signals to determine such physiological parameters. In some implementations, the disclosed wearable systems can generate and transmit (for example, wirelessly or via wired connection) one or more signals associated with and/or indicative of one or more physiological parameters of a subject to a separate or external monitoring, computing, and/or electronic device (for example, a mobile phone or a monitoring hub) which is capable of processing and/or determining such physiological parameters based on the transmitted signals. Any of the disclosed wearable systems and/or devices in communication with the wearable systems can include hardware and/or software capable of determining and/or monitoring a variety of physiological parameters, including but not limited to blood oxygenation levels in veins and/or arteries, heart rate, blood flow, respiratory rates, an electrocardiogram (ECG) and/or other physiological parameters or characteristics such as those discussed herein. Any of the wearable systems described herein can include and/or employ pulse oximetry (for example, via an optical sensor) to measure physiological parameters of the subject and/or to generate, transmit, and/or process one or more signals associated with and/or indicative of such physiological parameters and/or to determine such physiological parameters. As discussed herein, such optical sensor can include one or more emitters configured to emit optical radiation (for example, light) of one or more wavelengths (for example, wavelength(s) in the visible spectrum, near infrared wavelength(s), infrared wavelength(s), far infrared wavelength(s), etc.) and one or more detectors configured to detect at least a portion of the emitted optical radiation after attenuation, reflecting off of, and/or passing through tissue of the subject.

Advantageously, the systems and/or devices thereof described herein can associate patient identification data with physiological data generated by the system and/or physiological parameters measured by the system. Such association can advantageously occur automatically. For example, such association can occur when an electronic device (for example, any of those described herein) is secured to a wearable device as described in more detail herein. Further to this example, such association can occur as a result of contact between electrical contacts of the electronic device and electrical contacts of the wearable device, which can facilitate transfer of the patient identification data from a storage component of the wearable device to the electronic device. As another example, such association can occur when an electronic device as described herein is within a proximity of a wearable device as described herein. Further to this example, such association can occur as a result of wireless transfer of patient identification data stored by a storage component of the wearable device to an electronic device, which can be facilitated by communication components of each of the electronic device and wearable device.

Advantageously, the systems and/or devices thereof described herein can associate patient identification data with historical physiological data. Such historical physiological data can be data generated prior to the patient entering a healthcare environment and/or data generated during a prior visit to the healthcare environment. Furthermore, such historical data can be generated by a system and/or device that is different than the system and/or device used to generate physiological data in the healthcare environment, or it can be generated by a system and/or device that is the same or part of the system used to generate physiological data in the healthcare environment. For example, the same electronic device used to generate historical physiological data of the patient in an at-home environment can be used to generate physiological data of the patient in the healthcare environment. In such example, the wearable device used to secure the electronic device to the patient can be different and/or changed between the at-home and the healthcare environment.

Advantageously, the systems and/or devices thereof described herein can associate patient identification data with historical physiological parameters. Such historical physiological parameters can be parameters measured prior to the patient entering a healthcare environment and/or parameters measured during a prior visit to the healthcare environment. Furthermore, such historical parameters can be measured by a system and/or device that is different than the system and/or device used to measure physiological parameters in the healthcare environment, or it can be measured by a system and/or device that is the same or part of the system used to measure physiological parameters in the healthcare environment. For example, the same electronic device used to measure historical physiological parameters of the patient in an at-home environment can be used to measure physiological parameters of the patient in the healthcare environment. In such example, the wearable device used to secure the electronic device to the patient can be different and/or changed between the at-home and the healthcare environment. Advantageously, the systems and/or devices thereof described herein can transfer physiological data and/or physiological parameters of a patient to an external device with patient identification data of the patient. Such physiological data and/or physiological parameters can be historical and/or real-time. Such transfer of physiological data and/or physiological parameters can be performed at the same time or sequentially with the transfer of patient identification data. The systems and/or devices thereof described herein can associate the physiological data and/or physiological parameters of the patient with patient identification data of the patient. In some implementations, the external device associates the physiological data and/or physiological parameters of the patient with patient identification data of the patient after receiving such information from the systems and/or devices thereof described herein.

To facilitate an understanding of the systems and methods discussed herein, several terms are described below. These terms, as well as other terms used herein, should be construed to include the provided descriptions, the ordinary and customary meanings of the terms, and/or any other implied meaning for the respective terms, wherein such construction is consistent with context of the term. Thus, the descriptions below do not limit the meaning of these terms, but only provide example descriptions.

In some implementations, a sensor can be embodied in or comprise an electronic device, a portion of an electronic device, a wearable device, a portion of a wearable device, a wearable system, or a portion of a wearable system. A sensor can be implemented as or comprise any of the wearable systems or portions thereof (such as an electronic device of a wearable system) described herein. A sensor can be implemented as or comprise a wireless wearable device or a wireless wearable system. A wearable device and/or an electronic device can include one or more sensors. A wearable device and/or an electronic device can comprise a wearable hub in communication with one or more sensors. A sensor can be embodied as or comprise an auricular device such as an earbud, earpiece, headphone, earphone, or the like. A sensor can be embodied as or comprise a wrist-worn device such as a smartwatch. A sensor can comprise a physiological sensor. A sensor can include one or more physiological sensors configured to generate physiological data of physiological parameters (for example, generate physiological data to measure one or more physiological parameters). A sensor can include acoustic sensors, optical sensors, inertial sensors, temperatures sensors, electrical sensors, voltage sensors, impedance sensors, etc. A sensor can include an oximeter. A sensor can include a photoplethysmography (PPG) sensor configured to measure volumetric variations in blood circulation and derive one or more parameters therefrom, such as pulse rate, blood pressure, respiration rate, cardiac output, perfusion index, pleth variability index, PPG waveform data, blood oxygen saturation, etc. A sensor can include one or more optical emitters configured to emit optical radiation of a plurality of wavelengths, which can include visible light. A sensor can include one or more optical detectors configured to detect optical radiation attenuated by the tissue of subject (which may have been emitted by optical emitters) and generate data relating to the pulsatile characteristics of the subject, including blood oxygen saturation, hydration, hemoglobin content, etc. A sensor can include electrocardiogram (ECG) sensors, including one or more electrodes, configured to measure electrical activity of the subject, such as cardiac signals. A sensor can include electroencephalography (EEG) sensors. A sensor can measure and/or generate data relating to respiration rate, blood oxygen saturation (for example, SpO2), heart rate, pulse rate, skin temperature, core body temperature, spatial orientation, or the like. A sensor can be coupled to a subject. A sensor may be attached to a subject. A sensor can be donned by a subject. A sensor can be worn by a subject. A sensor can be secured to a subject by adhesion. A sensor can be secured to a subject by one or more straps. A sensor may be worn on and/or attached to a finger, wrist, arm, forearm, head, forehead, ear, chest, back, torso, stomach, leg, ankle, foot, toe, or other body portion of a subject. A plurality of sensors can be disposed within a same housing or device. Sensors may be disposed within separate housings or devices.

In some implementations, a monitoring hub can comprise an electronic device configured to facilitate physiological monitoring of a subject. A monitoring hub can display indicia corresponding to physiological data of the subject. A monitoring hub can be mobile. A monitoring hub can be portable. A monitoring hub may comprise a hand-held device. A monitoring hub may be carried by a user. A monitoring hub can be mounted to a wall. A monitoring hub may be an in-room display. A monitoring hub may be stationary. A monitoring hub may be at a fixed location. A monitoring hub may comprise a display, tablet, monitor, PC, phone, laptop, wearable device, such as a smartwatch, user device, or the like. A monitoring hub may communicate with one or more remote computing devices via one or more wireless communication protocols. A monitoring hub may communicate with a remote server. A monitoring hub may communicate with one or more sensors. A monitoring hub may also be referred to herein as a hub, an electronic device, a display device, a display terminal, a monitoring device, or the like. An environment, such as a healthcare facility may have a plurality of monitoring hubs distributed throughout the environment at various locations such as mounted to walls, mounted to doors, distributed in rooms, distributed along hallways, carried by personnel, coupled to beds, coupled to subjects, or the like.

In some implementations, an origin monitoring hub can refer to a monitoring hub in wireless communication with one or more sensors prior to transferring physiological monitoring to another monitoring hub. An origin monitoring hub can comprise any of the example monitoring hubs shown and/or described herein including structural and/or operational features of any of the example monitoring hubs shown and/or described herein. An origin monitoring hub may also be referred to herein as a first monitoring hub, an initial monitoring hub, or the like.

In some implementations, a destination monitoring hub can refer to a monitoring hub in wireless communication with one or more sensors subsequent to transferring physiological monitoring from another monitoring hub. A destination monitoring hub can comprise any of the example monitoring hubs shown and/or described herein including structural and/or operational features of any of the example monitoring hubs shown and/or described herein. A destination monitoring hub may also be referred to herein as a second monitoring hub, a subsequent monitoring hub, another monitoring hub, or the like.

A destination monitoring hub may comprise a different type of monitoring hub than an origin monitoring hub. For example, one of the destination monitoring hub or the origin monitoring hub may comprise a mobile monitoring hub while the other of the destination monitoring hub or the origin monitoring hub comprises a monitoring hub in a fixed location, such as a wall-mounted monitoring hub or an in-room monitoring hub. A destination monitoring hub may comprise a same type of monitoring hub as an origin monitoring hub. For example, a destination monitoring hub and an origin monitoring hub may both comprise mobile monitoring hubs.

In some implementations, “transferring physiological monitoring” can refer to transferring the monitoring, displaying, and/or collection of physiological data from an origin monitoring hub to a destination monitoring hub. Transferring physiological monitoring can include transferring a wireless connection between sensor(s) and an origin monitoring hub to sensor(s) and a destination monitoring hub. Transferring physiological monitoring can include updating or changing a wireless connection of physiological sensor(s) and/or updating or changing a wireless connection of monitoring hub(s). Transferring physiological monitoring can include terminating wireless communication between an origin monitoring hub and sensors. Transferring physiological monitoring can include establishing a wireless communication between a destination monitoring hub and sensors. In some implementations, transferring physiological monitoring can include transferring less than all of the wireless connections to sensors from an origin monitoring hub to a destination monitoring hub. In some implementations, transferring physiological monitoring can include transferring all of the wireless connections to sensors from an origin monitoring hub to a destination monitoring hub.

In some implementations, a transfer request can include a request to transfer physiological monitoring from an origin monitoring hub to a destination monitoring hub. A transfer request can be received via a user input at a monitoring hub. For example, a user may press a button on one or more monitoring hubs to initiate a transfer request. In some implementations, a transfer request may comprise a non-contact or minimal-contact user input, such as a wireless communication signal, facial recognition, eye recognition, fingerprint recognition, gesture recognition, voice recognition, or the like. A transfer request can be received at a location and/or computing device that is remote to a monitoring hub. A transfer request may initiate transferring physiological monitoring.

In some implementations, identification data can include data generated and/or received via a monitoring hub when transferring physiological monitoring. Identification data can include data that is associated with a user and can be used to identify the user. Such identification data can be referred to herein as “user identification data”. Identification data can comprise a user ID. Identification data can include a tag, marker, serial number, bar code, QR code, facial recognition, fingerprint recognition, voice recognition, eye recognition, gesture recognition, or the like. Identification data may be unique to a user. Identification data may be unique to a group of users (and may be the same for individuals within the group). Identification data may be used to identify a group to which the user belongs. Identification data may comprise or indicate permissions associated with a user such as permission or authority to transfer physiological monitoring. Identification data can include a reason (for example, provided by a requesting user) for requesting a transfer. A computing device, such as a monitoring hub, can receive identification data via one or more wireless communication protocols such as near field communication (NFC) or radio frequency identification (RFID). For example, a user may place a badge configured for wireless communication in proximity to a monitoring hub to be detected by the monitoring hub. A computing device, such as a monitoring hub, can receive identification data via manual user input at a monitoring hub. For example, a user may enter their identification data at the monitoring hub via a keyboard, user interface, touchscreen, or the like. A computing device, such as a monitoring hub, can receive identification data via one or more biological markers. For example, a user may scan their finger, eye, face, or speak as their identification data to be identified at the monitoring hub. Identification data can be linked or paired with a transfer request.

Identification data can include data that is associated with a subject and can be used to identify the subject. Such identification data may be also referred to as “subject identification data” or “patient identification data”. Such identification data can include one or more of a name, assigned identification number or health record number, date of birth, phone number, social security number, address, photo, dates of hospitalizations or visits to a healthcare environment, name of attending physicians or care providers, demographics, diagnoses, problems list, progress notes, medications, vital signs, laboratory data, tests, allergies, immunizations, treatment plans, tag, marker, serial number, bar code, QR code, facial recognition, fingerprint recognition, voice recognition, eye recognition, gesture recognition, biometric data, or the like. Identification data associated with the subject can include data from the subject's electronic health record. Identification data associated with the subject may be unique to the subject.

In some implementations, a transfer request status may indicate a status of a request to transfer physiological monitoring. A transfer request status can include an approved or not approved status. In some implementations, a transfer request may be approved if identification data from a first monitoring hub matches identification data from a second monitoring hub. A transfer request may be approved if a requesting user has appropriate permissions to perform the transfer. In some implementations, a transfer request may not be approved if identification data from a first monitoring hub does not match identification data from a second monitoring hub. A transfer request may not be approved if a requesting user does not have appropriate permissions to perform the transfer.

In some implementations, wireless communication configuration data can comprise data used to establish wireless communication between one or more computing devices. For example, a monitoring hub and sensor may implement wireless communication configuration data to communicate with each other via one or more wireless communication protocols. Wireless communication configuration data can include device addresses of one or more computing devices such as monitoring hubs and/or sensors. Wireless communication configuration data can include access codes such as one or more of Inquiry Access Codes (IAC), Device Access Codes (DAC), and Channel Access Codes (CAC). An access code can include and/or be derived from a device address. Wireless communication configuration data can include link keys. Wireless communication configuration data can include clock data such as frequencies at which computing devices will communicate (for example, to transmit data). Wireless communication configuration data may also be referred to herein as wireless communication data or communication data or wireless configuration data or configuration data.

In some implementations, a device address can facilitate wireless communication between computing devices. A device address may be associated with a computing device. A device address may be unique to a computing device. A device address can comprise an IP address. A device address can comprise a MAC address. A device address can comprise a serial ID associated with a computing device. A device address can comprise a Bluetooth Address (BD_ADDR). A device address can comprise an LAP value. A device address, or derivation thereof, may form at least a portion of an access code.

In some implementations, a link key may facilitate wireless communication between computing devices. A link key can authenticate one or more computing devices with each other. A link key can encrypt data exchanged wirelessly between one or more computing devices. A link key can comprise a Long-Term Key (LTK).

In some implementations, physiological data can include data generated by one or more sensors. In some implementations, physiological data can include data generated by one or more wearable devices, one or more wearable systems, or a portion thereof. Physiological data can correspond to a subject. Physiological data can include raw data, partially processed data, and/or fully processed data. Physiological data can include physiological parameters. Physiological data can include data relating to heart rate, respiration rate, blood pressure, blood oxygen saturation, hemoglobin content, PPG data, ECG data, EEG data, temperature, subject orientation, subject position, subject movement, depth-of-consciousness, capnography data, acoustic data, motion data, as non-limiting examples. Physiological data can include historical physiological data. Historical physiological data can include physiological data generated by a sensor over a time frame preceding a present time. Historical physiological data can include data corresponding to a time frame of months, days, less than 24 hours, less than 12 hours, less than 1 hour, less than 30 minutes, less than 10 minutes, less than 5 minutes, less than 2 minutes, less than 1 minute, less than 30 seconds, less than 15 seconds, less than 10 seconds, less than 5 seconds, or less than 1 second. Physiological data can include real-time physiological data. Real-time physiological data can include physiological data transmitted and/or received at a substantially similar time as the physiological data is generated by a sensor, for example, such that any difference in time may be imperceptible to human senses.

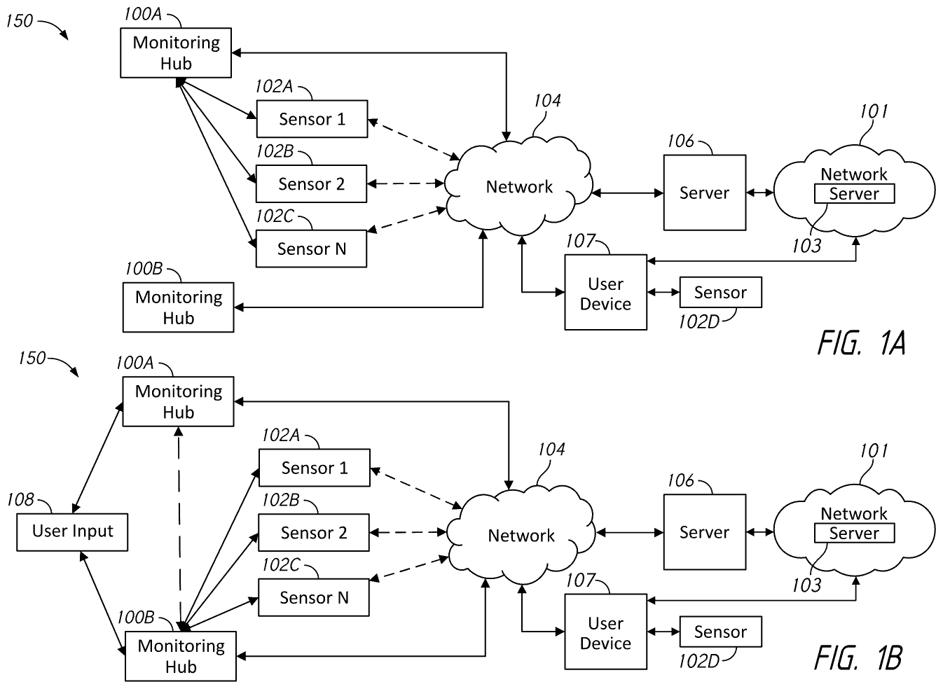

FIG. 1A is a schematic block diagram illustrating an example implementation of a physiological monitoring system (PMS) 150. The PMS 150 can include a monitoring hub 100A, a monitoring hub 100B, one or more sensors 102 (for example, sensors 102A, 102B, 102C), a network 104, a user device 107, and one or more servers 106. In some implementations, the PMS 150 may include only two monitoring hubs (for example, hubs 100A, 100B). In some implementations, the PMS 150 may include more than two monitoring hubs. In some implementations, the PMS 150 may include only one monitoring hub. The PMS 150 can include and/or be in communication with a separate network 101 and server 103. For example, the server 106 of PMS 150 can communicate with server 103 to join network 104 and network 101. The network 104 can be associated with a healthcare environment such as a hospital and may be referred to as a healthcare environment network. The network 101 can be associated with a home environment and can be referred to as a home environment network.

The monitoring hub 100A can communicate with the one or more sensors 102. In some implementations the monitoring hub 100A may communicate with the one or more sensors 102 via a wireless communication protocol such as WiFi, Bluetooth, near field communication (NFC), radio frequency identification (RFID), cellular, 1G, 2G, 3G, 4G, 5G, and/or Zigbee. In some implementations, the sensor(s) 102 may be a “slave” in a master-slave communication relationship such as a Bluetooth communication protocol with the monitoring hub 100A. In some implementations, the sensor(s) 102 may communicate with only one device at a time (for example, a “master” device) such as a monitoring hub. The monitoring hub 100A may communicate data to the one or more sensors 102 and/or receive data from the one or more sensors 102. For example, the monitoring hub 100A may receive physiological data from the one or more sensors 102. As another example, the monitoring hub 100A may receive communication data (for example, device addresses of the one or more sensors 102) from the one or more sensors 102 and/or communicate communication data (for example, device addresses of the monitoring hubs 100A, 100B) to the one or more sensors 102. In the example implementation illustrated in FIG. 1A, monitoring hub 100B has not established direct wireless communication with the one or more sensors 102.

The monitoring hubs 100A, 100B can communicate with the server 106 via a network 104. The network 104 can include any one or more communications networks. The network 104 can include a plurality of computing devices configured to communicate with one another. The network 104 can include routers. The network 104 can include the Internet. The network 104 can include a cellular network. The network 104 can include any combination of a body area network (for example, implementing human body communication with capacitive coupling via the tissue of a user's body), a local area network (“LAN”) and/or a wide area network (“WAN”), or the like. Accordingly, various components of the PMS 150 can communicate with one another directly or indirectly via any appropriate communications links and/or networks, such as network 104 (for example, one or more communications links, one or more computer networks, one or more wired or wireless connections, the Internet, any combination of the foregoing, and/or the like).

Communication over the network 104 can include a variety of communication protocols, including wired communication, wireless communication, wire-like communication, near-field communication (such as inductive coupling between coils of wire or capacitive coupling between conductive electrodes), and far-field communication (such as transferring energy via electromagnetic radiation (for example, radio waves)). Example communication protocols can include Wi-Fi, Bluetooth®, ZigBee®, Z-wave®, cellular telephony, such as long-term evolution (LTE) and/or 1G, 2G, 3G, 4G, 5G, etc., infrared, radio frequency identification (RFID), satellite transmission, inductive coupling, capacitive coupling, proprietary protocols, combinations of the same, and the like.

The monitoring hubs 100A, 100B can, via the network 104, communicate data to the server 106 and/or receive data from the server 106 including communication data (for example, device addresses and/or link keys corresponding to the sensors 102 and/or monitoring hubs 100), physiological data, identification data (user ID), transfer requests, request approval status, or the like. In some implementations, a monitoring hub 100 may communicate with the server 106 via a different wireless communication protocol than which it communicates with the one or more sensors 102. For example, a monitoring hub 100 may communicate with the server 106 via a first wireless communication protocol, such as WiFi, and may communicate with the one or more sensors 102 via a second wireless communication protocol, such as Bluetooth.

In some implementations, the sensors 102 may optionally communicate with the server 106 via the network 104. For example, the sensors 102 may communicate physiological data to the server 106 and/or receive communication data (for example, device address of a monitoring hub) from the server 106. In some implementations, the sensors 102 may not communicate directly with the server 106. In some implementations, data may be transmitted from the sensor(s) 102 to the server 106 via a monitoring hub 100, or vice versa.

In some implementations, the monitoring hub 100A may be portable or mobile. For example, the monitoring hub 100A may be sized, shaped, and/or include a housing or casing to facilitate carrying the monitoring hub 100A such as by hand. In some implementations, the monitoring hub 100A may be stationary or fixed in a location. For example, the monitoring hub 100A may be mounted to a wall. In some implementations, the monitoring hub 100B may include similar structural and/or operational features as monitoring hub 100A. The monitoring hub 100A may be referred to as an origin monitoring hub. The monitoring hub 100B may be referred to herein as a destination monitoring hub.

The one or more sensors 102 can include various types of sensors configured to collect physiological data of a subject. The one or more sensors 102 can attach or couple to different parts of a subject such as, but not limited to, arms, legs, torso, chest, head, neck, fingers, forehead, and the like. The one or more sensors 102 can collect patient physiological data including, but not limited to, data relating to heart rate, pulse rate, respiration rate, blood pressure, blood oxygen saturation, hemoglobin content, ECG data, EEG data, temperature, subject orientation, subject position, subject movement, as non-limiting examples, and the like. The one or more sensors 102 can transmit physiological data to the monitoring hub 100A, monitoring hub 100B, and/or to the server 106 in real-time as the one or more sensors 102 collect the data. In some implementations, one or more sensors 102 can include processors that can fully or partially process the data obtained by the sensors 102. Any of the wearable systems described herein (such as wearable system 3000) and/or portions thereof (such as electronic device 3200) can be implementations of sensors 102.

The server 106 may comprise one or more computing devices including one or more hardware processors (which may also be referred to as “hardware computer processors” herein). The server 106 may comprise program instructions configured to cause the server 106 to perform one or more operations when executed by the hardware processors. The server 106 may include, and/or have access to (for example, be in communication with) a database or storage component or storage system which can include any computer readable storage medium and/or device (or collection of data storage mediums and/or devices), including, but not limited to, one or more memory devices that store data, including without limitation, dynamic and/or static random access memory (RAM), programmable read-only memory (PROM), erasable programmable read-only memory (EPROM), electrically erasable programmable read-only memory (EEPROM), optical disks (for example, CD-ROM, DVD-ROM, etc.), magnetic disks (for example, hard disks, floppy disks, etc.), memory circuits (for example, solid state drives, random-access memory (RAM), etc.), and/or the like. In some implementations, the server 106 may include and/or be in communication with a hosted storage environment that includes a collection of physical data storage devices that may be remotely accessible and may be rapidly provisioned as needed (commonly referred to as “cloud” storage). Data stored in and/or accessible by the server 106 can include physiological data including historical physiological data previously obtained by the one or more sensors 102 and/or communication data including, for example, link keys and/or device addresses associated with monitoring hubs, sensors, or the like.

In some implementations, the network 104 may comprise and/or be in communication with an electronic medical records (EMR). In some implementations, the server 106 may comprise and/or be in communication with an EMR. In some implementations, the one or more of the monitoring hubs 100A, 100B may be in communication with an EMR. An EMR can comprise a propriety EMR. An EMR can comprise an EMR associated with a hospital. An EMR can store data including medical records.

In some implementations, the server 106 can manage and/or process data received from monitoring hub 100A and/or 100B. For example, the server 106 can process physiological data originating at sensors 102 and received from monitoring hub 100A and/or 100B to generate processed physiological data which can include physiological parameter values, alarms, alerts, notifications, trends, comparisons, or the like. In some implementations, the server 106 can generate user interface data corresponding to physiological data for rendering user interfaces comprising indicia of the physiological data. Advantageously, the server 106 may act as a central processing computing system that processes data from distributed computing devices (for example, monitoring hubs 110, sensors 102, user device 107, etc.) which may reduce local processing requirements on the distributed computing devices. In some implementations, the server 106 may store data received from monitoring hub 110A and/or 110B, such as physiological data and/or wireless configuration data. Advantageously, the server 106 may act as a central storage (for example, database) such that distributed computing devices (for example, monitoring hubs 110, sensors 102, user device 107, etc.) can access the same data stored at the server 106 which may reduce local storage requirements at the distributed computing devices.

The network 101 can include any one or more communications networks. The network 101 can include a plurality of computing devices configured to communicate with one another. The network 101 can include routers. The network 101 can include the Internet. The network 101 can include a cellular network. The network 101 can include any combination of a body area network (for example, implementing human body communication with capacitive coupling via the tissue of a user's body), a local area network (“LAN”) and/or a wide area network (“WAN”), or the like. Accordingly, various devices can communicate with one another directly or indirectly via any appropriate communications links and/or networks, such as network 101 (for example, one or more communications links, one or more computer networks, one or more wired or wireless connections, the Internet, any combination of the foregoing, and/or the like). Communication over the network 101 can include a variety of communication protocols, including wired communication, wireless communication, wire-like communication, near-field communication (such as inductive coupling between coils of wire or capacitive coupling between conductive electrodes), and far-field communication (such as transferring energy via electromagnetic radiation (for example, radio waves)). Example communication protocols can include Wi-Fi, Bluetooth®, ZigBee®, Z-wave®, cellular telephony, such as long-term evolution (LTE) and/or 1G, 2G, 3G, 4G, 5G, etc., infrared, radio frequency identification (RFID), satellite transmission, inductive coupling, capacitive coupling, proprietary protocols, combinations of the same, and the like.

The server 103 may comprise one or more computing devices including one or more hardware processors. The server 103 may comprise program instructions configured to cause the server 103 to perform one or more operations when executed by the hardware processors. The server 103 may include, and/or have access to (for example, be in communication with) a database or storage component or storage system which can include any computer readable storage medium and/or device (or collection of data storage mediums and/or devices), including, but not limited to, one or more memory devices that store data, including without limitation, dynamic and/or static random access memory (RAM), programmable read-only memory (PROM), erasable programmable read-only memory (EPROM), electrically erasable programmable read-only memory (EEPROM), optical disks (for example, CD-ROM, DVD-ROM, etc.), magnetic disks (for example, hard disks, floppy disks, etc.), memory circuits (for example, solid state drives, random-access memory (RAM), etc.), and/or the like. In some implementations, the server 103 may include and/or be in communication with a hosted storage environment that includes a collection of physical data storage devices that may be remotely accessible and may be rapidly provisioned as needed (commonly referred to as “cloud” storage). Data stored in and/or accessible by the server 103 can include physiological data including historical physiological data.

The user device 107 may be a phone, laptop, PC, wearable device, smartwatch, tablet, or the like. The user device 107 can include a display. The user device 107 can include one more hardware processors configured to execute program instructions to cause the user device 107 to perform operations. In some implementations, the user device 107 can comprise one or more sensors integrated within a same structural device or housing. For example, the user device 107 can be a smartwatch having one or more sensors (for example, pulse oximeters) integrated within a housing of the smartwatch. In some implementations, the user device 107 can process data originating from sensors to generate processed physiological data and/or physiological parameters. The user device 107 can communicate with sensor 102D and can receive processed and/or unprocessed physiological data from the sensor 102D. The sensor 102D can be a wearable device that can be coupled to a user via adhesive, straps, clips, etc. The sensor 102D can be a finger sensor. The sensor 102D can be integrated with a non-wearable device, such as an audio speaker. The user device 107 can communicate data originating from sensor 102D to the server 103 over network 101. In some implementations, the sensor 102 can directly communicate with server 103 over network 101.

The server 103 can collect physiological data from the user device 107 and/or sensor 102D such as before and/or after a user is being monitored in a healthcare facility. The network 101 and/or server 103 may be associated with a user's home. For example, the network 101 can be a private network owned and/or managed by a user and the server 103 can collect data from the user device 107 and/or sensor 102D when the user is at their home connected to their network 101. The network 101 and/or server 103 may not be associated with a user's home. For example, the network 101 can be a public network and the server 103 (which can be remote to the user's home) can collect data from the user device 107 and/or sensor 102D when the user is connected to public network 101 whether the user is at their home or in another location from their home. The network 101 (with server 103) can be referred to as a home network 101 whether associated with the user's home or not.

In some implementations, the server 103 may only connect to approved devices over network 101. In some implementations, the server 103 can connect to approved or unapproved devices but may only receive data from approved devices. In some implementations, the server 103 can received data from approved or unapproved devices but may only store data from approved devices as persistent data in long term storage. Approved devices can include devices that are medically approved by an organization with regulatory oversight such as the U.S. Food and Drug Administration (FDA). The server 103 can indicate whether data originated from an approved or unapproved device, such as by marking the data with metadata. Data originating from an approved device may be more accurate, reliable, and medically safe than data originating from an unapproved device. Advantageously, the server 103 can manage and/or process data appropriately depending on whether the data is from an approved device and in some implementations can reject data from an unapproved device.

After generating and storing physiological data at the server 103 (such as at home or elsewhere), a user can be admitted to a healthcare facility to be monitored by PMS 150. When the user is admitted to the healthcare facility a request can be generated to the server 106 to implement an admission routine. The request can be generated with the user device 107 such as when the user device 107 connects to the network 104 and/or connects to the monitoring hubs 100A/100B, and/or sensors 102. In response to a request to admit the user, the server 106 can communicate with server 103 to access historical physiological data of the user previously generated before the user was admitted to the healthcare facility. In some implementations, the server 106 can determine whether historical physiological data on the server 103 originated from a medically approved device. In some implementations, the server 106 can retrieve data from server 103 only if the data was generated by an approved device. In some implementations, the server 106 can retrieve data from the server 103 whether the data was generated from an approved or unapproved device. In some implementations, the server 106 can characterize data from the server 103 depending on whether it originated from an approved device. For example, the server 106 can mark the data with metadata to indicate if it was generated by an approved device or an unapproved device. In some implementations, the server 106 can access historical physiological data stored on the user device 107 directly from the user device 107 such as over network 104.

Advantageously, when a user is admitted for physiological monitoring with PMS 150, the server 106 can access historical physiological data collected before the user was admitted for monitoring by the PMS 150. This historical physiological data (such as data generated when a user was at their home), can serve as important contextual information which can improve physiological monitoring of the user with the PMS 150 moving forward. For example, the PMS 150 can process real-time physiological data generated by the PMS 150 in combination with the historical physiological data which can result in generating more accurate physiological parameters, more accurate notifications, and overall more accurate data processing. Moreover, the PMS 150 can display real-time data generated by the PMS 150 in combination with historical physiological data generated before the user was admitted for monitoring in the healthcare environment. To illustrate the benefits of providing historical physiological data to the server 106 an example is provided. In this example, the user is going about their ordinary daily routine (at home, at work, etc.) while the server 103 is collecting physiological data originating from the sensor 102D during some time frame (for example, hours, days, weeks, etc.). During this time frame the user experiences one or more cardiac arrhythmias which the user may or may not have noticed but which were captured in the physiological data collected and stored at server 103. Eventually, the user is admitted to a healthcare facility for treatment such as in response to a cardiac arrest. The server 106 retrieves the historical physiological data from the server 103 (and/or directly from user device 107) which includes all the physiological data relating to the cardiac arrythmias experienced by the user during the time frame before being admitted for treatment. The user can now be treated at the healthcare facility with a much more complete set of data spanning a greater length of time and capturing important information.

The user device 107 can communicate with any of the devices of PMS 150 such as the server 106, the sensors 102, and/or the monitoring hubs 100. In some implementations, the user device 107 can act as a monitoring hub during physiological monitoring with the PMS 150. For example, the user device 107 can receive data originating from sensors 102A-102C (which can include data processed by server 106) and can display indicia of said data on a display of user device 107. In some implementations, the user device 107 can participate in a physiological transfer process. For example, physiological monitoring can be transferred from monitoring hub 100A/B to user device 107 and/or physiological monitoring can be transferred from user device 107 to monitoring hub 100A/B. In some implementations, the user device 107 or any features thereof can be or can be incorporated into a wearable system as described herein.

The user device 107 can receive physiological data originating from sensors 102A-102C periodically, upon request, and/or in response to one or more conditions. For example, the server 106 may communicate data to the user device 107 in response to a patient being discharged from a healthcare facility. Such data can include historical physiological data previously collected from sensors 102A-102C and communicated to the server 106 from monitoring hub 100A and/or 100B. Advantageously, the user may have access to physiological data collected while the patient was in the healthcare facility which may facilitate continuing to care for the user when the user departs the healthcare facility. Such data can also include communication data to allow the user device 107 to wirelessly connect with a sensor, such as sensor 102D. For example, when a user is discharged from a healthcare facility, the sensor 102D can be provided to the user to continue monitoring the user after they depart with the user device acting as the monitoring hub. Sensor 102D may or may not have been used to monitor the user when they were in the healthcare facility. In some implementations, the server 106 can provide a monitoring protocol to the user device 107 associated with sensor 102D. The monitoring protocol can indicate to the user how to use the sensor 102D (for example, when to take measurements). The server 106 may verify the user device 107 is associated with the user before communicating patient-specific data to the user device 107.

Advantageously, after a user departs a healthcare facility, the user device 107 can have access to historical physiological data generated while the user was being monitored at the healthcare facility. The user device 107 can process real-time data moving forward in light of historical physiological data which can lead to generating more accurate parameters, notifications, etc. Moreover, the user device 107 can cause real-time data to be displayed in combination with historical data. The user device 107 can choose to continue to communicate physiological data to the server 106 after the user departs the healthcare facility. Such data can be accessed by healthcare providers which can improve remote healthcare.

FIG. 1B is a schematic block diagram illustrating an additional example implementation of the physiological monitoring system (PMS) 150. The example implementation shown in FIG. 1B may result from a request to transfer physiological monitoring from monitoring hub 100A to monitoring hub 100B. For example, as shown in the example implementation of FIG. 1A, the sensor(s) 102 may be in communication with the monitoring hub 100A and may not be in communication with the monitoring hub 100B. The PMS 150 can transfer physiological monitoring (for example, in response to a user request) from the monitoring hub 100A to the monitoring hub 100B. As shown in FIG. 1B, after the PMS 150 has transferred the physiological monitoring from monitoring hub 100A to monitoring hub 100B, the sensor(s) 102 may be in communication with the monitoring hub 100B and may not be in communication with the monitoring hub 100A. The monitoring hub 100B can receive and/or display physiological data received from the sensor(s) 102 via the wireless communication connection (for example, Bluetooth and/or other wireless communication protocol) established with the sensor(s) 102 as a result of the transfer.

The monitoring hubs 100A, 100B can receive a user input 108. The user input 108 can include identification data and/or a transfer request. The user input 108 can be a manual user input such as via a display of the monitoring hubs 100A, 100B or via one or more buttons of the monitoring hubs 100A, 100B. For example, a user may press a button of the monitoring hubs 100A, 100B to request a transfer. The user input 108 can include an electronic input such as an electronic signal generated in response to a wireless communication protocol. For example, a user may bring a communication device (for example, user ID badge) in proximity to the monitoring hubs 100A, 100B to generate an electronic signal (for example, via NFC and/or RFID) at the monitoring hubs 100A, 100B.

In some implementations, the monitoring hub 100A can optionally communicate with the monitoring hub 100B. In some implementations the monitoring hub 100A may communicate with the monitoring hub 100B via a wireless communication protocol such as WiFi, Bluetooth, near field communication (NFC), radio frequency identification (RFID), cellular, 1G, 2G, 3G, 4G, 5G, and/or Zigbee. The monitoring hub 100A may communicate data to the monitoring hub 100B and/or receive data from the monitoring hub 100B including communication data (for example, device addresses of the sensors 102), physiological data, identification data (for example, user ID), transfer requests, request approval status, or the like. In some implementations, the monitoring hub 100A may communicate with the monitoring hub 100B only while the PMS 150 is transferring the physiological monitoring from the monitoring hub 100A to the monitoring hub 100B. For example, in some implementations, the monitoring hub 100A may only communicate with the monitoring hub 100B until the transfer is complete, the monitoring hub 100B has established communication with the sensor(s) 102, or the like. In some implementations, the monitoring hub 100A may communicate with the monitoring hub 100B to facilitate the transfer (for example, may transmit communication data to facilitate establishing communication between the monitoring hub 100B and the sensor(s) 102. In some implementations, the monitoring hub 100A may not communicate with the monitoring hub 100B. In some implementations, the monitoring hub 100B may receive data from the server 106, such as wireless configuration data and/or physiological data which may facilitate transferring physiological monitoring to the monitoring hub 100B. In some implementations, physiological monitoring can be transferred to and/or from user device 107. For example, user device 107 can act as monitoring hub 100A and/or monitoring hub 100B.

As shown in FIGS. 1A-1B, the server 106 can receive audio data from monitoring hub 100A and/or monitoring hub 100B such as via one or more wireless communication protocols (for example, WiFi) over network 104. For example, monitoring hub 100A and/or 100B may detect audio with one or more microphones and may communicate the detected audio to the server 106. In some implementations, monitoring hub 100A and/or 100B may detect (for example, continuously) ambient noise and may communicate the ambient noise to the server 106. In some implementations, the monitoring hub 100A and/or 100B may communicate processed and/or unprocessed audio data to the server 106, such as raw acoustic signals detected with a microphone and/or representations of acoustic signals such as decibel levels detected. Advantageously, in some implementations, the monitoring hub 100A and/or 100B may only communicate decibel levels of detected audio (for example, without communicating underlying audio signals) to the server which may allow the server to access information relating to general noise level in an environment while preventing the server from accessing sensitive information, such as audio data of a conversation.

The monitoring hubs 100A and 100B may be at different locations from each other and may change locations as they travel about an environment, such as a healthcare facility. As the monitoring hubs 100A and 100B travel throughout an environment they can detect and communicate (for example, continuously) audio data to the server 106 that they detect with microphone(s). The server 106 can access audio data detected by and/or communicated from monitoring hub 100A and/or 100B. The server 106 can process the audio data to determine an audio map of an environment. An audio map can indicate noises and/or noise levels associated with locations of an environment, such as particular rooms, hallways, floors, etc. of a building. For example, an audio map can indicate that a particular location, such as room, has an average decibel level. As another example, an audio map can indicate that a particular location generally has particular sounds such as alarms, people speaking, equipment operating, etc. An audio map may represent historical audio-spatial data, current audio-spatial data, and/or predictive future audio-spatial data. The server 106 can determine a location for a patient based on an audio map generated using audio data from the monitoring hub 100A and/or 100B as well as patient characteristics such as health condition, age, diagnosis, scheduled procedure, medication regimen, or the like. For example, the server 106 can determine that a patient having a certain health condition should be in a location with a low noise level (for example, below a certain decibel threshold) to improve their health and the server 106 can determine a location that has historical met, is currently meeting, or is predicted to meet that noise level criteria. In some implementations, the server 106 can determine locations for patients based on decibel level and/or noise types. For example, the server 106 can determine that a patient should avoid being in certain locations having frequent alarms or frequent human conversations because these types of noise may be particularly disturbing to a patient (although perhaps below a certain decibel threshold), whereas a constant low-frequency noise from a machine that is operating may not disturb a patient (although perhaps above a decibel threshold).

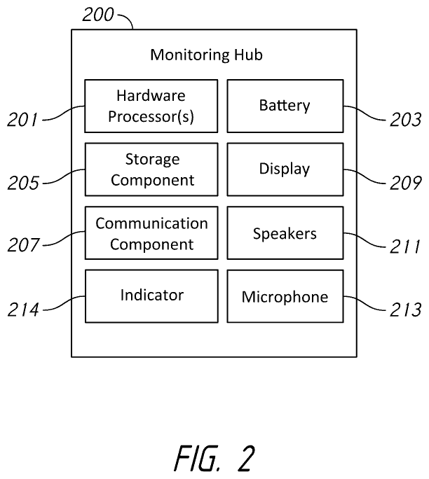

FIG. 2 is a block diagram illustrating an example implementation of a monitoring hub 200. The monitoring hub 200 can include similar structural and/or operational features as any of the other example monitoring hubs shown and/or discussed herein such as monitoring hubs 100A, 100B discussed in FIG. 1A. In some implementations, monitoring hub 200 can be a user device such as a watch or phone.

As shown, the monitoring hub 200 can include a hardware processor 201, a storage component 205, a communication component 207, and a battery 203. The hardware processor 201 can be configured, among other things, to process data, execute program instructions to perform one or more functions, and/or control the operation of the monitoring hub 200. For example, the hardware processor 201 can process physiological data obtained from physiological sensors and can execute instructions to perform functions related to storing and/or transmitting such physiological data. As another example, the hardware processor 201 can process data relating to transfer requests, identification data, and/or transfer approval status.

The storage component 205 can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (for example, CD-ROM, DVD-ROM, etc.), magnetic disks (for example, hard disks, floppy disks, etc.), memory circuits (for example, solid state drives, random-access memory (RAM), etc.), and/or the like. The storage component 205 can store data including processed and/or unprocessed physiological data originating from physiological sensors. The storage component 205 can store data including communication data such as link keys and/or device addresses associated with sensors and/or monitoring hubs. The storage component 205 can store persistent data and/or non-persistent data. Persistent data may be data that is preserved (for example, not deleted from storage) when the computing system is powered down and/or when an application is terminated. Persistent data may be stored for longer than 30 seconds, longer than 60 seconds, longer than 5 minutes, longer than 10 minutes, longer than 30 minutes, longer than 1 hour, longer than 6 hours, longer than 12 hours, longer than 24 hours, or the like. Non-persistent data may be data that is deleted or otherwise lost when the computing system is powered down and/or when an application is terminated. Non-persistent data may be stored for less than 24 hours, less than 12 hours, less than 6 hours, less than 1 hour, less than 30 minutes, less than 10 minutes, less than 5 minutes, less than 60 seconds, less than 30 seconds, less than 20 seconds, less than 10 seconds, less than 5 seconds, less than 1 seconds, or the like. Non-persistent data may be stored for a shorter period of time than persistent data. In some implementations, persistent data may be stored in non-volatile memory. In some implementations, non-persistent data may be stored in volatile memory such as RAM. The storage component 205 can store data in a buffer. A buffer may store data for a period of time before deleting the data. The period of time can be fixed. The buffer may automatically delete data stored therein upon expiration of a period of time. The period of time may be between about 0.01 seconds and 0.15 seconds, between 0.1 seconds and 1.5 seconds, between 1 second and 5 seconds, between 1 second and 10 seconds, between 10 seconds and 60 seconds, between 30 seconds and 60 seconds, between 1 minute and 3 minutes, between 1 minute and 5 minutes, between 1 minute and 10 minutes, between 5 minutes and 30 minutes, between 20 minutes and 60 minutes, or greater than 60 minutes.

The communication component 207, which may also be referred to as a communication system, can facilitate communication (via wired and/or wireless connection) between the monitoring hub 200 (and/or components thereof) and separate computing devices, such as separate monitoring hubs, monitoring devices, sensors, systems, servers, or the like. For example, the communication component 207 can be configured to allow the monitoring hub 200 to wirelessly communicate with other devices, systems, using any combination of a variety of communication protocols and/or over one or more networks. The communication component 207 can be configured to implement any combination of a variety of wireless communication protocols, such as Wi-Fi (802.11x), Bluetooth®, ZigBee®, Z-wave®, cellular telephony, infrared, near-field communications (NFC), radio frequency identification (RFID), satellite transmission, proprietary protocols, combinations of the same, and the like. The communication component 207 can allow data and/or instructions to be transmitted and/or received to and/or from the monitoring hub 200 and separate computing devices. The communication component 207 can be configured to transmit and/or receive (for example, wirelessly) processed and/or unprocessed physiological data with separate computing devices including physiological sensors, other monitoring hubs, remote servers, or the like. As another example, the communication component 207 can be configured to transmit and/or receive (for example, wirelessly) communication data (for example, link keys and/or device addresses associated with monitoring hubs and/or sensors) with separate computing devices including physiological sensors, other monitoring hubs, remote servers, or the like. The communication component 207 can be embodied in one or more components that may be in communication with each other. The communication component 207 can include one or more wireless transceivers, one or more antennas, one or more radios, and/or a near field communication (NFC) component such as a transponder. The communication component 207 can wirelessly communicate or connect to one or more remote computing devices over a network such as by implementing one or more wireless communication protocols.

The monitoring hub 200 can include a battery 203. The battery 203 can provide power for hardware components of the monitoring hub 200 described herein. The battery 203 can be, for example, a lithium battery. Additionally or alternatively, the monitoring hub 200 can be configured to obtain power from a power source that is external to the monitoring hub 200. For example, the monitoring hub 200 can include or can be configured to connect to a cable which can itself connect to an external power source to provide power to the monitoring hub 200.

The monitoring hub 200 can include a display 209. The display 209 can include an LED screen, an LCD screen, an OLED screen, a QLED screen, a plasma display screen, a quantum dot display screen, or the like. The display 209 may be responsive to touch. For example, the display screen may comprise a touchscreen such as a resistive touchscreen, a capacitive touchscreen, an infrared touchscreen, a surface acoustic wave touchscreen, or the like. The display 209 can receive user input. The display 209 can render one or more user interfaces. The display 209 can display indicia of physiological data.

The monitoring hub 200 can include one or more speakers 211. The speakers 211 can emit an audio signal. The speakers 211 can emit an alarm. The speakers 211 can emit a voice audio signal. The speakers 211 can include a plurality of speakers positioned apart from one another at various positions on the monitoring hub 200. The speakers 211 can emit stereophonic audio. The speakers 211 can emit audio using one or more audio channels. The speakers 211 can emit audio in a plurality of directions. The speakers 211 can emit monaural audio. The speakers 211 can emit audio originating during a voice call and/or video call.

The monitoring hub 200 can include one or more microphones 213. The microphone 213 can detect audio signals and generate signals responsive to the detected audio signals. The microphone 213 can detect ambient noise. The microphone 213 can detect a noise level in an environment surrounding the monitoring hub 200. The microphone 213 can detect a noise level in an environment adjacent to and/or encompassing a subject. The monitoring hub 200 can adjust one or more operations based on at least an ambient noise level detected by the microphone 213. The monitoring hub 200 can perform one or more operations to increase a patient's comfort based on at least an ambient noise level detected by the microphone 213. The microphone 213 can detect the voice a person speaking. The microphone 213 can detect a person's voice during a voice call and/or during a video call. In some implementations, the monitoring hub 200 can include two microphones 213. One of the microphones can detect ambient noise and another of the microphones 213 can detect a person's voice. The hardware processor 201 can perform noise cancelling based on audio detected by the microphones 213. For example, the hardware processors 201 may cancel (for example, suppress, subtract, reduce, etc.) ambient noise detected with one microphone from audio (for example, a person's voice) detected with another microphone, which may greatly improve the audio quality of the person's voice such as during audio calls with the monitoring hub 200. In some implementations, the monitoring hub 200 can communicate audio detected with the microphone(s) 213 to a remote computing device via communication component 207. For example, the monitoring hub 200 (with communication component 207) can communicate audio detected with the microphone 213 to a remote computing device (for example, another monitoring hub, phone, laptop, etc.) during an audio call. As another example, the monitoring hub 200 can communicate audio detected with the microphone 213 to a remote server which may determine an audio map of an environment from the audio communicated from the monitoring hub 200 and/or from other monitoring hubs. In some implementations, the microphone 213 may continuously monitor an ambient noise and may communicate the ambient noise to a remote server.

The monitoring hub 200 can implement a voice call. The monitoring hub 200 can connect to one or more cellular devices. The monitoring hub 200 can implement a voice call using cellular telephony such as via the communication component 207. The monitoring hub 200 can implement a video call. A patient being monitored by the monitoring hub 200 can talk to another person in a remote location, such as a caregiver, via the monitoring hub 200 which can implement one or more wireless communication protocols, such as mobile telephony, to connect to one or more remote computing devices over a network, and which can detect the patient's voice using the microphone 213.

The monitoring hub 200 can include one or more indicators 214. The indicator 214 can include a visual indicator. The indicator 214 can include an LED indicator, comprising one or more LEDs. The indicator 214 can emit one or more visual signals. The visual signals may correspond to a physiological status of a patient being monitored by the monitoring hub 200. The indicator 214 can emit visual signals including a plurality of colors. The indicator 214 can emit visual signals according to a color-code scheme wherein various colors may correspond to various physiological statuses of a patient being monitored by the monitoring hub 200.

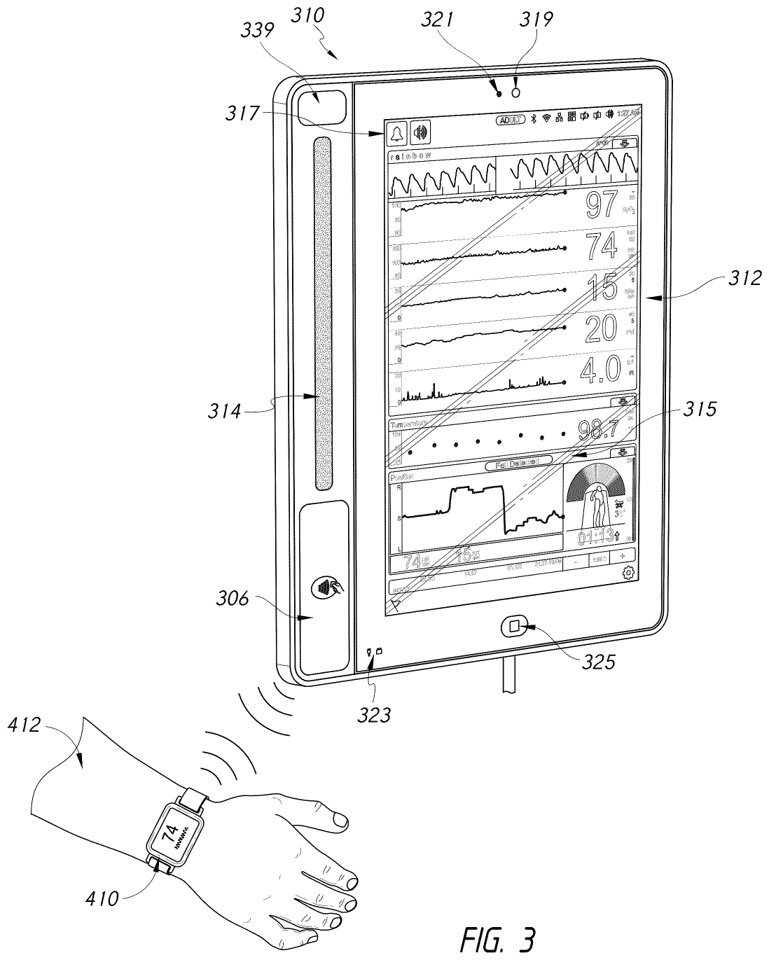

FIG. 3 illustrates an example method for transferring physiological monitoring between monitoring hub 310 and user device 410. In the example shown user device 410 is a watch. In some implementations, user device 410 can be another device such as a phone. User device 410 can include one or more physiological sensors configured to generate physiological data of the user 412. User device 410 can include a display configured to display indicia of physiological data, such as physiological parameters. User device 410 can store physiological data.

Monitoring hub 310 can include similar structural and/or operational features as any of the other example monitoring hubs shown and/or described herein.

The monitoring hub 310 can include a communication interface 306. The communication interface 306 can include electronics configured to execute a wireless communication protocol. The communication interface 306 can include an NFC and/or RFID transponder or reader. The communication interface 306 can include a bar code reader or scanner. The communication interface 306 can include a QR code reader or scanner. The communication interface 306 can include a fingerprint scanner. The communication interface 306 can include a camera configured to capture images of a user's face for facial recognition. The communication interface 306 can use magnetic field induction to communicate with a separate device. The communication interface 306 can communicate with a user device such as a user ID badge, a user phone, a user mobile device, a user smartwatch, or the like, to identify and/or verify a user such as by receiving a unique user identification from the user device.

Monitoring hub 310 can include a light sensor 319, a microphone 321, one or more indicators 323, a display 312, and/or a button 325. The light sensor 319 can be configured to detect an ambient light. The monitoring hub 310 may change a display brightness of the display 312 based on the ambient light detected by the light sensor 319. In some implementations, the light sensor 319 can comprise a camera configured to capture images. The microphone 321 can be configured to detect sound. In some implementations, the monitoring hub 310 may receive user input such as a transfer request as a voice command via a microphone 321. In some implementations, the monitoring hub 310 may implement voice recognition on sounds detected by the microphone 321. The indicator(s) 323 may include one or more LEDs. The indicator(s) 323 may indicate a status and/or operational state of the monitoring hub 310 such as power level, state of wireless connection, or the like. A user may operate the button 325 to control operation of the monitoring hub 310.

In this example implementation, the monitoring hub 310 is in alarm mode. During alarm mode, a status indicator 314 may illuminate (for example, red). During alarm mode, a display 312 of the monitoring hub 310 may display one or more badges, icons, banners, symbols, indicators, or the like to indicate the monitoring hub 310 is in alarm mode. During this example alarm mode, the display 312 can include a “Fall Detected” banner 315. During this example alarm mode, the display 312 illuminates an alarm icon 317.

In some implementations, monitoring hub 310 can include an alarm toggle button 339. A user may press the alarm toggle button 339 to silence an alarm. A user may press the alarm toggle button 339 to change a state of an alarm. The alarm toggle button 339 can comprise a capacitive sensor. The alarm toggle button 339 can comprise one or more mechanical actuators.

Physiological monitoring can be transferred between user device 410 and monitoring hub 310. For example, monitoring hub 310 can be an origin monitoring hub that displays indicia of physiological data originating from sensors coupled to user 412. Physiological monitoring can be transferred from monitoring hub 310 to the user device 410 which can act as a destination monitoring hub. In some implementations, the user 412 can be a patient being monitored by the monitoring hub 310 within a healthcare environment. The user 412 can be discharged from the healthcare environment to return home. The user device 410 can act as the destination monitoring hub to continue monitoring the user 412 after the user departs the healthcare environment. For example, the user device 410 can communicate with sensors coupled to the user 412 (which may be the same or different sensors communicating data to the monitoring hub 310) and can display indicia of physiological data originating from the sensors. As another example, the user device 410 can act as the origin monitoring hub such as before a user is admitted to a healthcare environment (for example, when a user is at home and/or going about their ordinary daily life). The user device 410 can display indicia of physiological data originating from sensor coupled to the user 412 which may be integrated with the user device 410. Physiological monitoring can be transferred from the user device 410 to the monitoring hub 310 such as when the user 412 is admitted to a healthcare environment as a patient. The monitoring hub 310 can continue monitoring the user 412 as the destination monitoring hub and can have access to historical physiological data originating from the user device 410. For example, the monitoring hub 310 can display indicia of real-time physiological data in combination with historical physiological data previously collected by the user device 410.

Transferring physiological monitoring between monitoring hub 310 and user device 410 can occur in response to wireless communication between the monitoring hub 310 and user device 410. For example, when the monitoring hub 310 and user device 410 are brought within spatial proximity of each other the monitoring hub 310 and user device 410 can communicate with each other via a wireless communication protocol such as NFC which can initiate transferring physiological monitoring. In some implementations, the user device 410 can communicate identification data to the monitoring hub 310 (for example, via NFC) to identify the user 412. The user device 410 can store identification data in data storage such as in EPROM and/or EEPROM. Transferring physiological monitoring either to the user device 410 or from the user device 410 can occur in response to verifying the identification data. For example, when transferring physiological monitoring from the monitoring hub 310 to the user device 410, the monitoring hub 310 can access identification data from the user device 410 (for example, via NFC) to verify that the identity of the user 412 corresponds with an identity of a patient (for example, stored in electronic medical records of a hospital server) being monitored by the monitoring hub 310 to ensure that monitoring occurs for the same person when monitoring is transferred between devices. In some implementations, the identification data can indicate whether a user has authority (or has given authorization) to transfer physiological monitoring. As another example, when transferring physiological monitoring from the user device 410 to the monitoring hub 310, the monitoring hub 310 can access identification data from the user device 410 to store such that future physiological data monitored by the monitoring hub 310 can be associated with the user identification data. In some implementations, the monitoring hub 310 (or other associated computing device) can verify that user 412 has given their authorization to transfer physiological monitoring from the user device 410 (along with historical physiological data) based on identification data. In some implementations, transferring physiological monitoring may not occur if the user 412 has indicated (for example, via their identification data) that they do not authorize transferring physiological monitoring (for example, from the user device 410).

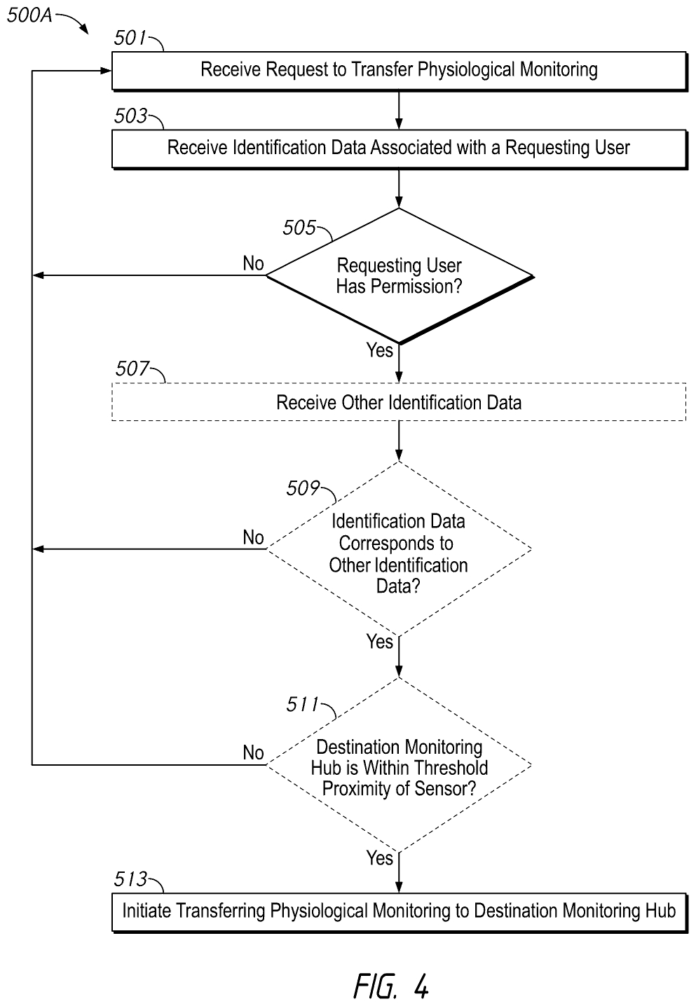

FIG. 4 is a flowchart illustrating an example method 500A of transferring physiological monitoring from an origin monitoring hub to a destination monitoring hub. One or more hardware processors can execute method 500A, or portions thereof. Method 500A, or portions thereof, can be implemented on one or more computing devices described herein, such as an origin monitoring hub, a destination monitoring hub, a server, etc. Method 500A, or portions thereof, may be executed by one or more hardware processors of a single computing device. Method 500A, or portions thereof, may be executed by one or more hardware processors of multiple computing devices such as computing devices that are remote to each other and/or in wireless communication with each other. In some implementations, one or more hardware processors associated with a server, such as server 106 shown and/or described herein, may execute method 500A, or portions thereof. Method 500A is provided as an example and is not intended to be limiting of the present disclosure. In some implementations, one or more hardware processors executing the method 500A may omit portions of the method 500A, may add additional operations, and/or may rearrange an order in which the operations of the method 500A are executed.

At block 501, one or more hardware processors can receive a request to transfer physiological monitoring from one monitoring hub (for example, origin monitoring hub) to another monitoring hub (for example, destination monitoring hub). In some implementations, the origin monitoring hub and/or the destination monitoring hub may receive the request to transfer such as via a button press on the monitoring hub, voice commands, NFC, or other user input. In some implementations, the method 500 may not include block 501. For example, the computing device(s) may receive identification data as discussed at block 503 without receiving a request to transfer at block 501. In some implementations, the request to transfer physiological monitoring may comprise a request to establish initial physiological monitoring at a monitoring hub without terminating physiological monitoring at another monitoring hub such as if physiological monitoring is being established for the first time or if physiological monitoring has not occurred recently prior to the request.

At block 503, the one or more hardware processors can receive identification data. The identification data may be associated with a user requesting the transfer. The one or more hardware processors may receive the identification data via a monitoring hub such as an origin monitoring hub and/or a destination monitoring hub. The identification data may be associated with a monitoring hub at which it received. In some implementations, the origin monitoring hub may receive the identification data, for example, as shown and/or described herein. In some implementations, receiving the identification data may serve as receiving the request to transfer physiological monitoring described at block 501. For example, method 500A may not implement block 501 or may implement block 501 as part of block 503.

At decision block 505, the one or more hardware processors may determine whether the user requesting to transfer physiological monitoring has appropriate permission to perform the transfer. In some implementations, the one or more hardware processors may determine whether the requesting user has permission based on at least the identification data received at block 503.

In some implementations, the one or more hardware processors may determine whether the requesting user has appropriate permission based on one or more of an identification of the user, job title of the user, role of the user, task assigned to the user, or the like, which may be determined by the identification data. For example, a doctor (for example, as identified by a user ID include in the identification data) may have permission to perform the transfer whereas a nurse may not. As another example, a certain type of doctor (for example, cardiologist) may have permission to perform the transfer whereas another type of doctor (for example, surgeon) may not. As another example, a healthcare provider assigned to a patient may have permission to perform the transfer whereas a healthcare provider not assigned to the patient may not.

In some implementations, the one or more hardware processors may determine whether the requesting user has appropriate permission based on a time of the request. For example, a user may not have permission to transfer physiological monitoring between monitoring hubs while a subject being monitored is undergoing surgery, or sleeping, or during a scheduled meal time, or the like. In some implementations, the one or more hardware processors may determine whether the requesting user has appropriate permission based on a reason provided by the user which may be inputted at a monitoring hub by the user (for example, as part of identification data). In some implementations, the one or more hardware processors may determine whether the requesting user has appropriate permission based on a location of the subject being monitored and/or a location of monitoring hub. For example, a user may not have permission to transfer physiological monitoring from a monitoring hub that is stationed in a particular hospital room to a mobile monitoring hub if the subject is supposed to remain in the particular hospital room. As another example, a user may not have permission to transfer physiological monitoring from a mobile monitoring hub to a monitoring hub that is stationed in a particular hospital room (for example, surgical operating room) if the subject is not supposed to be in the particular hospital room (for example, the subject is not scheduled for surgery).

Advantageously, verifying permissions associated with a user can improve quality of health care such as by ensuring that a PMS only transfers physiological monitoring under appropriate circumstances which can ensure that a subject is receiving proper healthcare (for example, a subject is not relocated to a different room in a hospital if not appropriate).

In response to determining that the requesting user has permission to perform the transfer, the one or more hardware processors may proceed to block 507. In response to determining that the requesting user does not have permission to perform the transfer, the one or more hardware processors may return to block 501.

At block 507, the one or more hardware processors can optionally receive other identification data. The other identification data may be associated with a user requesting the transfer. The one or more hardware processors may receive the other identification data via a monitoring hub such as an origin monitoring hub and/or a destination monitoring hub. The other identification data may be associated with a monitoring hub at which it received. In some implementations, the destination monitoring hub may receive the other identification data, for example, from the same user the same way the origin monitoring hub received identification data. For example, a user can swipe an ID badge at both origin and destination monitoring hubs, wherein said ID badge is readable via NFC at the monitoring hubs.

At decision block 509, the one or more hardware processors can determine whether the identification corresponds to the other identification data. The one or more hardware processors may compare the identification data. For example, the one or more hardware processors may compare identification data received by an origin monitoring hub with identification data received by a destination monitoring hub. In some implementations, comparing identification data can include determining whether the identification data received at the origin monitoring hub corresponds with identification data received at the destination monitoring hub. In some implementations, comparing identification data can include determining whether the identification data received at the origin monitoring hub matches identification data received at the destination monitoring hub. In some implementations, comparing identification data can include comparing user identifications included in the identification data.

In some implementations, the one or more hardware processors may determine that identification data correspond if the identification data (or portions thereof) in each of the respective identification data match each other, such as if they are identical or substantially similar. This may indicate for example that the user requesting transfer at the origin monitoring hub is the same user requesting transfer at the destination monitoring hub. In some implementations, the one or more hardware processors may determine that identification data do not correspond if the identification data (or portions thereof) in each of the respective identification data do not match each other. This may indicate that the user requesting transfer at the origin monitoring hub is not the same user requesting transfer at the destination monitoring hub.

In some implementations, the one or more hardware processors may determine that identification data correspond if the respective identification are associated with each other. This may indicate that a user requesting transfer at the origin monitoring hub is associated with the user requesting transfer at the destination monitoring hub. For example, a user requesting transfer at an origin monitoring hub may be associated with a user requesting transfer at a destination monitoring hub if the users are working together (for example, providing healthcare services at a same time to a patient being monitored by the PMS). In some implementations, the one or more hardware processors may determine that a first user's identification is associated with a second user's identification if the first and second users are within the same group, for example, healthcare providers in the same or a similar group and/or location (such as a floor or a care unit).

In some implementations, the one or more hardware processors may determine that identification data correspond if they are received (for example, at blocks 503 and 507) within a threshold time. For example, a request to transfer physiological monitoring at an origin monitoring hub may “time out” if a user fails to make a corresponding request at a destination monitoring hub.

Advantageously, comparing the identification data (for example, that they correspond) can improve fidelity of transferring physiological monitoring from one monitoring hub to a proper monitoring hub. For example, determining that identification data correspond to each other may ensure that the PMS transfers physiological monitoring to the correct monitoring hub rather than to an incorrect monitoring hub which may have identification data that does not correspond to the identification data received at the origin monitoring hub. Advantageously, verifying that identification data correspond can facilitate accurately transferring physiological monitoring between desired monitoring hubs in a PMS that includes numerous monitoring hubs and/or that includes numerous requests to transfer physiological monitoring between various monitoring hubs occurring at or near the same time. For example, by verifying that identification data of origin and destination monitoring hubs correspond, a PMS can accurately transfer physiological monitoring between appropriate pairs of monitoring hubs (for example, between origin and destination monitoring hubs) at a same or similar time such as between monitoring hub pair A, between monitoring hub B, and between monitoring hub pair C, without incorrectly transferring physiological monitoring between the hubs of different pairs (for example, from a hub in pair A to a hub in pair B).

In some implementations, in response to determining that the identification data correspond, the one or more hardware processors may proceed to block 511. In some implementations, in response to determining that the identification data do not correspond, the one or more hardware processors may return to block 501.

At decision block 511, the one or more hardware processors can optionally determine whether a destination monitoring hub is within a threshold proximity of a sensor. The one or more hardware processors may determine the proximity of a monitoring hub to a sensor based on at least a wireless signal strength between the monitoring hub and the sensor. In response to determining that the destination monitoring hub is within a threshold proximity of the sensor, the one or more hardware processors may proceed to block 513. In response to determining that the destination monitoring hub is not within a threshold proximity of the sensor, the one or more hardware processors may return to block 501.

At block 513, the one or more hardware processors can initiate transferring physiological monitoring to the destination monitoring hub. Transferring physiological monitoring can include establishing wireless communication between one or more sensors and a destination monitoring hub. Transferring physiological monitoring can include terminating wireless communication between one or more sensors and an origin monitoring hub. In some implementations, terminating communication with origin monitoring hub may precede establishing communication with destination monitoring hub. In some implementations, terminating the communication may occur automatically as a result of establishing the communication. Transferring physiological monitoring can include transferring physiological monitoring associated with all of the sensors in communication with the origin monitoring hub to the destination monitoring hub. Transferring physiological monitoring can include transferring physiological monitoring associated with less than all of the sensors in communication with the origin monitoring hub to the destination monitoring hub.

Advantageously, method 500A can provide a system for transferring physiological monitoring from one monitoring hub to another monitoring hub (for example, establishing and/or terminating communication between monitoring hubs and sensors) without having to unplug, plug, and/or replug cables, wiring, etc. of the monitoring hubs and/or physiological sensors.

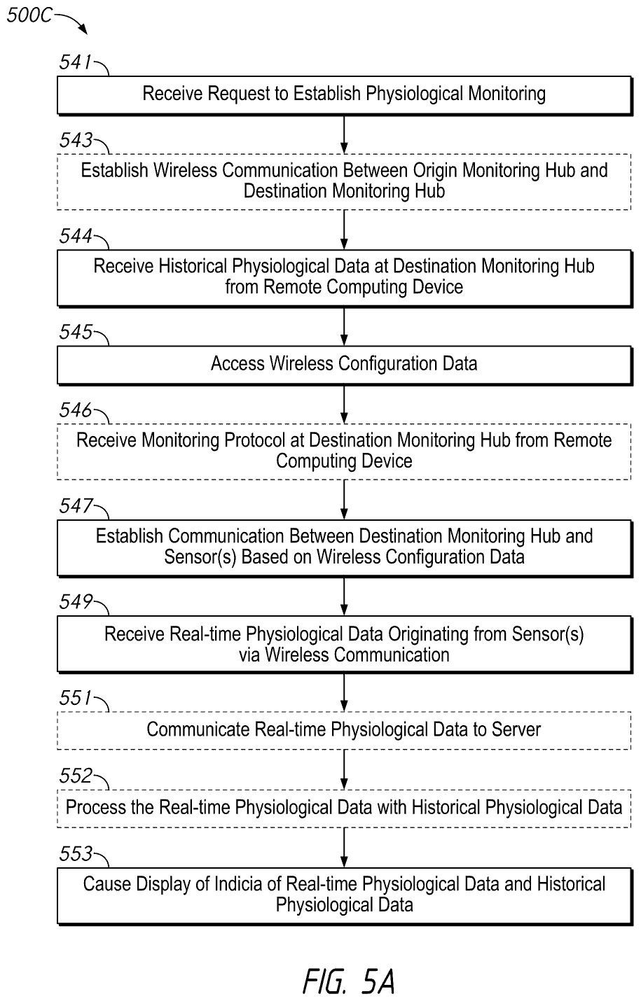

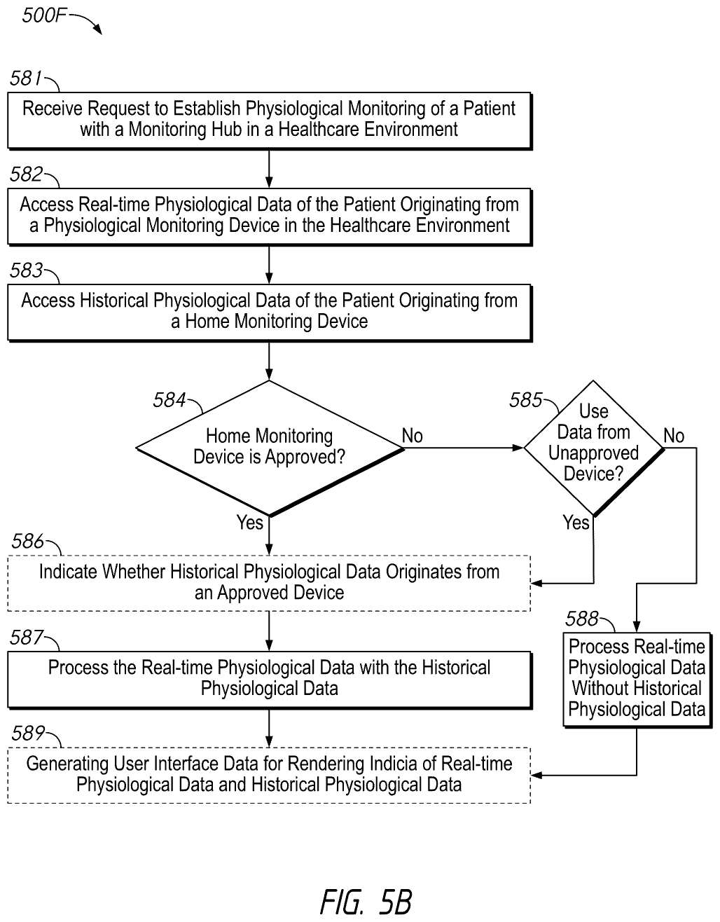

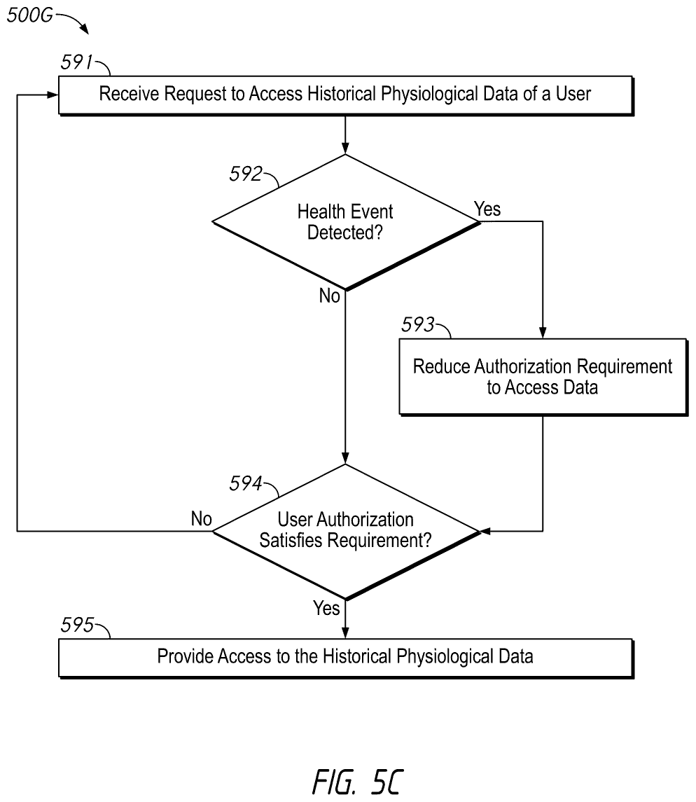

FIG. 5A is a flowchart illustrating an example method 500C associated with transferring physiological monitoring from an origin monitoring hub to a destination monitoring hub. One or more hardware processors can execute method 500C, or portions thereof. Method 500C, or portions thereof, can be implemented on one or more computing devices described herein, such as an origin monitoring hub, a destination monitoring hub, a server, a user device, etc. Method 500C, or portions thereof, may be executed by one or more hardware processors of a single computing device. Method 500C, or portions thereof, may be executed by one or more hardware processors of multiple computing devices such as computing devices that are remote to each other and/or in wireless communication with each other. In some implementations, one or more hardware processors associated with a destination monitoring hub may execute method 500C, or portions thereof. Method 500C is provided as an example and is not intended to be limiting of the present disclosure. In some implementations, one or more hardware processors executing the method 500C may omit portions of the method 500C, may add additional operations, and/or may rearrange an order in which the operations of the method 500C are executed.

At block 541, one or more hardware processors may receive a request to establish physiological monitoring at a destination monitoring hub. The request to establish physiological monitoring at the destination monitoring hub may be included as part of a request to transfer physiological monitoring from an origin monitoring hub to the destination monitoring hub. The request can be associated with a patient changing environments, such as being admitted to a healthcare facility, changing rooms within a healthcare facility, or being discharged from a healthcare facility. The request to establish physiological monitoring may indicate one or more sensors with which the destination monitoring hub is to establish wireless communication. The one or more hardware processors may receive the request via one or more monitoring hubs such as shown and/or described herein. In some implementations, the one or more hardware processors may additionally verify a permission of a requesting user such as based on at least identification data.

At block 543, the one or more hardware processors can optionally establish a wireless communication between the origin monitoring hub and the destination monitoring hub. The wireless communication can include a Bluetooth connection. Establishing the wireless communication can include implementing one or more of a pairing process, inquiry process, discovery process, advertising process, paging process, and/or connection process. In some implementations, the one or more hardware processors may cause the destination monitoring hub to become a slave to the origin monitoring hub in a Bluetooth connection.

At block 544, the one or more hardware processors can receive historical physiological data at the destination monitoring hub from a remote computing device, such as a server, such as server 106 shown and/or described herein. The one or more hardware processors can receive the historical physiological data via one or more wireless communication protocols such as WiFi. The historical physiological data can include physiological data previously generated by the one or more sensors. The historical physiological data can include data generated by the one or more sensors prior to establishing wireless communication between the destination monitoring hub and the one or more sensors at block 547. The historical physiological data can include data communicated from the one or more sensors to an origin monitoring hub, such as prior to establishing wireless communication between the destination monitoring hub and the one or more sensors at block 547. The historical physiological data can include data corresponding to a time frame of less than 25 hours, less than 12 hours, less than 6 hours, less than 1 hour, less than 30 minutes, less than 10 minutes, less than 5 minutes, less than 1 minute, less than 30 seconds, less than 10 seconds, or less than 1 second. The one or more hardware processors can receive the historical physiological data as a single transmission or packet of data. The one or more hardware processors can receive the historical physiological data at a single moment of time. The one or more hardware processors can receive the historical physiological data over a time frame that is shorter than the time frame corresponding to which the historical physiological data was generated by the one or more sensors. As an example, the one or more hardware processors can receive a packet of data comprising the historical physiological data as a single transmission and/or at a single moment of time. In some implementations, the one or more hardware processors can receive the historical physiological data prior to any of the other blocks in method 500C.

In some implementations, the one or more hardware processors can receive data associated with the physiological data. Data associated with the physiological data can include user interface data for rending a user interface comprising display indicia of the physiological data. Data associated with the physiological data can include signals correspond to alarms or alerts generated in response to the physiological data. Data associated with the physiological data can include a status associated with the subject corresponding to the physiological data. As an example, the one or more hardware processors can receive a signal associated with an alarm, alert, status, etc, that may have been generated at the origin monitoring hub based on the physiological data. Accordingly, the destination monitoring hub can continue with a same alarm, alert, status, etc, that was occurring on the origin monitoring hub which can preserve a continuity of physiological monitoring. Moreover, the destination monitoring hub may be able to more rapidly initiate an alarm or alert or show a status at least because the destination monitoring hub may not have to re-process the historical physiological data to determine whether to initiate the alarm or alert or determine the status which may reduce processing time and energy requirement which may improve computational efficiencies as well as physiological healthcare monitoring. In one illustrative example, an origin monitoring hub may have generated an alarm corresponding to a critical patient condition based on at least analyzing physiological data received from sensors. Pursuant to transferring physiological monitoring to a destination monitoring hub, the destination monitoring hub may receive, such as from a server, a signal corresponding to the alarm. The destination monitoring hub may immediately initiate the alarm without having to process (historical) physiological data received from the origin monitoring hub, such as via the server. Accordingly, physiological monitoring of the patient may continue with reduced gaps or discontinuities.

In some implementations, the one or more hardware processors may analyze the historical physiological data received at block 544 to determine one or more physiological statuses or trends in physiological data. The one or more hardware processors may analyze the historical physiological data received at block 544 to generate one or more alarms, alerts, or the like, corresponding to the physiological data. Advantageously, because the one more hardware processors may have access to historical physiological data, such as received at block 544, the one or more hardware processors may be able to more accurately analyze physiological data at least because the one or more hardware processors may have access to more physiological data including historical physiological data and real-time physiological data.

At block 545, the one or more hardware processors can access wireless configuration data. The one or more hardware processors can access the wireless configuration data by receiving the wireless configuration data from a remote computing device such as the origin monitoring hub or a server. In some implementations, the one or more hardware processors may receive the wireless configuration data from the origin monitoring hub directly via the wireless communication established at block 543. In some implementations, the one or more hardware processors may not receive the wireless configuration data from the origin monitoring hub. The one or more hardware processors can receive the wireless configuration data indirectly from the origin monitoring hub via an intermediary device. For example, the one or more hardware processors may receive the wireless configuration data from a server (for example, via WiFi) after server has received the wireless configuration data from the origin monitoring hub. The one or more hardware processors can access the wireless configuration data from memory. For example, the one or more hardware processors can access the wireless configuration data from memory stored on the destination monitoring hub. Wireless configuration data stored in memory may have been previously received from a remote computing device. In some implementations, the one or more hardware processors may receive the wireless configuration data from one or more sensors. In some implementations, the one or more hardware processors may generate at least a portion of the wireless configuration data. For example, the one or more hardware processors may generate and/or receive at least a portion of the wireless configuration data from one or more sensors during a pairing process with the one or more sensors. In some implementations, the one or more hardware processors may not generate and/or receive the wireless configuration data during a pairing process.

At block 546, the one or more hardware processors can optionally receive a monitoring protocol at the destination monitoring hub from a remote computing device. A monitoring protocol can include instructions for monitoring the health of a user. The instructions can be computer executable program instructions to cause a computing device to perform monitoring operations including measuring and displaying physiological information. The instructions can include instructions to a user for taking physiological measurements. As an example, when a user is discharged from a healthcare facility, a monitoring protocol can be provided to a user device (acting as a destination monitoring hub) to facilitate monitoring the user after the user departs the healthcare facility with sensors at the user's home. The remote computing device providing the monitoring protocol can be a server or another monitoring hub.

At block 547, the one or more hardware processors can establish wireless communication between the destination monitoring hub and one or more sensors. The one or more hardware processors can establish wireless communication according to one or more wireless communication protocols. The one or more hardware processors can establish wireless communication according to a Bluetooth communication protocol. Establishing wireless communication can comprise establishing a Bluetooth connection (for example, subsequent to a paging process). The one or more hardware processors can establish wireless communication based on at least the wireless configuration data. The one or more hardware processors can establish wireless communication by initiating a paging process. The one or more hardware processors can establish wireless communication based on at least communicating at least a portion of the wireless configuration data to the one or more sensors. The one or more hardware processors can establish wireless communication without initiating a pairing process and/or an inquiry process. In some implementations, the destination monitoring hub may be considered as bonded to the one or more sensors such as by virtue of having access to the wireless configuration data. In some implementations, the destination monitoring hub may have never previously established wireless communication with the one or more sensors. Establishing communication can include establishing communication between the destination monitoring hub and all of the sensors previously in communication with the origin monitoring hub. Establishing communication can include establishing communication between the destination monitoring hub and less than all of the sensors previously in communication with the origin monitoring hub.

Advantageously, accessing the wireless configuration data at block 545, such as receiving the wireless configuration data from a remote computing device such as a server may facilitate establishing wireless communication such as by eliminating the need to perform a pairing process (which can include an inquiry process), which can take up to 10 seconds to complete and can involve non-trivial data processing and communication between remote devices. Accordingly, accessing the wireless configuration data at block 545 may reduce processing requirements to establish a wireless communication at block 547, which can improve efficiency, reduce the time needed to establish a wireless communication, and reduce processing power required to establish wireless configuration data at block 545 which may improve energy conservation and prolong battery life. Moreover, reducing the time needed to establish wireless communication may reduce data loss. For example, data collected by a physiological sensor may be lost while waiting to establish wireless communication between the sensor and a monitoring hub (such as during a pairing process). Reducing data loss can improve physiological monitoring of the subject which can improve health care provided to the subject. Reducing data loss can improve continuous physiological monitoring of the subject while transferring physiological monitoring between monitoring hubs. For example, the monitoring hubs may continuously monitor the subject with a gap in data resulting during the transfer of less than 10 seconds, less than 5 seconds, less than 1 second, less than 0.5 seconds, less than 0.1 seconds, less than 0.05 seconds, less than 0.01 seconds, or the like. Moreover, eliminating the need to perform a pairing process (for example, by accessing wireless configuration data at block 545) may avoid the need to place a sensor in a discovery mode, or may avoid the inability to establish wireless communication if the sensor is not in discovery mode. Advantageously, a user may be able to establish communication between sensors and a destination hub without having to turn off and/or turn on the sensors, the destination hub, and/or the origin hub. Advantageously, a user may be able to establish communication between sensors and a destination hub without having to change a connectivity state or mode of the sensors, the destination hub, and/or the origin hub.

The one or more hardware processors can establish a wireless communication at block 547 automatically, such as without requiring a user input. For example, the one or more hardware processors may establish wireless communication based on a proximity of the one or more sensors with the destination monitoring hub. The one or more hardware processors can establish a wireless communication at block 547 responsive to a user input. For example, a user may provide input and/or authorization to confirm the one or more hardware processors are to establish wireless communication. In some implementations, the user may provide non-contact user input to initiate establishing wireless communication. Non-contact user input can comprise near field communication (NFC) and/or radio frequency identification (RFID). Non-contact user input can comprise facial recognition, eye recognition, fingerprint recognition, gesture recognition, voice recognition, or the like. Non-contact user input can comprise minimal-contact user input, such as input that may not require contact but may nevertheless result in contact which can be minimal, unsubstantial, unintended, or inconsequential. Reducing physical contact may improve sanitation, improve speed and efficiency of transferring physiological monitoring, and reduce complexities of transferring physiological monitoring such as by reducing the number of steps needed to transfer physiological monitoring. In some implementations, the one or more hardware processors may establish wireless communication at block 547 based on a proximity of a destination monitoring hub to one or more sensors in combination with a user input.

The one or more hardware processors can establish a wireless communication at block 547 between a destination monitoring hub and a plurality of sensors. The one or more hardware processors can establish wireless communication with a plurality of sensors at a single time. The one or more hardware processors can establish wireless communication with a plurality of sensors in response to a single user input. The one or more hardware processors can establish wireless communication with a plurality of sensors in response to a single request to transfer physiological monitoring.

At block 549, the one or more hardware processors can receive real-time physiological data at the destination monitoring hub originating from one or more sensors. The one or more hardware processors can receive the real-time physiological data via the wireless communication established at block 547. The real-time physiological data can include data generated by the one or more sensors and transmitted to the one or more hardware processors in real-time. For example, the one or more hardware processors may receive the physiological data at a substantially same time as the one or more sensors generate the physiological data. As another example, the one or more hardware processors may receive the physiological data with a minimal time delay after the one or more sensors generate the physiological data which time delay may be imperceptible to humans. The one or more hardware processors may receive the real-time physiological data continuously. For example, the real-time physiological data may include a continuous stream of data. The one or more hardware processors may receive the real-time physiological data periodically. For example, the real-time physiological data may include data generated periodically by the one or more sensors.

At block 551, the one or more hardware processors can optionally communicate real-time physiological data to a server. The server can be associated with a healthcare facility. The real-time data can be provided to the server when the user is within a healthcare facility such as from a destination monitoring hub. The real-time data can be provided to the server when a user is not within a healthcare facility such as from a user device when a user it a home which can facilitate remote healthcare. In some implementations, the server can process real-time physiological data originating from the sensors such as to generate physiological parameters, notifications, user interface data, etc. The server can process the real-time physiological data with historical physiological data which can result in more accurate processing.

At block 552, the one or more hardware processors can optionally process the real-time physiological data originating from the sensors such as to generate physiological parameters, notifications, user interface data, etc. The one or more hardware processors can process the real-time physiological data with historical physiological data which can result in more accurate processing. In some implementations, the one or more hardware processors may process the real-time physiological data only if the server does not process the data

At block 553, the one or more hardware processors can cause display of indicia of physiological data which can include real-time physiological data and/or historical physiological data. In some implementations, the one or more hardware processors can generate user interface data to cause display of the indicia of the physiological data. The one or more hardware processors can render the display via the destination monitoring hub. In some implementations, the one or more hardware processors can transmit user interface data to a remote computing device, such as smartwatch, smartphone, tablet, PC, wearable device, monitoring device which can render the display. Advantageously, the one or more hardware processors may have access to both real-time physiological data as well as historical physiological data (which may have been generated by the one or more sensors prior to establishing wireless communication with the one or more sensors) which may improve physiological monitoring such as by reducing data loss and providing a more comprehensive view of physiological data of a subject. The one or more hardware processors can cause display of indicia of real-time physiological data in combination with historical physiological data with minimal or no breaks or discontinuities appearing in the display of the physiological data. The one or more hardware processors can cause display of indicia of physiological data as if the one or more hardware processors had been receiving physiological data from the sensor(s) at a time prior to establishing wireless communication at block 547.

In some implementations, at block 553, the one or more hardware processors may not generate user interface data corresponding to the historical physiological data. For example, at block 544, the one or more hardware processors may receive user interface data corresponding to at least the historical physiological data. User interface data received at block 544 may have been generated by an origin monitoring hub which may be similar or of a same type as a destination monitoring hub and/or generated by a server. Accordingly, user interface data generated by an origin monitoring hub and/or by a server and received at a destination monitoring hub may be compatible with the destination monitoring hub such that the destination monitoring hub may not need to re-generate user interface data for rendering a display corresponding to the historical physiological data. Advantageously, eliminating the need to generate redundant user interface data can reduce processing requirements, improve processing speed and efficiency, and reduce the time needed to render a display including indicia of physiological data which may improve physiological monitoring and healthcare provided to a subject. In some implementations, an origin monitoring hub and a destination monitoring hub may be different types and/or comprise non-similar displays such that user interface data generated at an origin monitoring hub may not be compatible with a destination monitoring hub. In such implementations, a destination monitoring hub can generate user interface data corresponding to historical physiological data as described at block 553.

FIG. 5B is a flowchart illustrating an example method 500F of monitoring a user as the user transitions between environments. This process, in full or parts, can be executed by one or more hardware processors, whether they are associated with a singular or multiple computing devices, and even devices in remote or wireless communication. By way of example, the one or more hardware processors executing method 500F can be associated with any of the example servers shown and/or described herein, such as a server associated with a healthcare monitoring environment. The implementations of this method may vary and can involve modifications like omitting blocks, adding blocks, and/or rearranging the order of execution of the blocks. Method 500F serves as an example and is not intended to restrict the present disclosure.

At block 581 a computing device such as a server (for example, one or more hardware processors of a computing device executing program instructions) can receive a request to establish physiological monitoring of a patient with a monitoring hub in a healthcare environment. The monitoring hub can be an in-room display terminal device. The monitoring hub can be a portable monitoring hub. The healthcare environment can be a hospital, clinic, ambulance, etc. The request can be received via the monitoring hub. For example, the monitoring hub can generate the request responsive to NFC at the monitoring hub with another device, such as a user ID badge, a user ID bracelet, a user phone, a user watch, a home monitoring device, etc. A request via the monitoring hub can include other types of user input such as a button press, voice commands, etc. In some implementations, the request can originate from user interaction with a user device, such as a watch, phone, or home monitoring device. In some implementations, the request can include an authorization to establish physiological monitoring and/or to access historical physiological data (for example, discussed at block 583). In some implementations, generating the request via NFC at a monitoring can serve as authorization to access historical physiological data.

At block 582 the computing device can access real-time physiological data of the patient originating from a physiological monitoring device in the healthcare environment. The physiological monitoring device can be a physiological sensor, such as any of the example sensors shown and/or described herein.

At block 583 the computing device can access historical physiological data of the patient originating from a home monitoring device. The home monitoring device can include a physiological sensor. The home monitoring device be a wearable device such as a watch. The home monitoring device can be a portable device such as a phone. The home monitoring device can be in a fixed location, such as integrated within a home. The home monitoring device can be a soundbar with integrated sensors, a scale, etc. The home monitoring device can generate physiological data of the patient when the user is at home and can also generate physiological data of the patient when the user is at other locations remote to their home. The phrase “home monitoring device” is not intended to limit the scope such as by restricting the home monitoring device to use within a home. The historical physiological data can include data that was generated by the home monitoring device before the patient is admitted to a healthcare environment. The historical physiological data can include data that was generated over a time frame spanning minutes, hours, days, weeks, etc. The historical physiological data can originate from more than one device. The computing device can access the historical physiological data from a server. For example, a server associated with a healthcare environment can retrieve the historical physiological data from another server (for example, a proprietary server that maintains a user's physiological data). In some implementations, the computing device can access the historical physiological data directly from a user device. For example, the computing device (for example, a server and/or monitoring hub) can retrieve the historical physiological data from a user's watch, phone, etc. For example, responsive to NFC communication between a patient's watch and a monitoring hub in a healthcare environment, the monitoring hub can access historical physiological data stored on the patient's watch communicated from the watch to the monitoring hub.

The amount of historical physiological data that the computing device accesses and/or retrieves can depend on the historical physiological data itself, or aspects thereof, such as the type of data, physiological conditions identified in the data, the type of device or sensor from the which the data originated, etc. For example, the computing device can access and/or retrieve a subset of the physiological data corresponding to a certain time frame if the physiological data indicates that certain health conditions (such as apnea, afib, hypoxia, etc.) were occurring during that time frame.

In some implementations, the computing device can parse the historical physiological data. Parsing the data can include formatting the data into a structured format. Parsing the data can include reformatting the data to correspond to a format of the real-time sensor data. The computing device can parse the data based on a sensor or device from which the data originates.

In some implementations, the computing device can generate a summary of the historical physiological data. The summary can be accessed and reviewed by a healthcare provider. The summary can include indications of relevant physiological parameters, whether physiological parameters have surpassed relevant thresholds, whether physiological events have likely occurred, alerts that have been generated, etc. In some implementations, the computing device can generate the summary with artificial intelligence. For example, the computing device can implement machine learning on a database of information (for example, other patient information) to determine relevant information to include in the summary. For example, the computing device can include an indication in the summary if the historical physiological data deviates from a data in a database of relevant information.

At decision block 584 the computing device can determine whether the home monitoring device from which the historical physiological data originates is an approved device. Approved devices can include devices that are medically approved, such as approved by the Food and Drug Administration (FDA). Approved devices can include devices that are approved for use within a certain environment (for example, within a hospital, operating room, etc.) or approved for a particular use, or approved to be used by a particular user. For example, some wearable devices may be approved (for example, medically approved by the FDA) while other wearable devices may not be approved. Such approval may depend on the manufacturer/distributor of such devices. Data originating from an approved device may be more accurate and/or reliable than data originating from an unapproved device. Thus, determining whether data originates from an unapproved device can improve more accurately monitoring the patient.

The computing device can determine whether data originates from an approved device based on metadata of the data. For example, metadata of the historical physiological data can indicate a device or sensor from which the data originates. The computing device can then determine if the device is approved. In some implementations, the computing device can determine whether data originates from an approved device based on a server from which the data is retrieved. For example, the computing device can retrieve the data from a server that only stores data from approved devices. Thus, the computing device can know that all data accessed from such a server would originate from an approved device.

If the computing device determines that the historical physiological data does not originate from an approved device, the method can proceed to block 585. If the computing device determines that the historical physiological data originates from an approved device, the method can proceed to block 586.

At decision block 585 the computing device can determine whether to use data from an unapproved device. A user may be able to adjust settings to enable or disable using data from an unapproved device. If the computing device determines to not use data from an unapproved device, the method can proceed to block 583. If the computing device determines to use data form an unapproved device the method can proceed to block 586.

At block 586 the computing device can optionally indicate whether the historical physiological data originates from an approved device or an unapproved device. For example, the computing device can mark and/or update metadata of the historical physiological data to indicate the device (for example, approved, unapproved) from which the data originates.

At block 587 the computing device can process the real-time physiological data originating from the monitoring device in the healthcare environment in combination with the historical physiological data originating from the home monitoring device. Processing the physiological data can include generating physiological parameters, generating thresholds, generating alarms, etc. The historical physiological data can serve as context for processing the real-time physiological data. For example, the computing device can analyze the real-time data in combination with the historical data to determine physiological parameters. As another example, the computing device can calculate a threshold as a moving average from real-time data and historical data. Thus, accessing the historical physiological data can provide additional data with which to improve accuracy of processing the real-time physiological data. In some implementations, the computing device can weight the physiological data based on whether it originated from an approved device. For example, the computing device can assign a smaller weight to data originating from an unapproved device to reduce the effects of potentially unreliable data in a computation. In some implementations, the computing device can assign a confidence score to the physiological parameters based on whether the physiological data used to determine the parameters originated from an approved device or an unapproved device.

At block 588 the computing device can method the real-time physiological data without the historical physiological data. For example, in some implementations, such as when historical physiological data originates from an unapproved device, processing the historical physiological data may introduce too much risk for unreliable data. Thus, the computing device may advantageously ignore historical physiological data when processing real-time physiological data which may improve accuracy. In some implementations, the computing device can indicate that the historical physiological data originated from an unapproved device, such as my marking and/or updating metadata of the historical physiological data, even if the historical physiological data is ignored from further processing.

At block 589 the computing device can optionally generate user interface data for rendering indicia of the real-time physiological data and the historical physiological data and/or can cause a monitoring hub to display said indicia. The indica can include values of physiological parameters, trend lines, graphs, animations, etc. The indica can be displayed at the monitoring hub in the healthcare environment. Advantageously, indicia of the real-time data can be displayed in combination with indicia of the historical physiological data which may improve physiological monitoring of the patient. For example, a trend line can include a greater amount of data extending back further in time by virtue of having access to the historical physiological data. In some implementations, the indicia can include an indication of whether the physiological data originated from an approved device or an unapproved device. Thus, a person viewing the indicia on display can easily ascertain the source (or the reliability) of the physiological data.

FIG. 5C is a flowchart illustrating an example method 500G of monitoring a user as the user transitions between environments. This process, in full or parts, can be executed by one or more hardware processors, whether they are associated with a singular or multiple computing devices, and even devices in remote or wireless communication. By way of example, the one or more hardware processors executing method 500G can be associated with any of the example user devices shown and/or described herein, such as a watch or phone. The implementations of this method may vary and can involve modifications like omitting blocks, adding blocks, and/or rearranging the order of execution of the blocks. Method 500G serves as an example and is not intended to restrict the present disclosure.

At block 591 a computing device such as a user device (for example, one or more hardware processors of a computing device executing program instructions) can receive a request to access historical physiological data of a user. For example, a remote computing device, such as a server, can communicate a request to a user device to access physiological data stored on the user device. In some implementations, a healthcare provider may desire to access historical physiological data of the user, such as data that was generated by a home monitoring device before a user is admitted to a healthcare environment. For example, a first responder may generate a request to retrieve the user's historical physiological data which can aid the first responder in determining how to administer health care to the user. For example, a first responder may be responding to an adverse health condition suffered by the user and historical physiological data (for example, generated by a watch or other home monitoring device) may include information relevant to the adverse health condition. As another example, when a patient is admitted to a hospital, historical physiological data may provide information to a healthcare provider on the patient's medical history.

At decision block 592 the computing device can determine whether a health event has been detected. A health event can include an adverse physiological condition of the user, such as cardiac arrythmia, cardiac arrest, unconsciousness, reduced blood oxygen concentration, opioid overdose, carbon monoxide poisoning, etc. A health event can indicate the user is incapacitated or at reduced capacity to function. The computing device can determine whether a health event has occurred from physiological data. For example, the computing device can access physiological data stored on the computing device (for example, physiological data stored on a watch generated by sensors integrated with the watch) to determine whether the health event has occurred.

In response to determining that the health event has occurred, the method can proceed to block 593. In response to determining that the health event has occurred, the method can proceed to decision block 594.

At block 593 the computing device can reduce an authorization requirement to access the data. For example, if a health event has been detected indicating a user does not have capacity to provide authorization (for example, the user is unconscious), the computing device can reduce or bypass an authorization layer required before communicating the physiological data. For example, in some cases the authorization required from a user can be set such that no authorization is required.

At decision block 594 the computing device can determine whether a user authorization satisfies the authorization requirement to access the historical physiological data as requested. The authorization requirement can be dynamically adjusted (for example, at block 593) and thus the authorization check at block 594 may vary depending on the situation. Advantageously, a user can retain control and privacy over their physiological data to prevent third parties from accessing such data without the user's authorization.

At block 595 the computing device can provide access to the historical physiological data. Providing access to the historical physiological data can include communicating the historical physiological data to a remote computing device directly from the user device. Providing access to the historical physiological data can include causing the historical physiological data to be communicated to a remote computing device from a server. Providing access to the historical physiological data can include displaying physiological data on a user device for a person to view the data.

### Example Wearable Systems

A wearable system (which can also be referred to herein as a “wearable sensor system” or “wearable physiological sensor system”) can include an electronic device and a wearable device. The electronic device can measure one or more physiological parameters of the subject. For this, the electronic device can include one or more sensors as described herein. The wearable device can operably position the electronic device. The wearable device can include a main body (which can also be referred to as a “body portion”) and a securement portion. The main body of the wearable device can have a cavity for positioning the electronic device, and the securement portion of the wearable device can be connected to the main body and secure the main body to the subject. In some implementations, the electronic device mechanically and/or electrically connects with the wearable device when the electronic device and the wearable device are secured to one another. In some implementations, the electronic device and the wearable device are formed as a unitary system.

Implementations of the wearable systems described herein can be secured to various portions of the subject's body including a wrist, lower arm, upper arm, and/or upper body in a variety of manners and/or using a variety of methods. Alternatively, or in addition, implementations of the wearable systems described herein can be secured and/or placed adjacent to and/or around portions of a body of the subject other than the wrist, lower arm, upper arm, and/or upper body such as adjacent to and/or around an ankle, a lower leg, an upper leg, a lower body, and/or a torso or mid portion of the subject's body among other regions or portions of the subject's body. Accordingly, while implementations of the wearable systems disclosed herein may be illustrated and/or described with reference to a wrist of the subject, such description is not intended to be limiting, and the various implementations of the wearable systems disclosed herein can be utilized in connection with other portions of the subject's body.

The wearable systems described herein (for example, wearable system 3000) and/or aspects thereof, such as the electronic devices described herein (for example, electronic devices 2700, 2700′. 3200) and/or the wearable devices described herein (for example, wearable devices 3100) can be similar or identical to and/or incorporate any of the features described and/or illustrated with respect to any of the devices, assemblies, and/or systems described and/or illustrated in U.S. Patent Pub. No. 2024/0081656, which is hereby incorporated by reference in its entirety and made a part of this application. Any of the wearable systems, wearable devices, and/or electronic devices described and/or illustrated in U.S. Patent Pub. No. 2024/0081656 incorporated by reference herein can incorporate any of the features described and/or illustrated with respect to any of the devices, assemblies, and/or systems described and/or illustrated herein and are made a part of this application.

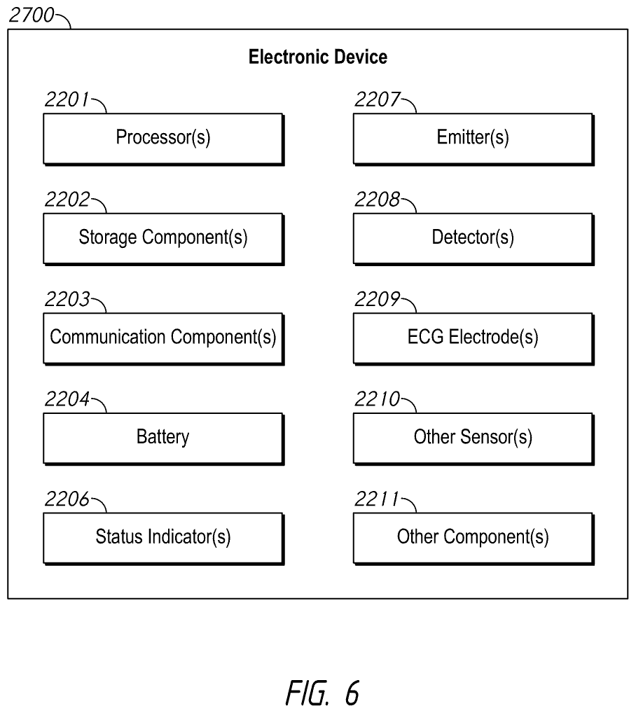

FIG. 6 illustrates a schematic diagram of certain features which can be incorporated in any of the implementations of the wearable systems and/or associated electronic devices described herein. As shown, an electronic device 2700 can include one or more emitters 2207 and one or more detectors 2208. Such one or more emitters 2207 and one or more detectors 2208 can form a pulse oximetry sensor of the electronic device 2700. Also shown, the electronic device 2700 can include one or more processors 2201, one or more storage components 2202 (which can also be referred to as “storage devices” or “memory”), one or more communication components 2203 (which can also be referred to as “communication module(s)”), a battery 2204, one or more status indicators 2206, one or more ECG electrodes 2209, one or more other sensors 2210, and/or one or more other components 2211. While certain features of the electronic device 2700 are shown in FIG. 6 that can be incorporated therein, any of such features are optional. Furthermore, the electronic device 2700 can include other features than shown in FIG. 6.

The one or more emitters 2207 and the one or more detectors 2208 of the electronic device 2700 can be utilized to obtain physiological information indicative of one or more physiological parameters of the subject. These parameters can include various blood analytes such as oxygen, carbon monoxide, methemoglobin, total hemoglobin, glucose, proteins, glucose, lipids, a percentage thereof (for example, concentration or saturation), and the like. The one or more emitters 2207 and the one or more detectors 2208 can also be used to obtain a photoplethysmograph, a measure of plethysmograph variability, pulse rate, a measure of blood perfusion, and the like. Information such as oxygen saturation (SpO2), pulse rate, a plethysmograph waveform, respiratory effort index (REI), acoustic respiration rate (RRa), EEG, ECG, pulse arrival time (PAT), perfusion index (PI), pleth variability index (PVI), methemoglobin (MetHb), carboxyhemoglobin (CoHb), total hemoglobin (tHb), and/or glucose, can be obtained from the electronic device 2700 (for example, by a wearable system incorporating electronic device 2700) and data related to such information can be processed and/or transmitted by the electronic device 2700 (for example, via communication component(s) 2203 of the electronic device 2700) to a separate device (for example a separate computing device such as a mobile phone or monitoring hub). The one or more emitters 2207 and the one or more detectors 2208 can be optically based and, for example, utilize optical radiation. Further, the one or more emitters 2207 can serve as a source of optical radiation that can be directed towards tissue (which can also be referred to as a “tissue site”) of the subject when a wearable system incorporating electronic device 2700 is in use. The electronic device 2700 can include one, two, three, four, five, six, seven, or eight or more emitters 2207 and/or one, two, three, four, five, six, seven, or eight or more detectors 2208. The one or more emitters 2207 can be one or more light-emitting diodes (LEDs) (for example, such as low-power, high-brightness LEDs), laser diodes, incandescent bulbs with appropriate frequency-selective filters, and/or any other source(s) of optical radiation and/or any combinations of the same, or the like. The one or more emitters 2207 can emit optical radiation of one or more wavelengths and can emit visible and near-infrared optical radiation. The one or more detectors 2208 can be configured to detect optical radiation generated by the one or more emitters 2208. The one or more detectors 2208 can detect optical radiation that attenuates through and/or is reflected by tissue of the subject, for example, tissue of the subject's wrist, lower arm, and/or upper arm. The one or more detectors 2208 can output one or more signals responsive to the detected optical radiation. In some implementations, the one or more detectors 2208 can be one or more photodiodes, phototransistors, or the like. Any one or more of the subject's physiological measurements made via the one or more emitters 2207 and the one or more detectors 2208 can be transmitted to a separate device in communication with the electronic device 2700 for display.

The one or more processors 2201 (which can also be referred to herein as “hardware processors” or “computer hardware processors”) can be configured, among other things, to process data, execute instructions to perform one or more functions, and/or control the operation of the electronic device 2700. For example, the one or more processors 2201 can control operation of the one or more emitters 2207, the one or more detectors 2208, the one or more other sensors 2210, and/or the one or more other components 2211 of the electronic device 2700. As another example, the one or more processors 2201 can process signals and/or physiological data received and/or obtained from the one or more detectors 2208, the one or more other sensors 2210, and/or the one or more other components 2211 of the electronic device 2700. Further, the one or more processors 2201 can execute instructions to perform functions related to storing and/or transmitting such signals and/or physiological data received and/or obtained from the one or more detectors 2208, the one or more other sensors 2210, and/or the one or more other components 2211 of the electronic device 2700. The processor 2201 can execute instructions to perform functions related to storing and/or transmitting any or all of such received data.

The one or more storage component(s) 2202 (which can also be referred to herein as “storage device(s)”) can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. Such stored data can be processed and/or unprocessed physiological data obtained from by the electronic device 2700, for example. In some implementations, the one or more storage component(s) 2202 can include a memory storage element that stores, in non-volatile memory, information used to help maintain a standard of quality associated with the electronic device 2700. Illustratively, such memory can store information regarding whether the electronic device 2700 has been previously activated and whether the electronic device 2700 has been previously operational for a prolonged period of time, such as, for example, four hours, one day, two days, five days, ten days, twenty days, a month, multiple months, or any period of time. The information stored in such memory can be used to help detect improper re-use of the electronic device 2700, for example.

The communication component(s) 2203 (which can also be referred to herein as a “communication module”) can facilitate communication (via wires and/or wireless connection) between the electronic device 2700 and separate devices, such as separate monitoring, computing, electrical, and/or mobile devices. For example, the communication component(s) 2203 can be configured to allow the electronic device 2700 to wirelessly communicate with other devices, systems, and/or networks over any of a variety of communication protocols. The communication component(s) 2203 can be configured to use any of a variety of wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like or as described herein. The communication component(s) 2203 can allow data and/or instructions to be transmitted and/or received to and/or from the electronic device 2700 and separate computing devices. The communication component(s) 2203 can be configured to transmit (for example, wirelessly) processed and/or unprocessed physiological parameters, data and/or other information to one or more separate computing devices, which can include, among others, a patient monitor, a mobile device (for example, an iOS or Android enabled smartphone, tablet, laptop), a desktop computer, a server or other computing or processing device for display and/or further processing, among other things. Such separate computing devices can be configured to store and/or further process the received physiological parameters, data, and/or other information, to display information indicative of or derived from the received parameters, data, and/or information, and/or to transmit information—including displays, alarms, alerts, and notifications—to various other types of computing devices and/or systems that can be associated with the subject, a hospital, a caregiver (for example, a primary care provider), and/or a user (for example, an employer, a school, friends, family) that have permission to access the subject's data. As another example, the communication component(s) 2203 of the electronic device 2700 can be configured to wirelessly transmit processed and/or unprocessed obtained physiological parameters, data, information and/or other information (for example, motion and/or location data) to a mobile phone or monitoring hub which can include one or more hardware processors configured to execute an application that generates a graphical user interface displaying information representative of the processed or unprocessed physiological parameters, data, information and/or other information obtained from the electronic device 2700. The communication component(s) 2203 can be and/or include a wireless transceiver. The communication component(s) 2203 can include one or more antenna.

The battery 2204 can provide power for hardware components of the electronic device 2700 described herein. The battery 2204 can be rechargeable. For example, the battery 2204 can be a lithium, a lithium polymer, a lithium-ion, a lithium-ion polymer, a lead-acid, a nickel-cadmium, or a nickel-metal hydride battery. In some implementations, the battery 2204 can be charged/recharged by wirelessly charging (for example, via a wireless charging pad), by solar energy (for example, via a solar collector if incorporated in the electronic device 2700), and/or by kinetic motion (for example, via an internal mechanism if incorporated that can convert kinetic motion into electrical power). In some cases, the battery 2204 can be removed, or the battery 2204 can be integrated within and/or a permanent part of the electronic device 2700. In some implementations, the battery 2204 can be non-rechargeable. Additionally or alternatively, the electronic device 2700 can be configured to obtain power from a power source that is external to the electronic device 2700. For example, the electronic device 2700 can include or can be configured to connect to a cable which can itself connect to an external power source to provide power to the electronic device 2700.

The one or more status indicators 2206 can be configured to provide and/or indicate a status of the electronic device 2700 and/or a status of one or more physiological parameters of the subject determined by the electronic device 2700 and/or any devices in communication with the electronic device 2700. In some implementations, the one or more status indicators 2206 can be configured to indicate a status of the electronic device 2700, such as whether the electronic device 2700 is in an operational (“on”) mode, whether the electronic device 2700 is pairing or has paired with a separate device, whether an error has been detected, and/or a power level of the electronic device 2700 (for example, a charge of battery 2204). For example, the one or more status indicators 2206 can be configured to light up and/or cast optical radiation of one or more wavelengths from one or more portions of the electronic device 2700. As another example, the one or more status indicators 2206 can be configured to light up and/or emit optical radiation from one or more portions of the electronic device 2700. The one or more processors 2201 can be in communication with the one or more status indicators 2206 and can be configured to instruct the one or more status indicators 2206 to cause any of such above-described status indications and/or lighting. In some cases, the one or more status indicators 2206 can be configured to provide optical radiation (for example, light) feedback to the subject when the electronic device 2700 is secured to the subject and/or when electronic device 2700 and a wearable device are connected together. In some implementations, electronic device 2700 can be configured to cause optical radiation feedback to the subject (when the electronic device 2700 is secured to the subject by a wearable device) responsive to one or more physiological parameters determined by electronic device 2700 and/or by any devices (such as separate computing and/or mobile devices, for example, a mobile phone) in communication with the electronic device 2700. The one or more processors 2201 can instruct the one or more status indicators 2206 to emit or stop emitting optical radiation and/or instruct the one or more status indicators 2206 to alter a characteristic of optical radiation (for example, increase/reduce optical radiation brightness, change optical radiation wavelength and/or color, change a rate of blinking of optical radiation, etc.) responsive to the one or more determined physiological parameters. Such action by the one or more processors 2201 can dynamically track with physiological parameter determination over time, for example. As an example, in some implementations, the one or more processors 2201 can provide instructions to the one or more status indicators 2206 (such as those discussed above) responsive to a condition of the subject using the electronic device 2700. For example, if one or more physiological parameters determined by the electronic device 2700 and/or any devices in communication with the electronic device 2700 are indicative of hypoxemia (low blood oxygen) when the subject is using the electronic device 2700, the one or more processors 2201 can instruct the one or more status indicators 2206 to produce optical radiation to notify the subject and/or their care providers to restore proper breathing and/or safe blood oxygen levels. As another example, if one or more physiological parameters determined by the electronic device 2700 and/or any devices in communication with the electronic device 2700 are indicative of edema (swelling caused by excess fluid trapped in body tissue) when the subject is using the electronic device 2700, the one or more processors 2201 can instruct the one or more status indicators 2206 to cause optical radiation to be emitted from the electronic device 2700 as described above. In some implementations, the one or more processors 2201 and/or any devices in communication with the electronic device 2700 can instruct the one or more status indicators 2206 to cause optical radiation to be emitted if a determined subject physiological parameter of interest meets and/or exceeds a set threshold, meets and/or falls below a set threshold, and/or meets, exceeds, and/or falls below a set range. In some cases, optical radiation emitted from the one or more status indicators 2206 can correspond to an alert, an alarm, a notification, and/or any other situation wherein the subject and/or a care provider need to intervene in the subject's care. The one or more status indicators 2206 can be positioned within various portions of the electronic device 2700 such that optical radiation emitted from the one or more status indicators emit out of and/or through one or more holes and/or one or more openings in the electronic device 2700, such as shown herein.

The one or more ECG electrodes 2209 of the electronic device 2700, when included, can be utilized to obtain physiological information indicative of one or more physiological parameters of the subject. For example, the one or more ECG electrodes 2209 can be configured to contact the subject (for example, contact the subject's skin) and output one or more signals responsive to the subject's cardiac electrical activity. The one or more processors 2201 can be configured to receive the one or more signals from the ECG electrodes 2209 responsive to the subject's cardiac electrical activity and determine an ECG of the subject responsive to such one or more signals (for example, automatically determine an ECG of the subject). The electronic device 2700 can include one, two, three, four, five, six, seven, eight, nine, ten, or more ECG electrodes 2209. The one or more ECG electrodes 2209 can include one or more negative electrodes, one or more positive electrodes, and one or more reference electrodes. Such negative electrode(s), positive electrode(s), and reference electrode(s) can be electrically isolated from one another. The one or more ECG electrodes 2209 can form an ECG sensor of the electronic device 2700. In some implementations, the electronic device 2700 further includes an ECG amplifier configured to receive analog signals from the ECG electrodes 2209, which can output amplified analog signals to an analog-digital converter that can also be included in the electronic device 2700. The amplified analog signals can include an ECG differential between the positive and negative electrodes. The analog-digital converter can output a digital signal based on the analog signals from the one or more ECG electrodes 2209 to the one or more processors 2201 of the electronic device 2700 for determination of the subject's ECG. In some implementations, the one or more ECG electrodes 2209 can optionally make physiological measurements based on the obtained ECG, for example, a heart rate, a respiratory rate, and/or otherwise of the subject. The subject's ECG waveform and/or the other physiological measurements made from the one or more ECG electrodes 2209 can be transmitted to a separate device in communication with the electronic device 2700 for display.

In some implementations, the electronic device 2700 can include one or more other sensor(s) 2210. The other sensor(s) 2210 can include one or more of a temperature sensor, a blood pressure monitor, an acoustic sensor (for example, an audio transducer), a location sensor (for example, a GPS sensor), and/or any sensor configured to obtain physiological information indicative of one or more physiological parameters of the subject and/or other information of the subject (for example, a number of steps taken or a distance traveled by the subject). Additionally, or alternatively, such other sensor(s) 2210 can comprise an inertial sensor (which can also be referred to herein as a “motion sensor”), for example, including one or more accelerometers and/or gyroscopes, that can be utilized to determine motion of the subject and/or a portion of the subject's body (for example, wrist, lower arm, upper arm, upper body). In some implementations where the electronic device 2700 includes an inertial sensor, the processor(s) 2201 can determine whether the portion of the subject's body that the electronic device 2700 is secured to is moving. Furthermore, in some implementations, the processor(s) 2201 can determine the type of movement being performed and/or an exertion level of the subject. Such determination can be used for a determination of a caloric expenditure, for example. The other sensor(s) 2210 can be disposed on, within, and/or be operably positioned by the electronic device 2700 and/or a wearable device configured to secure the electronic device 2700. The other sensor(s) 2210 can be operably connected to the one or more processors 2201, which can control operation of the other sensor(s) 2210 and/or process data received from the other sensor(s) 2210.

In some implementations, the electronic device 2700 can include one or more other component(s) 2211. The other component(s) 2211 can include one or more of an audio component configured to produce sound (for example, a buzzer or a speaker), a vibration motor configured to vibrate one or more portions of the electronic device 2700, or any other component. In some implementations, the one or more other components 2211 can be configured to produce an output to the subject. For example, the electronic device 2700 can include a vibration motor that can be configured to vibrate one or more portions of the electronic device 2700, which in turn can vibrate one or more portions of the subject's body when the electronic device 2700 is secured to the subject. The one or more processors 2201 can be in communication with vibration motor and can be configured to instruct the vibration motor to cause any of such above-described vibration. In some cases, the vibration motor can be utilized to provide haptic feedback to the subject when the electronic device 2700 is secured to the subject. In some implementations, the electronic device 2700 can be configured to cause vibration of and/or provide haptic feedback to one or more portions of the subject's body (when the electronic device 2700 is secured to the subject) via the vibration motor responsive to one or more physiological parameters determined by electronic device 2700 and/or by any devices (such as separate computing, electrical, and/or mobile devices, for example, a patient monitor) in communication with the electronic device 2700. The one or more processors 2201 can instruct the vibration motor to cause vibration, cease vibrating, and/or instruct the vibration motor to alter a characteristic of vibration (for example, increase/reduce vibration rate, increase/reduce vibration strength, change vibration pattern, etc.) responsive to the one or more determined physiological parameters. Such action by the one or more processors 2201 can dynamically track with physiological parameter determination over time, for example. As an example, in some implementations, the one or more processors 2201 can provide instructions to the vibration motor (such as those discussed above) responsive to a condition of the subject using the electronic device 2700. For example, if one or more physiological parameters determined by the electronic device 2700 and/or any devices in communication with the electronic device 2700 are indicative of hypoxemia (low blood oxygen) when the subject is using the electronic device 2700 (for example, such as when sleeping), the one or more processors 2201 can instruct the vibration motor to vibrate to cause the subject to wake up in an attempt to restore proper breathing and/or safe blood oxygen levels. As another example, if one or more physiological parameters determined by the electronic device 2700 and/or any devices in communication with the electronic device 2700 are indicative of edema (swelling caused by excess fluid trapped in body tissue) when the subject is using the electronic device 2700, the one or more processors 2201 can instruct the vibration motor to cause vibration of a portion of the subject's body. In some implementations, the one or more processors 2201 and/or any devices in communication with the electronic device 2700 can instruct the vibration motor to cause a vibration if a determined subject physiological parameter of interest meets and/or exceeds a set threshold, meets and/or falls below a set threshold, and/or meets, exceeds, and/or falls below a set range. In some cases, a vibration of the vibration motor can correspond to an alert, an alarm, a notification, and/or any other situation wherein the subject and/or a care provider need to intervene in the subject's care. In some implementations, the one or more processors 2201 can instruct the vibration motor to vibrate responsive to a status of battery 2204 (for example, when a charge of the battery 2204 drops below a certain threshold). In some implementations, the one or more processors 2201 can instruct the vibration motor to vibrate responsive to the processors determining that a wearable device secured to the electronic device is an authorized product and/or an unauthorized product, responsive to a service life of the electronic device and/or the wearable device secured thereto being exceeded, and/or responsive to patient identification information being transferred to the electronic device (such as from a wearable device). In some implementations, electronic device 2700 can include more than one vibration motor, for example, two, or three or more vibration motors. Vibration motor(s) if included can be positioned within various portions of the electronic device 2700. The audio component, when included, can be configured to produce one or more sounds responsive to any one or more of the determinations described above with respect to the vibration motor(s). In some implementations, the audio component(s) and the vibration motor(s) can be operated simultaneously.

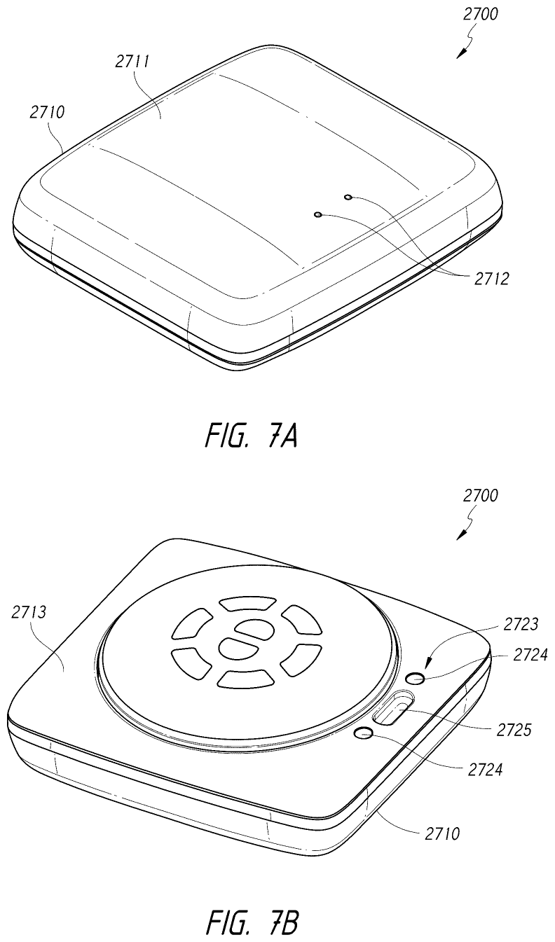

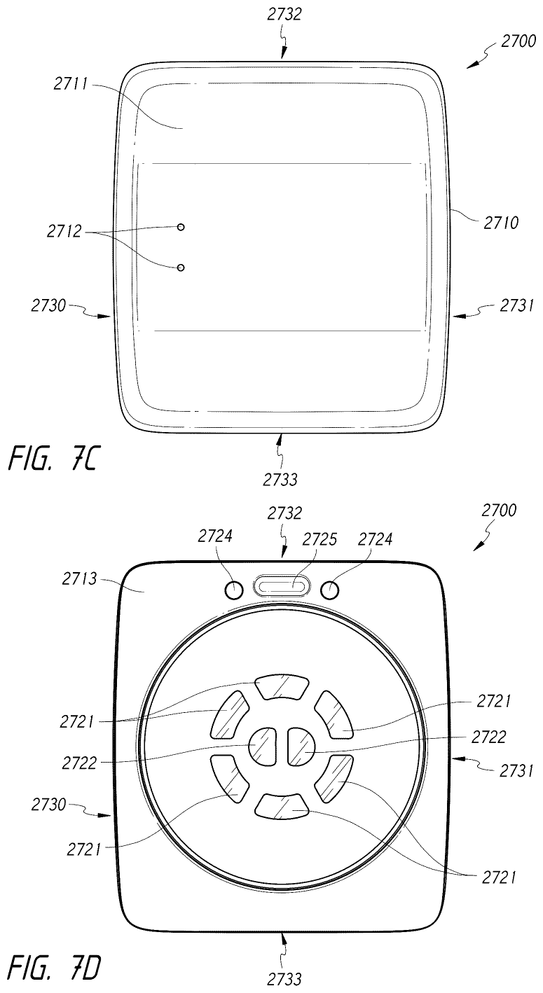

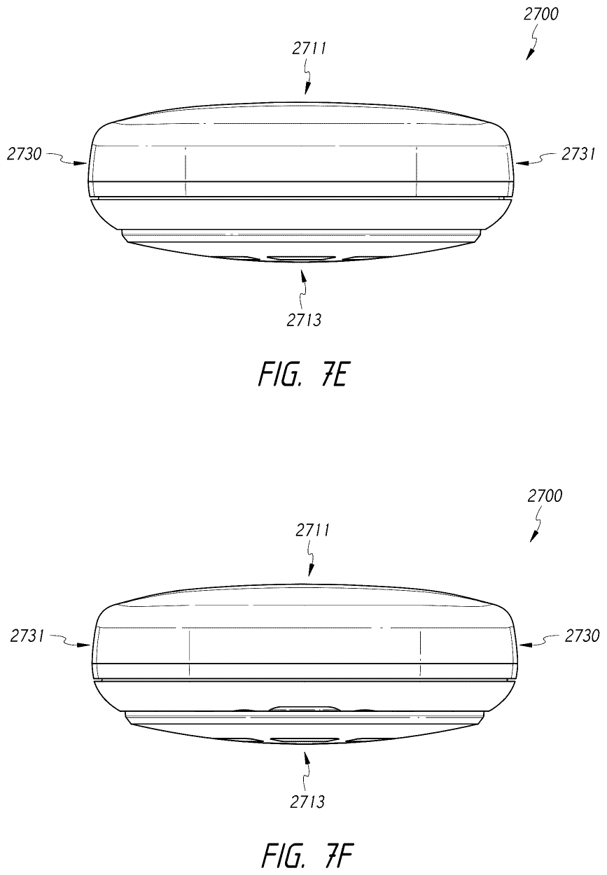

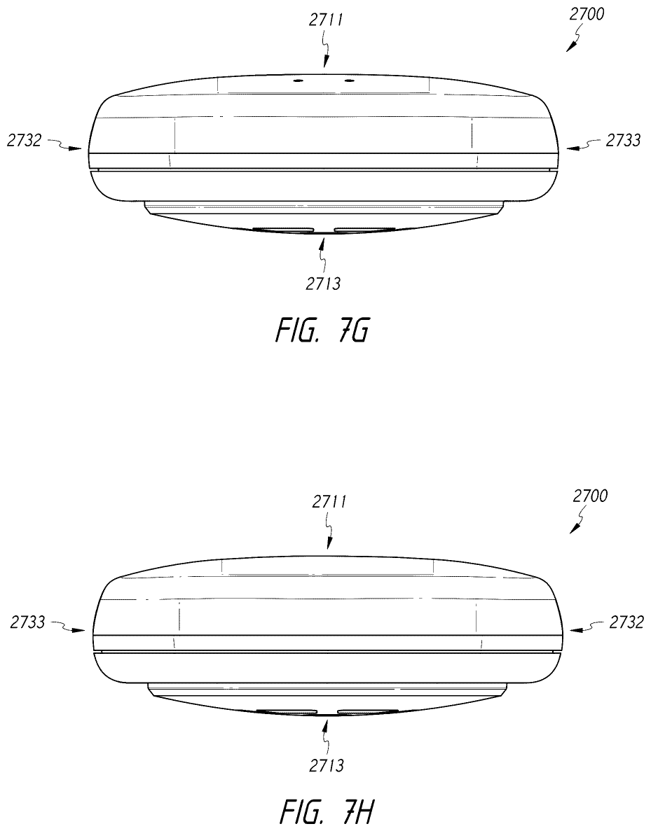

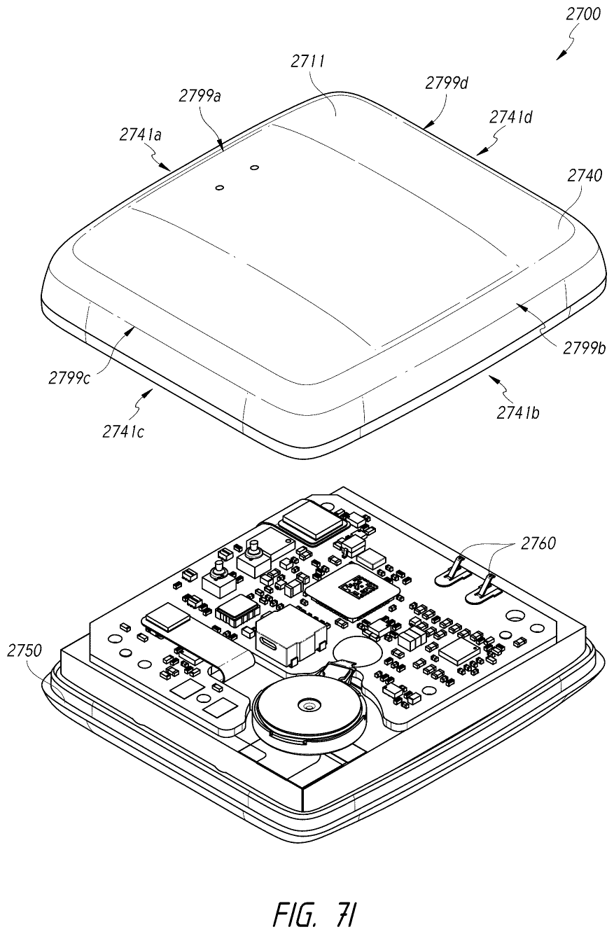

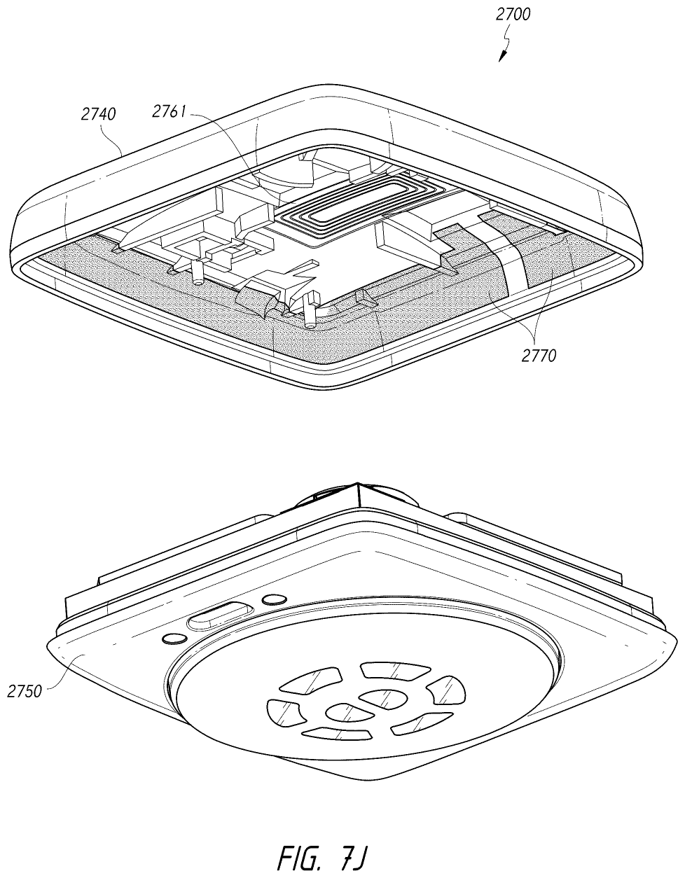

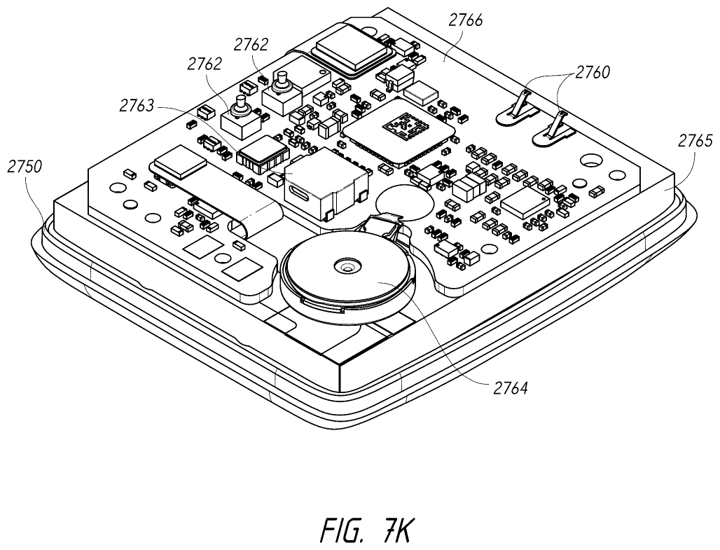

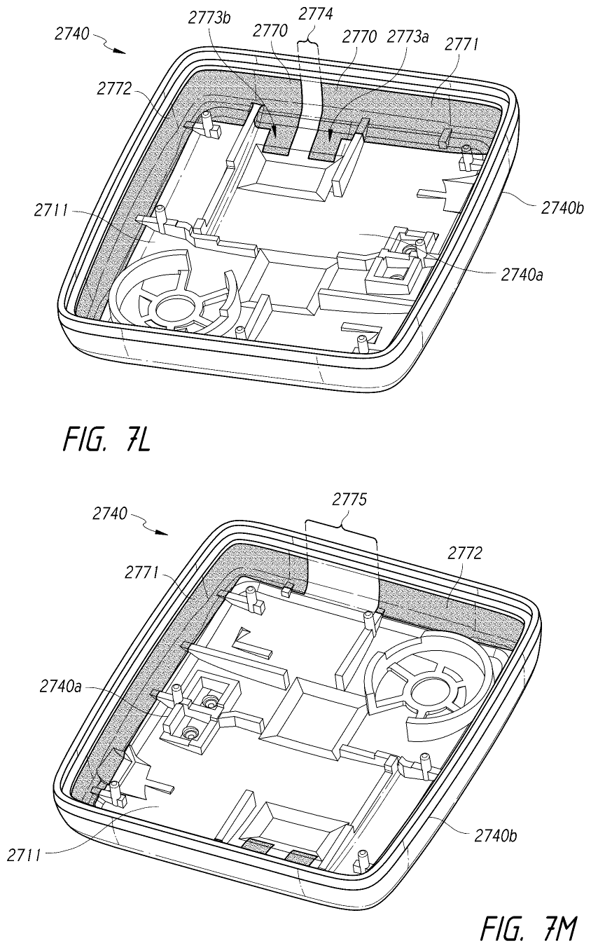

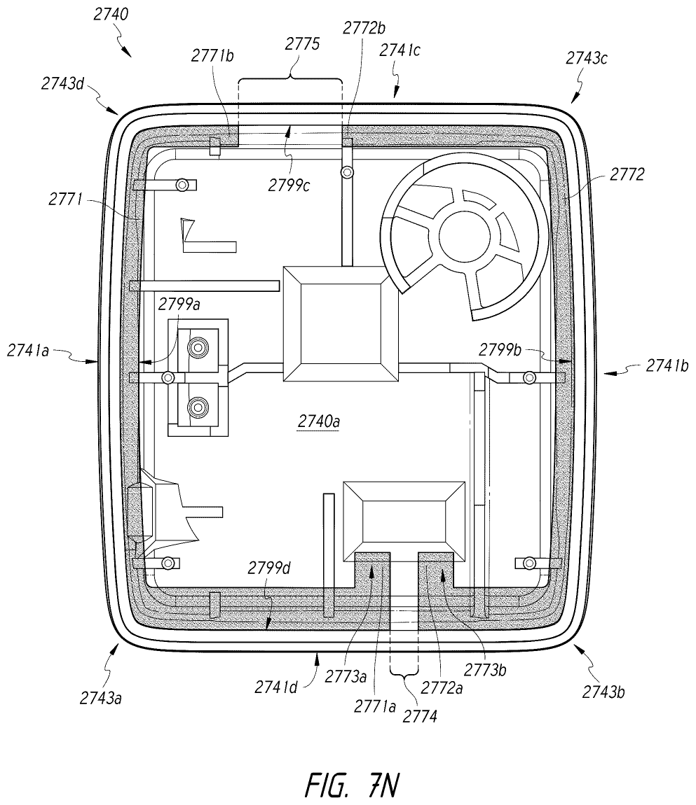

FIGS. 7A-7N illustrate various views of electronic device 2700. Electronic device 2700 can be incorporated into and/or can interact with (for example, removably secure to) any of the wearable devices described herein and/or with any of the wearable devices described in U.S. Patent Pub. No. 2024/0081656 incorporated by reference herein. Electronic device 2700 can interact in a similar or identical manner with any of the wearable devices described herein and/or in U.S. Patent Pub. No. 2024/0081656 incorporated by reference herein as described with respect to any of the other electronic devices disclosed herein and/or in U.S. Pub. No. 2024/0081656.

The electronic device 2700 can include any one or more of the features illustrated and/or discussed with respect to FIG. 6, for example, the processor(s) 2201, the storage component(s) 2202, the communication component(s) 2203, the battery 2204, the status indicator(s) 2206, the emitter(s) 2207, the detector(s) 2208, the ECG electrode(s) 2209, the other sensor(s) 2210, and/or the other component(s) 2211. Furthermore, the electronic device 2700 can be similar or identical to and/or incorporate any of the features described and/or illustrated with respect to any of the devices, assemblies, and/or systems described and/or illustrated in U.S. Patent Pub. No. 2024/0081656 incorporated by reference herein.

In some implementations, as shown in FIGS. 7A-7D, the electronic device 2700 has a generally rounded square shape. Alternatively, the electronic device 2700 can be circular, rectangular, oval (and/or elliptical), or another shape. The electronic device 2700 can include a housing 2710. Housing 2710 can include a top portion 2711 and a bottom portion 2713 (which can be opposite the top portion 2711). The bottom portion 2713 may face towards tissue of the subject when the electronic device 2700 is secured to the subject's body (for example, with a wearable device such as any of the wearable devices disclosed herein).

As shown in at least FIG. 7A, the electronic device 2700 can include status indicators 2712. The status indicators 2712 can provide and/or indicate a status of the electronic device 2700 and/or a status of one or more physiological parameters of the subject determined by the electronic device 2700 and/or any devices in communication with the electronic device 2700. The status indicators 2712 can be configured to operate according to any of the status indicators disclosed herein. The status indicators 2712 can be an implementation of status indicator(s) 2206 described herein. As shown in FIGS. 7B and 7D, the electronic device 2700 can have a charge port 2723 including electrical contacts 2724 and a feature 2725 (which may also be referred to as “charger engagement feature”) that can be similar or identical to the charge port 1312 including electrical contacts 1313 and the feature 1314 of the electronic device 1300 (and/or the charge port 1212 including electrical contacts 1213 and the feature 1214 of the electronic device 1200) described in U.S. Patent Pub. No. 2024/0081656 incorporated by reference herein. Electronic device 2700 can be configured to secure (for example, removably secure) to charger 1500 described in U.S. Patent Pub. No. 2024/0081656 incorporated by reference herein in a similar or identical manner as that described and/or illustrated with respect to electronic device 1200, 1300. Electrical contacts 2724 and feature 2725 can be configured to engage electrical contacts 1513 and feature 1514 of charger 1500 in a similar or identical manner as that described with respect to electrical contacts 1213 and feature 1214 of electronic device 1200 described in U.S. Patent Pub. No. 2024/0081656 incorporated by reference herein.

Electronic device 2700 can include one or more emitters for emitting light into tissue of a subject (when secured to a portion of the subject's body) and one or more detectors for detecting the emitted light after the attenuation through the tissue (for example, light that is reflected from the tissue). FIG. 7D illustrates locations where emitter(s) may be arranged (see 2722) and locations where detectors may be arranged (see 2721). Cover(s) (for example, transparent covers) may be arranged between the detector(s) and the tissue at regions 2721 and between the emitter(s) and the tissue at regions 2722.

FIG. 7C illustrates a top view, FIG. 7D illustrates a bottom view, FIGS. 7E-7F illustrate front and rear views, and FIGS. 7G-7H illustrate side views of the electronic device 2700. As shown in FIGS. 7C-7H, the housing 2710 can include a first side 2730, a second side 2731, a third side 2732, and a fourth side 2733. The first side 2730 can connect to the top portion 2711 along a first edge of the housing 2710. The second side 2731 can connect to the top portion 2711 along a second edge of the housing 2710. The third side 2732 can connect to the top portion 2711 along a third edge of the housing 2710. The fourth side 2733 can connect to the top portion 2711 along a fourth edge of the housing 2710. The housing 2710 can include an exterior surface that is visible in at least FIGS. 7A-7H and an interior surface. The exterior and interior surface can extend along the top portion 2711, bottom portion 2713, first side 2730, second side 2731, third side 2732, and fourth side 2733.

As shown in FIGS. 7I-7K, the housing 2710 can include a first shell 2740 (which also may be referred to as a “first portion” or “top shell” of the housing 2710) and a second shell 2750 (which also may be referred to as “a second portion” or “bottom shell” of the housing 2710). The first shell 2740 and the second shell 2750 can form an interior of the housing 2710 when joined together. In some implementations, the first and second shells 2740, 2750 are permanently secured together. The electronic device 2700 can be positioned by a wearable device as described herein and/or as described in U.S. Patent Pub. No. 2024/0081656 incorporated by reference herein (for example, as described with respect to any of the wearable devices disclosed herein and/or in U.S. Patent Pub. No. 2024/0081656 incorporated by reference herein) such that shell 2750 can be placed against a tissue site of the subject (for example, against skin of the subject's wrist). The first shell 2740 can include a first side 2741a, a second side 2741b, a third side 2741c, and a fourth side 2741d. The first shell 2740 can also include the top portion 2711 of the housing 2710, and such top portion 2711 can be connected to sides 2741a, 2741b, 2741c, 2741d. The first side 2741a can be opposite to the second side 2741b. The third side 2741c can be opposite to the fourth side 2741d. The first side 2741a can be generally parallel to the second side 2741b. The third side 2741c can be generally parallel to the fourth side 2741d. The first side 2741a and the second side 2741b can each be generally perpendicular to the third side 2741c and the fourth side 2741d.

FIGS. 7I-7J illustrate top and bottom perspective views, respectively, of electronic device 2700 with shell 2740 spaced from shell 2750 and other components of electronic device 2700. FIG. 7K illustrates an enlarged top perspective view of electronic device 2700 with shell 2740 removed. The housing 2710 can be configured to house various components of the electronic device 2700. The electronic device 2700 can include spring contacts 2760, an NFC antenna 2761 (which can be an implementation and component of communication component(s) 2203), an antenna 2770 (which can be an implementation and component of communication component(s) 2203), status LEDs 2762 (which can be an implementation of status indicator(s) 2206), an audible alert module 2763 (which can be an implementation and component of other component(s) 2211), a vibration motor 2764 (which can be an implementation and component of other component(s) 2211), a battery 2765 (which can be an implementation of battery 2204), and/or a printed circuit board (PCB) 2766, and any or all of such components can be arranged within an interior of the housing 2710. Various components housed within the interior of the housing 2710 can be mounted on or arranged by the PCB 2766. PCB 2766 can include one or more processors, which can be an implementation of processor(s) 2201. The spring contacts 2760 can contact feed points on the antenna 2770, such as first feed point 2773a and second feed point 2773b, which can be arranged on an interior surface of the housing 2710 along portions of shell 2740 as discussed in more detail below. The spring contacts 2760 can facilitate electrical communication between the PCB 2766 and antenna 2770, which in turn can enable communication between one or more processors mounted to the PCB 2766 and antenna 2770. The spring contacts 2760 can provide an electrical signal to the antenna 2770 for facilitating wireless communication with external device(s) (for example, over a wireless protocol) and/or a wearable device (for example, a wearable device described herein), and/or transmitting physiological data to external device(s).

The NFC antenna 2761 can allow the electronic device 2700 to communicate with other devices when the electronic device 2700 is within an NFC range. In some implementations, NFC antenna 2761 is disposed on a portion of an interior surface of the housing 2710, for example, interior surface 2740a of first shell 2740 (see FIG. 7J). The status LEDs 2762 can emit light indicating a status of the electronic device 2700. In some examples, the status LEDs 2762 can provide light to the subject via the status indicators 2712. The audible alert module 2763 can produce an audible alert to notify the subject. For example, the audible alert may correspond to a change in a measured physiological parameter. The vibration motor 2764 can provide a vibration to notify the subject. For example, the vibration may correspond to a reminder to the user. The battery 2765 can power the electronic device 2700. In some implementations, the battery 2765 is rechargeable. The battery 2765 can be charged/recharged by connecting the electronic device 2700 to a source of electrical power. For example, the electronic device 2700 can include a charge port 2723 configured to charge/recharge the battery 2765. The PCB 2766 may provide a substrate to attach the components as described herein.

With reference to FIG. 7N, the first shell 2740 can include a corner 2743a (which may also be referred to as a “first corner” of shell 2740), a corner 2743b (which may also be referred to as a “second corner” of shell 2740), a corner 2743c (which may also be referred to as a “third corner” of shell 2740), and a corner 2743d (which may also be referred to as a “fourth corner” of shell 2740). The first corner 2743a can be defined at an intersection of the first side 2741a and the fourth side 2741d. The second corner 2743b can be defined at an intersection of the fourth side 2741d and the second side 2741b. The third corner 2743c can be defined at an intersection of the second side 2741b and the third side 2741c. The fourth corner 2743d can be defined at an intersection of the third side 2741c and the first side 2741a. Since shell 2740 forms part of housing 2710, corners 2743a, 2743b, 2743c, and 2743d can represent corners of housing 2710 as well.

With refence to FIG. 7I, shell 2740 can include a first edge 2799a, a second edge 2799b, a third edge 2799c, and a fourth edge 2799d. First edge 2799a can be defined along the intersection of the first side 2741a and the top portion 2711 Second edge 2799b can be defined along the intersection of the second side 2741b and the top portion 2711. Third edge 2799c can be defined along the intersection of the third side 2741c and the top portion 2711. Fourth edge 2799d can be defined along the intersection of the fourth side 2741d and the top portion 2711. Since shell 2740 forms part of housing 2710, edges 2799a, 2799b, 2799b, and 2799d can represent edges of housing 2710 as well.

As discussed previously, housing 2710 can include an exterior surface and an interior surface. Portions of the exterior and interior surfaces of the housing 2710 can be defined by shell 2740. FIGS. 7L-7M illustrate perspective views of shell 2740 while FIG. 7N illustrate a bottom view of shell 2740. Shell 2740 includes an interior surface 2740a (which forms at least part of an interior surface of housing 2710) and an exterior surface 2740b (which forms at least part of an exterior surface of housing 2710).

As described previously, electronic device 2700 can include antenna 2770. The antenna 2770 can be configured to allow the electronic device 2700 to wirelessly communicate with one or more separate devices. In some implementations, such as that illustrated in the figures, antenna 2770 is disposed on interior surface 2740a of shell 2740 (which, as described previously, forms at least part of an interior surface of housing 2710). Antenna 2770 can extend along one or more portions of interior surface 2740a. Antenna 2770 can extend along the interior surface 2740a and along portion(s) of the top portion 2711 and/or portion(s) of one or more of sides 2741a, 2741b, 2741c, and 2741d. Antenna 2770 can extend along one or more of edges 2799a, 2799b, 2799c, 2799d.

In some implementations, the antenna 2770 extends along the interior surface 2740a and at least some of the corners 2743a-d. In some implementations, the antenna 2770 extends along the interior surface 2740a at least partially along each of the first edge 2799a, second edge 2799b, third edge 2799c, and fourth edge 2799d. In some instances, substantially all of the antenna 2770 is disposed along portions of the interior surface 2740a along portions of the first, second, third, and fourth edges, 2799a, 2799b, 2799c, 2799d.

With reference to FIG. 7N, in some implementations, antenna 2770 includes a plurality of distinct portions. For example, in some implementations, antenna 2770 includes a first antenna leg 2771 and a second antenna leg 2772 disconnected from one another. First antenna leg 2771 can include a first end 2771a and a second end 2771b. Second antenna leg 2772 can include a first end 2772a and a second end 2772b. In some implementations, first antenna leg 2771 includes a first feed point 2773a and second antenna leg 2772 includes a second feed point 2773b. In some implementations, ends 2771b and 2772b are separated from one another by a gap 2775. In some implementations, ends 2771a and 2772a are separated from one another by a gap 2774. In some implementations, the antenna 2770 is a dipole antenna.

In some implementations, the antenna 2770 comprises a width (for example, along any of legs 2771, 2772) that is between about 1.0 millimeters (mm) and about 10.0 mm. In some implementations, antenna 2770 has a width that is no greater than about 10 mm. In some implementations, the width of the antenna 2770 is between approximately 1 mm and approximately 10 mm, for example, between approximately 2 mm and approximately 9 mm, between approximately 3 mm and approximately 8 mm, between approximately 4 mm inch and approximately 6 mm, between approximately 4.5 mm and approximately 5.5 mm, approximately 5 mm, or any value or range between any of these values or ranges or any value or range bounded by any combination of these values, although values or ranges outside these values or ranges can be used in some cases. In some implementations, the width of the antenna 2770 is uniform throughout the length of the antenna 2770. In some implementations, the width of the antenna 2770 is various throughout a length of the antenna 2770.

With reference to FIG. 7N, in some implementations, the first antenna leg 2771 is disposed on the interior surface 2740a and extends along the first edge 2799a from the first corner 2743a at least to the fourth corner 2743d. In some implementations, the first antenna leg 2771 is disposed on the interior surface 2740a and extends along an entirety of the first edge 2799a. In some implementations, the first antenna leg 2771 is disposed on the interior surface 2740a and extends through the first corner 2743a. In some implementations, the first antenna leg 2771 is disposed on the interior surface 2740a and extends through the fourth corner 2743d. In some implementations, the first antenna leg 2771 is disposed on the interior surface 2740a and extends along a portion of the third edge 2799c. In some implementations, the first antenna leg 2771 is disposed on the interior surface 2740a and extends along a portion of the fourth edge 2799d. In some implementations, a portion of the first antenna leg 2771 that extends along the third edge 2799c is longer than a portion of the first antenna leg 2771 that extends along the fourth edge 2799d. In some implementations, a portion of the first antenna leg 2771 that extends along the fourth edge 2799d is longer than a portion of the first antenna leg 2771 that extends along the third edge 2799c. In some implementations, the first antenna leg 2771 is disposed on the interior surface 2740a and extends at least partially along each of the first edge 2799a, the third edge 2799c, and the fourth edge 2799d.

With reference to FIG. 7N, in some implementations, the second antenna leg 2772 is disposed on, the interior surface 2740a and extends along the second edge 2799b from the second corner 2743b at least to the third corner 2743c. In some implementations, the second antenna leg 2772 is disposed on the interior surface 2740a and extends along an entirety of the second edge 2799b. In some implementations, the second antenna leg 2772 is disposed on the interior surface 2740a and extends through the second corner 2743b. In some implementation, the second antenna leg 2772 is disposed on the interior surface 2740a and extends through the third corner 2743c. In some implementations, the second antenna leg 2772 is disposed on the interior surface 2740a and extends along a portion of the third edge 2799c. In some examples, the second antenna leg 2772 is disposed on the interior surface 2740a and extends along portion of the fourth edge 2799d. In some instances, a portion of the second antenna leg 2772 that extends along the third edge 2799c is longer than a portion of the second antenna leg 2772 that extends along the fourth edge 2799d. In some implementations, a portion of the second antenna leg 2772 that extends along the fourth edge 2799d is longer than a portion of the second antenna leg 2772 that extends along the third edge 2799c. In some implementations, the second antenna leg 2772 is disposed on the interior surface 2740a and extends at least partially along each of the second edge 2799b, the third edge 2799c, and the fourth edge 2799d.

As discussed previously, in some examples, the first antenna leg 2771 and the second antenna leg 2772 are separated from one another, for example, via gaps 2774, 2775 (see FIG. 7N). In some implementations, the first end 2771a and the first end 2772a are disposed on interior surface 2740a along the fourth edge 2799d. In some implementations, the second end 2771b and the second end 2772b are disposed on interior surface 2740a along the third edge 2799c. The first antenna leg 2771 may comprise a length and the second antenna leg 2772 may comprise a length that is substantially equal to the length of the first antenna leg 2771. In some implementations, the first gap 2774 and the second gap 2775 are not aligned with one another (see FIG. 7N).

The first gap 2774 can comprise a width that is between about 0.0 millimeters (mm) and about 10.0 mm. The first gap 2774 can include a width that is not greater than about 10 mm. In some implementations, the width of the first gap 2774′ is between approximately 0 mm and approximately 10 mm, for example, between approximately 0.5 mm and approximately 9.5 mm, between approximately 1 mm and approximately 9 mm, between approximately 1.5 mm and approximately 8 mm, between approximately 2 mm and approximately 8 mm, between approximately 2 mm and approximately 7.5 mm, between approximately 3 mm and approximately 7 mm, between approximately 3.5 mm and approximately 6.5 mm, between approximately 4 mm and approximately 6 mm, between approximately 4.5 mm and approximately 5.5 mm, between approximately 4.5 mm and approximately 5.5 mm, approximately 5 mm, or any value or range between any of these values or ranges or any value or range bounded by any combination of these values, although values or ranges outside these values or ranges can be used in some cases.

The width of the second gap 2775 can influence the dominant resonance frequency of the antenna 2770. The width of the second gap 2775 can be adjusted to target a specific dominant resonance frequency for the antenna 2770. In some implementations, the second gap 2775 comprises a width that is between about 0 millimeters (mm) and about 100 mm. The second gap 2775 can include a width that is no greater than about 100 mm. In some implementations, the width of the second gap 2775 is between approximately 0 mm and approximately 10 mm, for example, between approximately 0.5 mm and approximately 9.5 mm, between approximately 1 mm and approximately 9 mm, between approximately 1.5 mm and approximately 8.5 mm, between approximately 2 mm and approximately 8 mm, between approximately 2.5 mm and approximately 7.5 mm, between approximately 3 mm and approximately 7 mm, between approximately 3.5 mm and approximately 6.5 mm, between approximately 4 mm and approximately 6 mm, between approximately 4.5 mm and approximately 5.5 mm, approximately 5 mm, or any value or range between any of these values or ranges or any value or range bounded by any combination of these values, although values or ranges outside these values or ranges can be used in some cases. In some implementations, the second gap 2775 comprises a width that is greater than 10 mm.

With continued reference to FIG. 7N, the width of the second gap 2775 can be greater than the width of the first gap 2774. For example, the width of the second gap 2775 can be greater than the width of the first gap 2774 by at least approximately 0.2 mm, at least approximately 0.4 mm, at least approximately 0.6 mm, at least approximately 0.8 mm, at least approximately 1 mm, at least approximately 1.2 mm, at least approximately 1.4 mm, at least approximately 1.6 mm, at least approximately 1.8 mm, at least approximately 2 mm, at least approximately 2.2 mm, at least approximately 2.4 mm, at least approximately 2.6 mm, at least approximately 2.8 mm, at least approximately 3 mm, at least approximately 3.2 mm, at least approximately 3.4 mm, at least approximately 3.6 mm, at least approximately 3.8 mm, at least approximately 4 mm, at least approximately 4.2 mm, at least approximately 4.4 mm, at least approximately 4.6 mm, at least approximately 4.8 mm, at least approximately 5 mm, at least approximately 5.2 mm, at least approximately 5.4 mm, at least approximately 5.6 mm, at least approximately 5.8 mm, at least approximately 6 mm, at least approximately 6.2 mm, at least approximately 6.4 mm, at least approximately 6.6 mm, at least approximately 6.8 mm, at least approximately 7 mm, at least approximately 7.2 mm, at least approximately 7.4 mm, at least approximately 7.6 mm, at least approximately 7.8 mm, at least approximately 8.0 mm, at least approximately 8.2 mm, at least approximately 8.4 mm, at least approximately 8.6 mm, at least approximately 8.8 mm, at least approximately 9.0 mm, although values or ranges outside these values or ranges can be used in some cases. In some variants, the width of the first gap 2774′ is greater than the width of the second gap 2775 by the values as disclosed above. A ratio between the width of the second gap 2775 and the width of the first gap 2774 can be between approximately 1 and approximately 5, for example, between approximately 1.5 and approximately 4.5, between approximately 2 and approximately 4, between approximately 2.5 and approximately 3.5, between approximately 2 and approximately 2.5, or between approximately 3.5 and approximately 4, or any value or range between any of these values or ranges or any value or range bounded by any combination of these values, although values or ranges outside these values or ranges can be used in some cases.

Antenna 2770 can be a dipole antenna. Antenna 2770 can be painted on the interior surface of the housing 2710 (for example, interior surface 2740a of shell 2740) using a laser direct structuring (LDS) technique, a laser enhanced plating (LEP) technique, or another technique for applying a conductive trace to a surface. In some implementations, the antenna 2761 is painted with one or more metallic materials, such as at least one of gold and copper.

It may be advantageous to limit a size of the electronic device 2700 to increase comfort and wearability for a user. However, minimizing the size of the electronic device 2700 results in less space available for electronic components of the electronic device 2700. In some implementations, the distance between circuit board 2766 and the top portion 2711 of housing 2710 is between about 0 millimeters (mm) and about 100 mm, for example, between approximately 0 mm and approximately 30 mm, for example, between approximately 2 mm and approximately 28 mm, between approximately 4 mm and approximately 26 mm, between approximately 6 mm inch and approximately 24 mm, between approximately 8 mm and approximately 22 mm, between approximately 10 mm and approximately 20 mm, between approximately 12 mm and approximately 18 mm, between approximately 14 mm and approximately 16 mm, approximately 15 mm, or any value or range between any of these values or ranges or any value or range bounded by any combination of these values. In some implementations, the distance between circuit board 2766 and the top portion 2711 of housing 2710 is no greater than about 30 mm. Antenna 2770 can be disposed along portions of the interior surface of the housing 2710 to protect the antenna 2770 (as opposed to being disposed on an exterior surface of the housing 2710). Antenna 2770 can be disposed along the interior surface in a manner that allows the antenna 2770 to be spaced from various other electronic components of electronic device 2700. Such arrangement can advantageously reduce interference with electronics of electronic device 2700, thereby increasing wireless range performance (e.g., Bluetooth Low Energy (BLE) performance). Antenna 2770 can be painted on the interior surface and extend along various portions of the interior surface of the housing 2710 such as any of those illustrated and/or described with respect to FIG. 7N. For example, antenna 2770 can be painted along one or more of edges 2799a, 2799b, 2799c, 2799d (and/or portions of top portion 2711 and one or more of sides 2741a, 2741b, 2741c, 2741d). Such layout of antenna 2770 can allow electronic device 2700 to have a reduced profile compared with electronic devices having other types of antennas (such as, a PIFA printed in the middle of the device), which would require the structure to extend outwards from a circuit board to give the antenna elevation away from the circuit board in order to radiate adequately. Such layout of antenna 2770 can also free up more interior space within housing 2710, thereby allowing for inclusion of a larger battery and/or other components (such as a large metal motor, such as a vibration motor) within housing 2710 of electronic device 2700.

Antenna 2770 can be spaced a distance from bottom portion 2713 of electronic device 2700. This advantageously can reduce interference of the wireless signal (produced by antenna 2770) when electronic device 2700 is secured to a user, for example, via a band such as any of those disclosed herein. Antenna 2770 can be arranged along the interior surface of housing 2710 (for example, along interior surface 2740a of shell 2740) such that antenna 2770 is spaced a distance away from a surface of a user's body when electronic device 2700 is secured thereto. Such distance may be between about 0.0 millimeters (mm) and about 100 mm, for example, between approximately 0 mm and approximately 30 mm, between approximately 2 mm and approximately 28 mm, between approximately 4 mm and approximately 26 mm, between approximately 6 mm inch and approximately 24 mm, between approximately 8 mm and approximately 22 mm, between approximately 10 mm and approximately 20 mm, between approximately 12 mm and approximately 18 mm, between approximately 14 mm and approximately 16 mm, approximately 15 mm, or any value or range between any of these values or ranges or any value or range bounded by any combination of these values. As another example, such distance may be no greater than about 30 mm.

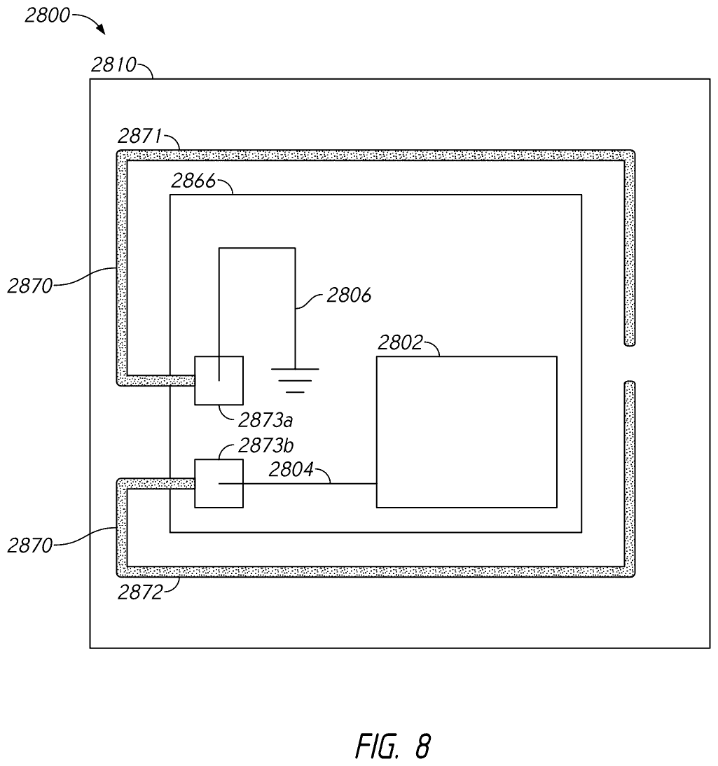

FIG. 8 illustrates a schematic diagram of an example electronic device with an antenna system. Schematically illustrated are electronic device 2800, communication module 2802, feed port 2804, ground port 2806, housing 2810, PCB 2866, antenna 2870, first antenna leg 2871, second antenna leg 2872, first feed point 2873a, and second feed point 2873b. Electronic device 2700 can include any of the features described with respect to electronic device 2800, and vice versa. The communication module 2802 can allow the electronic device 2800 to communicate via radio frequency (RF) communications. For example, the communication module 2802 may allow the electronic device 2800 to transmit information regarding measurement of the subject to other devices. In some examples, the communication module 2802 can convert analog signal measurements of physiological parameters to an RF signal for transmitting via the antenna 2870. The RF signal can include a signal varying in frequency (and/or time, and/or phase) according to a modulation type. In some instances, the feed port 2804 enables the communication module 2802 to transmit the RF signal to the antenna 2870. The feed port 2804 can connect the communication module 2802 with the second feed point 2873b. In some implementations, the feed port 2804 can provide the second antenna leg 2872 with the RF signal. In some implementations, the feed port 2804 can connect the communication module 2802 with the first feed point 2873a. In this manner, the feed port 2804 can provide the first antenna leg 2871 with the RF signal. In some implementations, the ground port 2806 can provide the first antenna leg 2871 with grounding capabilities. In some implementations, the ground port 2806 provides the second antenna leg 2872 with grounding capabilities. In some implementations, the ground port 2806 is disconnected from the feed port 2804. In some implementations, the feed port 2804 is connected directly to the communication module 2802 without an attachment to a ground along a path between the second feed point 2873b and the communication module 2802.

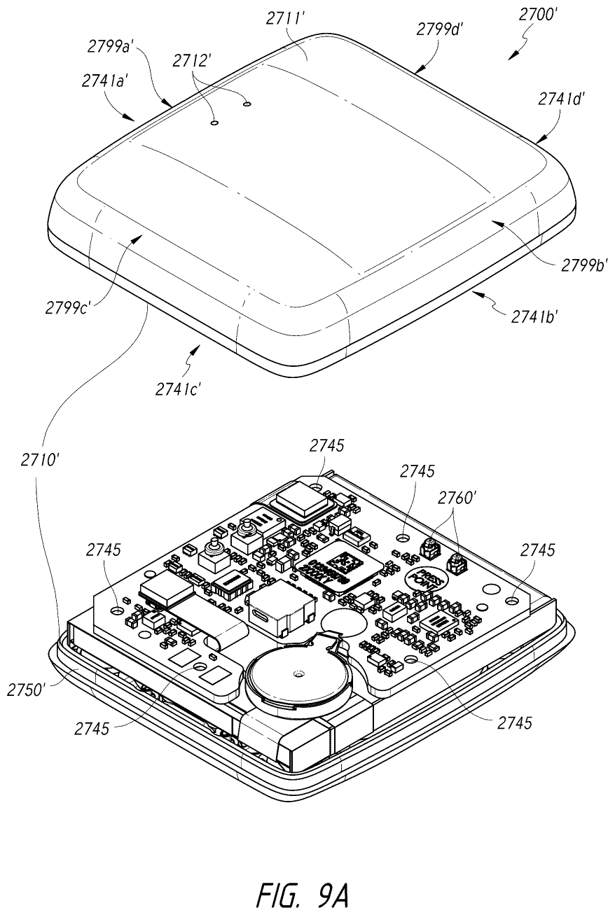

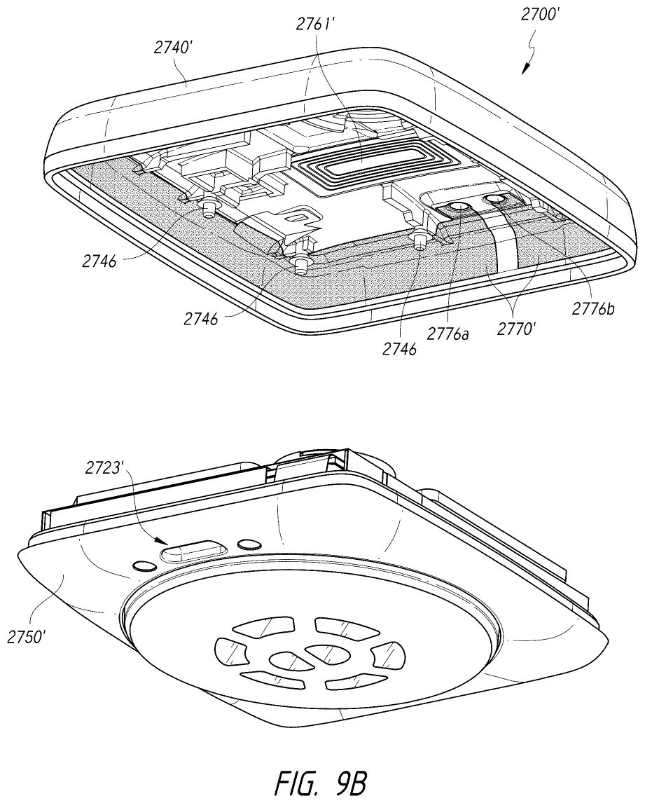

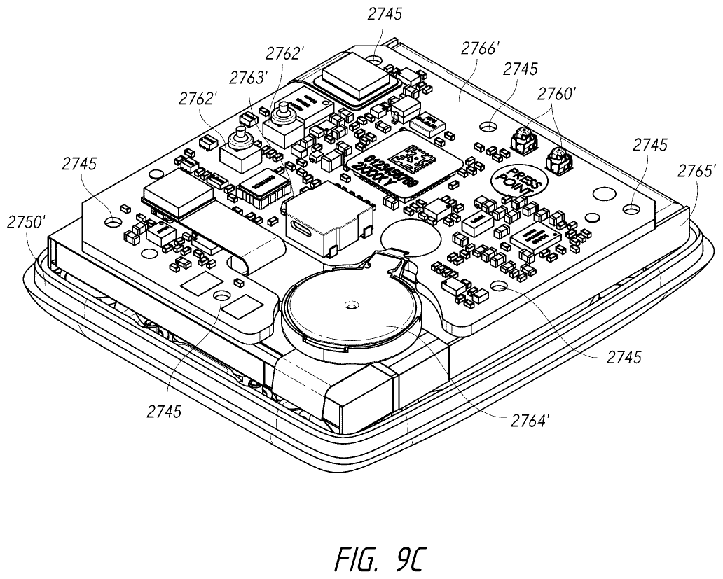

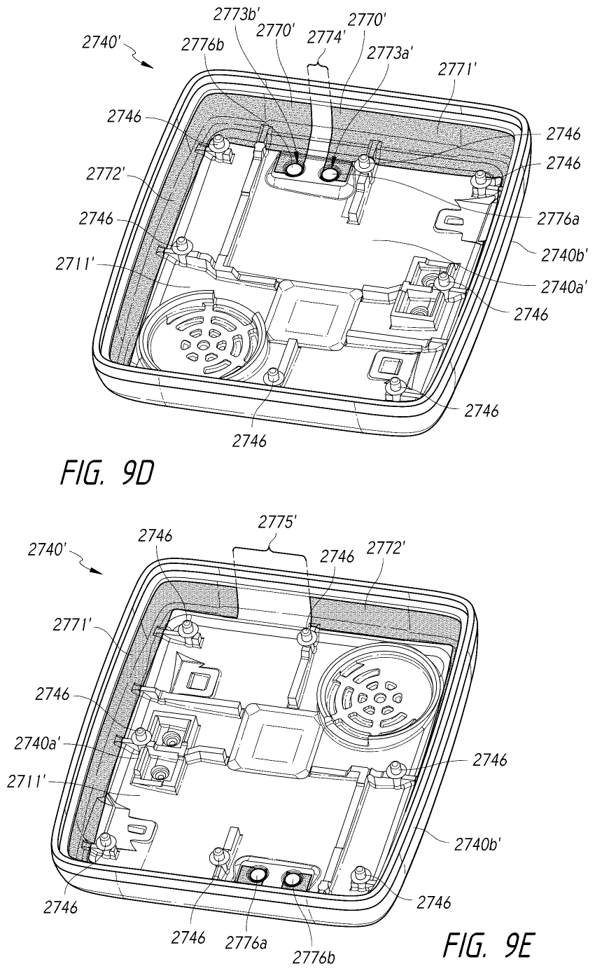

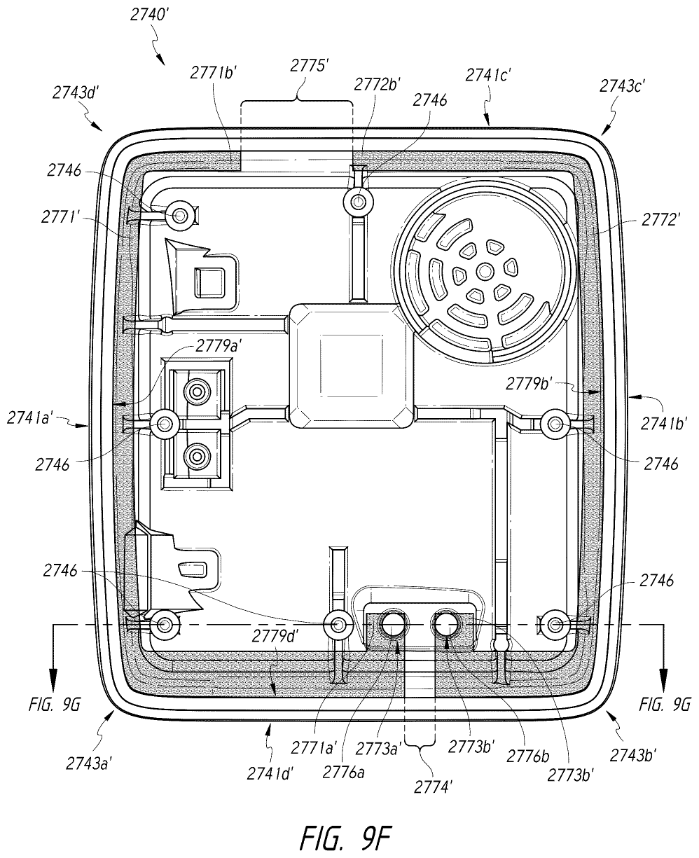

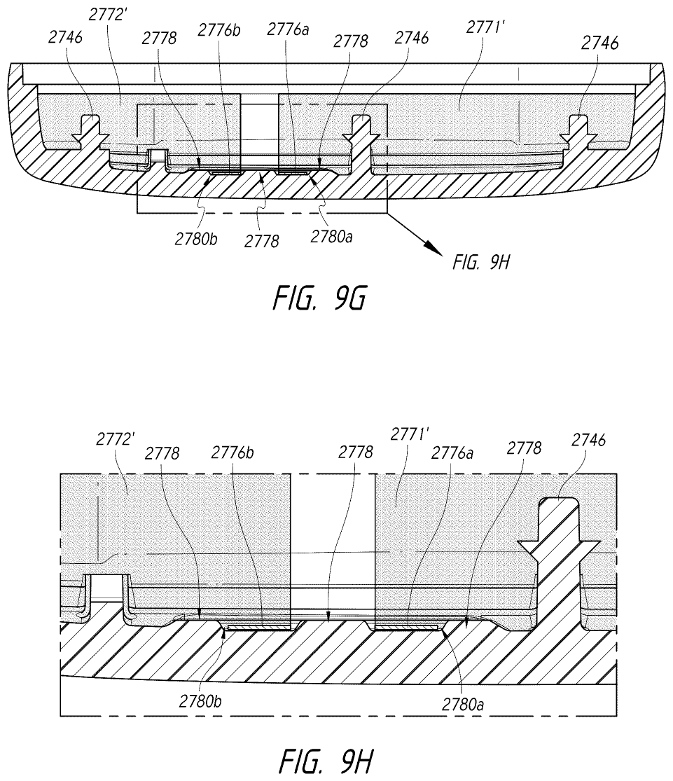

FIGS. 9A-9H illustrate various views of an electronic device 2700′. Electronic device 2700′ can be incorporated into and/or can interact with (for example, removably secure to) any of the wearable devices as described herein and/or with any of the wearable devices described in U.S. Patent Pub. No. 2024/0081656 incorporated by reference herein. Electronic device 2700′ can interact in a similar or identical manner with any of the wearable devices described herein and/or in U.S. Patent Pub. No. 2024/0081656 incorporated by reference herein as described with respect to any of the other electronic devices disclosed herein and/or in U.S. Patent Pub. No. 2024/0081656 incorporated by reference herein.

The electronic device 2700′ can include any one or more of the features illustrated and/or discussed with respect to FIG. 2F, for example, the one or more processors 1201, the one or more storage devices 1202, the communication module 1203, the battery 1204, the information element 1205, the one or more status indicators 1206, the one or more emitters 1207, the one or more detectors 1208, the one or more ECG electrodes 1209, the one or more other sensors 1210, and/or the one or more other components 1211 disclosed in U.S. Patent Pub. No. 2024/0081656 incorporated by reference herein. Furthermore, the electronic device 2700′ and/or any of the electronic devices described herein (for example, including electronic devices 2700 and/or 3200) can be similar or identical to and/or incorporate any of the features described and/or illustrated with respect to any of the devices, assemblies, and/or systems described and/or illustrated in U.S. Pat Pub. No. 2021/0290120, U.S. Pat. Pub. No. 2023/0028745, and U.S. Pat. Pub. No. 2024/0188872 incorporated by reference herein.

The electronic device 2700′ can include any one or more of the features illustrated and/or discussed with respect to FIG. 6, for example, the processor(s) 2201, the storage component(s) 2202, the communication component(s) 2203, the battery 2204, the status indicator(s) 2206, the emitter(s) 2207, the detector(s) 2208, the ECG electrode(s) 2209, the other sensor(s) 2210, and/or the other component(s) 2211. Furthermore, the electronic device 2700′ can be similar or identical to and/or incorporate any of the features described and/or illustrated with respect to any of the devices, assemblies, and/or systems described and/or illustrated in U.S. Patent Pub. No. 2024/0081656 incorporated by reference herein. Electronic device 2700′ can be similar to the electronic device 2700 in several respects. For example, electronic device 2700′ can include a housing 2710′ which can be similar or identical to housing 2710′, status indicators 2712′ which can be similar or identical to the status indicators 2712, and a charge port 2723′ which can be similar or identical to the charge port 2723.

As shown in FIGS. 9A-9C, the housing 2710′ can include a first shell 2740′ (which also may be referred to as a “first portion” or “top shell” of the housing 2710′) and a second shell 2750′ (which also may be referred to as “a second portion” or “bottom shell” of the housing 2710′). The first shell 2740′ and the second shell 2750′ can form an interior of the housing 2710′ when joined together. In some implementations, the first and second shells 2740′. 2750′ are permanently secured together. The electronic device 2700′ can be positioned by a wearable device as described herein and/or as described in U.S. Patent Pub. No. 2024/0081656 incorporated by reference herein (for example, as described with respect to any of the wearable devices disclosed herein and/or in U.S. Patent Pub. No. 2024/0081656 incorporated by reference herein) such that shell 2750′ can be placed against a tissue site of the subject (for example, against skin of the subject's wrist). The first shell 2740′ can include a first side 2741a′, a second side 2741b′, a third side 2741c′, and a fourth side 2741d′. The first shell 2740′ can also include the top portion 2711′ of the housing 2710′, and such top portion 2711′ can be connected to sides 2741a′, 2741b′, 2741c′, 2741d′. The first side 2741a′ can be opposite to the second side 2741b′. The third side 2741c′ can be opposite to the fourth side 2741d′. The first side 2741a′ can be generally parallel to the second side 2741b′. The third side 2741c′ can be generally parallel to the fourth side 2741d′. The first side 2741a′ and the second side 2741b′ can each be generally perpendicular to the third side 2741c′ and the fourth side 2741d′.

FIGS. 9A-9B illustrate top and bottom perspective views (respectively) of electronic device 2700′ with shell 2740′ spaced from shell 2750′ and other components of electronic device 2700′. FIG. 9C illustrates an enlarged top perspective view of electronic device 2700′ with shell 2740′ removed. The housing 2710′ can be configured to house various components of the electronic device 2700′. The electronic device 2700′ can include electrical connectors 2760′(which can be pin connectors), an NFC antenna 2761′, status LEDs 2762′, an audible alert module 2763′, a vibration motor 2764′, a battery 2765′, and/or a printed circuit board (PCB) 2766′, and any or all of such components can be arranged within an interior of the housing 2710′. In some implementations, the PCB 2766′ can include a plurality of holes 2745 shaped to receive a plurality of plugs 2746 extending from the first shell 2740′. The first shell 2740′ can be secured to the PCB 2766′ when the plugs 2746 are received in the holes 2745. Various components housed within the interior of the housing 2710′ can be mounted on the PCB 2766′. The electrical connectors 2760′ can contact feed points on the antenna 2770′, such as first feed point 2773a′ and second feed point 2773b′, which can be arranged on an interior surface of the housing 2710′ along portions of shell 2740′ as discussed in more detail below. In some implementations, the electrical connectors 2760′ contact pads 2776a, 2776b on the antenna 2770′, which can be arranged on an interior surface of the housing 2710′ along portions of shell 2740′ as discussed in more detail below. The electrical connectors 2760′ can facilitate electrical communication between the PCB 2766′ and antenna 2770′, which in turn can enable communication between one or more processors mounted to the PCB 2766′ and antenna 2770′. The electrical connectors 2760′ can provide an electrical signal to the antenna 2770′ for facilitating wireless communication with an external device (e.g., over a wireless protocol) and/or transmitting physiological data to external device(s).

The NFC antenna 2761′ can allow the electronic device 2700′ to communicate with other devices when the electronic device 2700′ is within an NFC range. In some implementations. NFC antenna 2761′ is disposed on a portion of an interior surface of the housing 2710′, for example, interior surface 2740a′ of first shell 2740′ (see FIGS. 9B and 9D). The status LEDs 2762′ can emit light indicating a status of the electronic device 2700′. In some examples, the status LEDs 2762′ can provide light to the subject via the status indicators 2712′. The audible alert module 2763′ can produce an audible alert to notify the subject. For example, the audible alert may correspond to a change in a measured physiological parameter. The vibration motor 2764′ can provide a vibration to notify the subject. For example, the vibration may correspond to a reminder to the user. The battery 2765′ can power the electronic device 2700′. In some implementations, the battery 2765′ is rechargeable. The battery 2765′ can be charged/recharged by connecting the electronic device 2700′ to a source of electrical power. For example, the electronic device 2700′ can include a charge port 2723′ configured to charge/recharge the battery 2765′. The PCB 2766′ may provide a substrate to attach the components as described herein.

With reference to FIG. 9F, the first shell 2740′ can include a corner 2743a′ (which may also be referred to as a “first corner” of shell 2740′), a corner 2743b′ (which may also be referred to as a “second corner” of shell 2740′), a corner 2743c′ (which may also be referred to as a “third corner” of shell 2740′), and a corner 2743d′ (which may also be referred to as a “fourth corner” of shell 2740″). The first corner 2743a′ can be defined at an intersection of the first side 2741a′ and the fourth side 2741d′. The second corner 2743b′ can be defined at an intersection of the fourth side 2741d′ and the second side 2741b′. The third corner 2743c′ can be defined at an intersection of the second side 2741b′ and the third side 2741c′. The fourth corner 2743d′ can be defined at an intersection of the third side 2741c′ and the first side 2741a′. Since shell 2740′ forms part of housing 2710′, corners 2743a′, 2743b′, 2743c′, and 2743d′ represent corners of housing 2710′ as well.

With refence to FIG. 9A, shell 2740′ can include a first edge 2799a′, a second edge 2799b, a third edge 2799c′, and a fourth edge 2799d′. First edge 2799a′ can be defined along the intersection of the first side 2741a′ and the top portion 2711′. Second edge 2799b′ can be defined along the intersection of the second side 2741b′ and the top portion 2711′. Third edge 2799c′ can be defined along the intersection of the third side 2741c′ and the top portion 2711′. Fourth edge 2799d′ can be defined along the intersection of the fourth side 2741d′ and the top portion 2711′. Since shell 2740′ forms part of housing 2710′, edges 2799a′, 2799b′, 2799b′, and 2799d′ can represent edges of housing 2710′ as well.

As discussed previously, housing 2710′ can include an exterior surface and an interior surface. Portions of the exterior and interior surfaces of the housing 2710′ can be defined by shell 2740′. FIGS. 9D-9E illustrate perspective views of shell 2740′ while FIG. 9F illustrate a bottom view of shell 2740′. Shell 2740′ includes an interior surface 2740a′ (which forms at least part of an interior surface of housing 2710′) and an exterior surface 2740b′ (which forms at least part of an exterior surface of housing 2710′).

As described previously, electronic device 2700′ can include antenna 2770′. The antenna 2770′ can be configured to allow the electronic device 2700′ to wirelessly communicate with one or more separate devices. In some implementations, such as that illustrated in the figures, antenna 2770′ is disposed on interior surface 2740a′ of shell 2740′ (which, as described previously, forms at least part of an interior surface of housing 2710′). Antenna 2770′ can extend along one or more portions of interior surface 2740a′. Antenna 2770′ can extend along the interior surface 2740a′ and along portion(s) of the top portion 2711′ and/or portion(s) of one or more of sides 2741a′, 2741b′, 2741c′, and 2741d′. Antenna 2770′ can extend along one or more of edges 2799a′, 2799b, 2799c′, 2799d′.

In some implementations, the antenna 2770′ extends along the interior surface 2740a′ and at least some of the corners 2743a-d′. In some implementations, the antenna 2770′ extends along the interior surface 2740a′ at least partially along each of the first edge 2799a′, second edge 2799b′, third edge 2799c′, and fourth edge 2799d′. In some instances, substantially all of the antenna 2770′ is disposed along portions of the interior surface 2740a′ along portions of the first, second, third, and fourth edges, 2799a′, 2799b′, 2799c′, 2799d′.

With reference to FIG. 9F, in some implementations, antenna 2770′ includes a plurality of distinct portions. For example, in some implementations, antenna 2770′ includes a first antenna leg 2771′ and a second antenna leg 2772′ disconnected from one another. First antenna leg 2771′ can include a first end 2771a′ and a second end 2771b″. Second antenna leg 2772′ can include a first end 2772a′ and a second end 2772b′. In some implementations, first antenna leg 2771′ includes a first feed point 2773a′ and second antenna leg 2772′ includes a second feed point 2773b′. In some implementations, a first pad 2776a can be positioned adjacent the first end 2771a′ and a second pad 2776b can be positioned adjacent the second end 2771b′. In some implementations, ends 2771b′ and 2772b′ are separated from one another by a gap 2775′. In some implementations, ends 2771a′ and 2772a′ are separated from one another by a gap 2774″. In some implementations, the antenna 2770′ is a dipole antenna.

In some implementations, the antenna 2770′ comprises a width (for example, along any of legs 2771′, 2772′) that is between about 1 millimeters (mm) and about 10 mm. In some implementations, antenna 2770′ has a width that is no greater than about 10 mm. In some implementations, the width of the antenna 2770′ is between approximately 1 mm and approximately 10 mm, for example, between approximately 2 mm and approximately 9 mm, between approximately 3 mm and approximately 8 mm, between approximately 4 mm inch and approximately 6 mm, approximately 5 mm, or any value or range between any of these values or ranges or any value or range bounded by any combination of these values, although values or ranges outside these values or ranges can be used in some cases. In some implementations, the width of the antenna 2770′ is uniform throughout the length of the antenna 2770′. In some implementations, the width of the antenna 2770′ is various throughout the length of the antenna 2770′.

With reference to FIG. 9F, in some implementations, the first antenna leg 2771′ is disposed on the interior surface 2740a′ and extends along the first edge 2799a′ from the first corner 2743a′ at least to the fourth corner 2743d′. In some implementations, the first antenna leg 2771′ is disposed on the interior surface 2740a′ and extends along an entirety of the first edge 2799a′. In some implementations, the first antenna leg 2771′ is disposed on the interior surface 2740a′ and extends through the first corner 2743a′. In some implementations, the first antenna leg 2771′ is disposed on the interior surface 2740a′ and extends through the fourth corner 2743d′. In some implementations, the first antenna leg 2771′ is disposed on the interior surface 2740a′ and extends along a portion of the third edge 2799c′. In some implementations, the first antenna leg 2771′ is disposed on the interior surface 2740a′ and extends along a portion of the fourth edge 2799d′. In some implementations, a portion of the first antenna leg 2771′ that extends along the third edge 2799c′ is longer than a portion of the first antenna leg 2771′ that extends along the fourth edge 2799d′. In some implementations, a portion of the first antenna leg 2771′ that extends along the fourth edge 2799d′ is longer than a portion of the first antenna leg 2771′ that extends along the third edge 2799c′. In some implementations, the first antenna leg 2771′ is disposed on the interior surface 2740a′ and extends at least partially along each of the first edge 2799a′, the third edge 2799c′, and the fourth edge 2799d′.

With reference to FIG. 9F, in some implementations, the second antenna leg 2772′ is disposed on the interior surface 2740a′ and extends along the second edge 2799b″ from the second corner 2743b′ at least to the third corner 2743c′. In some implementations, the second antenna leg 2772′ is disposed on the interior surface 2740a′ and extends along an entirety of the second edge 2799b′. In some implementations, the second antenna leg 2772′ is disposed on the interior surface 2740a and extends through the second corner 2743b′. In some implementation, the second antenna leg 2772′ is disposed on the interior surface 2740a and extends through the third corner 2743c′. In some implementations, the second antenna leg 2772′ is disposed on the interior surface 2740a′ and extends along a portion of the third edge 2799c′. In some examples, the second antenna leg 2772′ is disposed on the interior surface 2740a′ and extends along portion of the fourth edge 2799d′. In some instances, a portion of the second antenna leg 2772′ that extends along the third edge 2799c′ is longer than a portion of the second antenna leg 2772′ that extends along the fourth edge 2799d′. In some implementations, a portion of the second antenna leg 2772′ that extends along the fourth edge 2799d′ is longer than a portion of the second antenna leg 2772′ that extends along the third edge 2799c′. In some implementations, the second antenna leg 2772′ is disposed on the interior surface 2740a′ and extends at least partially along each of the second edge 2799b′, the third edge 2799c′, and the fourth edge 2799d′.

As discussed previously, in some examples, the first antenna leg 2771′ and the second antenna leg 2772′ are separated from one another, for example, via gaps 2774′, 2775′ (see FIG. 9F). In some implementations, the first end 2771a′ and the first end 2772a′ are disposed on interior surface 2740a′ along the fourth edge 2799d′. In some implementations, the second end 2771b′ and the second end 2772b′ are disposed on interior surface 2740a′ along the third edge 2799c′. The first antenna leg 2771′ may comprise a length and the second antenna leg 2772′ may comprise a length that is substantially equal to the length of the first antenna leg 2771′. In some implementations, the first gap 2774′ and the second gap 2775′ are not aligned with one another (see FIG. 9F)

The first gap 2774′ can comprise a width that is between about 0 millimeters (mm) and about 10 mm. The first gap 2774′ can include a width that is not greater than about 10 mm. In some implementations, the width of the first gap 2774′ is between approximately 0 mm and approximately 10 mm, for example, between approximately 0.5 mm and approximately 9.5 mm, between approximately 1 mm and approximately 9 mm, between approximately 1.5 mm inch and approximately 8.5 mm, between approximately 2 mm and approximately 8 mm, between approximately 2.5 mm and approximately 7.5 mm, between approximately 3 mm and approximately 7 mm, between approximately 3.5 mm inch and approximately 6.5 mm, between approximately 4 mm and approximately 6 mm, between approximately 4.5 mm and approximately 5.5 mm, approximately 5 mm, or any value or range between any of these values or ranges or any value or range bounded by any combination of these values, although values or ranges outside these values or ranges can be used in some cases.

In some implementations, the second gap 2775′ comprises a width that is between about 0 millimeters (mm) and about 10 mm. The second gap 2775′ can include a width that is no greater than about 10 mm. In some implementations, the width of the second gap 2775′ is between approximately 0 mm and approximately 10 mm, for example, between approximately 0.5 mm and approximately 9.5 mm, between approximately 1 mm and approximately 9 mm, between approximately 1.5 mm inch and approximately 8.5 mm, between approximately 2 mm and approximately 8 mm, between approximately 2.5 mm and approximately 7.5 mm, between approximately 3 mm and approximately 7 mm, between approximately 3.5 mm inch and approximately 6.5 mm, between approximately 4 mm and approximately 6 mm, between approximately 4.5 mm and approximately 5.5 mm, between approximately 4.5 mm and approximately 5 mm, or any value or range between any of these values or ranges or any value or range bounded by any combination of these values, although values or ranges outside these values or ranges can be used in some cases. For example, in some implementations, the second gap 2775′ comprises a width that is greater than 10 mm. In some implementations, the width of the second gap 2775′ can influence the dominant resonance frequency of the antenna 2770′. In some implementations, the width of the second gap 2775′ can be adjusted to target a specific dominant resonance frequency for the antenna 2770″.

With continued reference to FIG. 9F, the width of the second gap 2775′ can be greater than the width of the first gap 2774′. For example, the width of the second gap 2775′ can be greater than the width of the first gap 2774′ by at least approximately 0.2 mm, at least approximately 0.4 mm, at least approximately 0.6 mm, at least approximately 0.8 mm, at least approximately 1 mm, at least approximately 1.2 mm, at least approximately 1.4 mm, at least approximately 1.6 mm, at least approximately 1.8 mm, at least approximately 2 mm, at least approximately 2.2 mm, at least approximately 2.4 mm, at least approximately 2.6 mm, at least approximately 2.8 mm, at least approximately 3 mm, at least approximately 3.2 mm, at least approximately 3.4 mm, at least approximately 3.6 mm, at least approximately 3.8 mm, at least approximately 4 mm, at least approximately 4.2 mm, at least approximately 4.4 mm, at least approximately 4.6 mm, at least approximately 4.8 mm, at least approximately 5 mm, at least approximately 5.2 mm, at least approximately 5.4 mm, at least approximately 5.6 mm, at least approximately 5.8 mm, at least approximately 6 mm, at least approximately 6.2 mm, at least approximately 6.4 mm, at least approximately 6.6 mm, at least approximately 6.8 mm, at least approximately 7 mm, at least approximately 7.2 mm, at least approximately 7.4 mm, at least approximately 7.6 mm, at least approximately 7.8 mm, at least approximately 8 mm, at least approximately 8.2 mm, at least approximately 8.4 mm, at least approximately 8.6 mm, at least approximately 8.8 mm, at least approximately 9 mm, although values or ranges outside these values or ranges can be used in some cases. In some variants, the width of the first gap 2774′ is greater than the width of the second gap 2775′ by the values as disclosed above. A ratio between the width of the second gap 2775′ and the width of the first gap 2774′ can be between approximately 1 and approximately 5, for example, between approximately 1.5 and approximately 4.5, between approximately 2 and approximately 4, between approximately 2.5 and approximately 3.5, between approximately 2 and approximately 2.5, between approximately 3.5 and approximately 4, or any value or range between any of these values or ranges or any value or range bounded by any combination of these values, although values or ranges outside these values or ranges can be used in some cases.

FIGS. 9G-9H illustrate cross-sectional views of shell 2740′. Shell 2740′ can include recesses 2780a, 2780b, shaped and sized to receive the pads 2776a, 2776b and/or portions of the antenna 27070′, for example, the antenna legs 2771′, 2772′. In some implementations, the recesses 2780a, 2780b and/or the pads 2776a, 2776b can be substantially circular. In some implementations, the pads 2776a, 2776b can be installed in the shell 2740′ on top of the antenna 2770′. In some implementations, the pads 2776a, 2776b can be soldered onto the antenna 2770′. In some implementations, the pads 2776a, 2776b can be compressed into the recesses 2780a, 2780b when the shell 2740′ is assembled. In some implementations, the electrical connectors 2760′ can contact the pads 2776a, 2776b on the antenna 2770′ to facilitate electrical communication between the PCB 2766′ and antenna 2770′, which in turn can enable communication between one or more processors mounted to the PCB 2766′ and antenna 2770′. In some implementations, the pads 2776a, 2776b can be made of gold which can increase the electrical conductivity of the pads 2776a, 2776b, facilitating electrical communication between the PCB 2766′ and the antenna 2770′. The pads 2776a, 2776b can reduce wear on the shell 2740′ by providing a cushion between the electrical connectors 2760′ and the shell 2740′. In some implementations, the shell 2740′ can include a plurality of protrusions 2778 that at least partially surround recesses 2780a. 2780b and/or pads 2776a, 2776b. In some implementations, the pads 2776a, 2776b can be sized such that they do not extend beyond a height of the protrusions 2778 which can reduce inadvertent contact between the pads 2776a, 2776b and components other than the electrical connectors 2760′.

Pads 2776a, 2776b (which may also be referred to as “conductive pads” or “contact pads”) can distribute forces applied by electrical connectors 2760′ against portions of antenna 2771′ (feed points 2773a′, 2773b′) and portions of the interior surface of housing 2710′ (portions of interior surface 2740a). Pads 2776a, 2776b thereby can advantageously reduce or eliminate damage to (for example, cracking of) such portions of the interior surface and of portions of antenna 2771′. This is particularly advantageous where antenna 2771′ is an LDS antenna, which may in some cases be vulnerable to cracking (e.g., eggshell cracking and/or annular cracking) due to forces applied by electrical connectors which may be exacerbated during vibrations experienced when electronic device 2700′ is in use. Pads 2776a. 2776b can advantageously mitigate these issues while at the same time facilitating electrical communication between antenna 2771′ and electrical connectors 2760′ (and in turn, between antenna 2771′ and circuit board 2766′). Pads 2776a. 2776b can be coupled to portions of antenna 2771′ (for example, feed points 2773a′, 2773b) via soldering.

Pads 2776a, 2776b can comprise one or more metallic materials, for example, at least one of gold, gold-plated over nickel, stainless steel (such as SUS-304H), copper, and/or the like. In some implementations, pads 2776a, 2776b are circular. In some implementations, pads 2776a, 2776b each have a diameter between about 1 mm and about 10 mm. In some implementations, pads 2776a, 2776b each have a diameter that is no greater than about 10 mm. In some implementations, the diameter for each of pads 2776a, 2776b is between approximately 1 mm and approximately 10 mm, for example, between approximately 2 mm and approximately 9 mm, between approximately 3 mm and approximately 8 mm, between approximately 4 mm and approximately 6 mm, approximately 5 mm, or any value or range between any of these values or ranges or any value or range bounded by any combination of these values, although values or ranges outside these values or ranges can be used in some cases. Each of the contact pads may include a thickness between about 1 mm and about 10 mm. In some implementations, the contact pads each have a thickness that is no greater than about 10 mm. In some implementations, the thickness for each of the contact pads may be between approximately 1 mm and approximately 10 mm, for example, between approximately 2 mm and approximately 9 mm, between approximately 3 mm and approximately 8 mm, between approximately 4 mm and approximately 6 mm, approximately 5 mm, or any value or range between any of these values or ranges or any value or range bounded by any combination of these values, although values or ranges outside these values or ranges can be used in some cases.

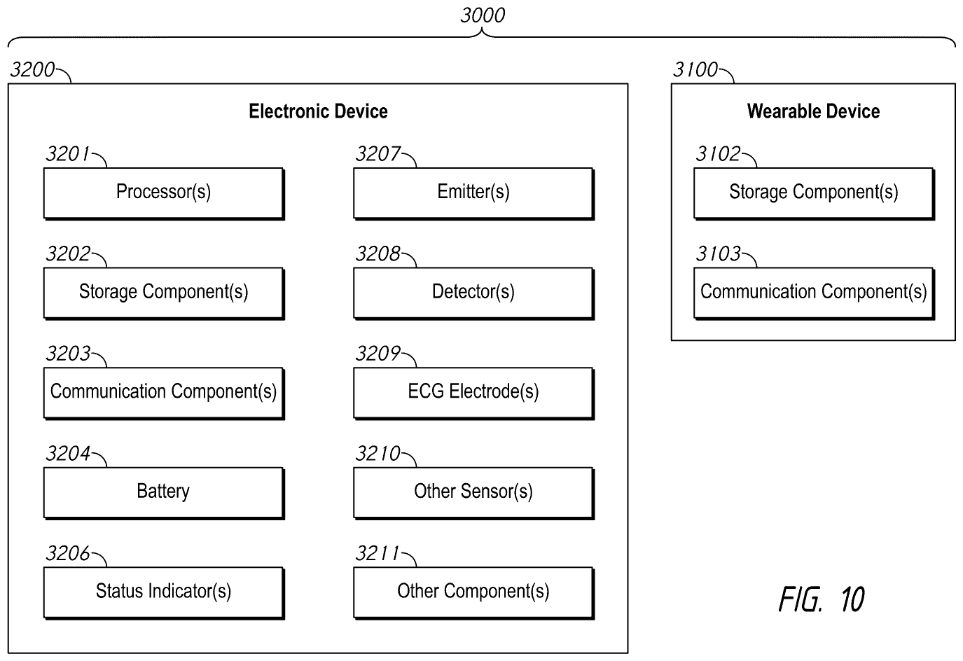

FIG. 10 illustrates a schematic diagram of certain features which can be incorporated in any of the implementations of the wearable systems and/or associated electronic devices and wearable devices described herein. As shown, a wearable system 3000 can include an electronic device 3200 and a wearable device 3100. As described herein, the electronic device 3200 can be removably secured (for example, mechanically and/or electrically) to the wearable device 3100 and as such the electronic device 3200 and wearable device 3100 can be distinct components. In some implementations, the electronic device 3200 and wearable device 3100 are integrally formed with one another. In some implementations, the electronic device 3200 and wearable device 3100 are configured to be non-separable after being joined and/or secured to one another.

The electronic device 3200 can be similar or identical to and/or incorporate any of the features described with respect to electronic device 2700, electronic device 2700′ or any of the electronic devices described in U.S. Patent Pub. No. 2024/0081656 incorporated by reference herein. For example and as shown, the electronic device 3200 can include one or more emitters 3207 and one or more detectors 3208 that are similar or identical to the emitter(s) 2207 and detector(s) 2208 described herein. Such one or more emitters 3207 and one or more detectors 3208 can form a pulse oximetry sensor of the electronic device 3200. Also shown, the electronic device 3200 can include one or more processors 3201, one or more storage components 3202 (which can also be referred to as “storage devices” or “memory”), one or more communication components 3203 (which can also be referred to as “communication module(s)”), a battery 3204, one or more status indicators 3206, one or more ECG electrodes 3209, one or more other sensors 3210, and/or one or more other components 3211 that can be similar or identical to the one or more processors 2201, the one or more storage components 2202, the one or more communication components 2203, the battery 2204, the one or more status indicators 2206, the one or more ECG electrodes 2209, the one or more other sensors 2210, and/or the one or more other components 2211 described herein. While certain features of the electronic device 3200 are shown in FIG. 10 that can be incorporated therein, any of such features are optional. Furthermore, the electronic device 3200 can include other features than shown in FIG. 10.

The wearable device 3100 can be similar or identical to and/or incorporate any of the features described with respect to any of the wearable devices described in U.S. Patent Pub. No. 2024/0081656 incorporated by reference herein. As shown, the wearable device 3100 can include one or more storage components 3102 and/or one or more communication components 3103. The storage component(s) 3102 can be similar or identical to and/or incorporate any of the features described with respect to the storage component(s) 3202 or 2202 described herein. The communication component(s) 3103 can be similar or identical to and/or incorporate any of the features described with respect to the communication component(s) 3203 or 2203 described herein. While certain features of the wearable device 3100 are shown in FIG. 10 that can be incorporated therein, any of such features are optional. For example, in some implementations the wearable device 3100 does not include communication component(s) 3103. Furthermore, the wearable device 3100 can include other features than shown in FIG. 10.

The storage component(s) 3102 can be configured to store information related to product information of the wearable device 3100. Such product information can be used in determining if the wearable device 3100 is an authorized product (for example, by the electronic device 3200). In some implementations, such product information is transmitted to the storage component(s) 3102 by the manufacturer. The storage component(s) 3102 can be configured to store patient identification data associated with a patient. As described herein, such patient identification data (which can be referred to as patient identification data 3186) can include one or more of a name, assigned identification number or health record number, date of birth, phone number, social security number, address, photo, dates of hospitalizations or encounters, name of attending physicians or care providers, demographics, diagnoses, problems list, progress notes, medications, vital signs, laboratory data, tests, allergies, immunizations, treatment plans, tag, marker, serial number, bar code, QR code, facial recognition, fingerprint recognition, voice recognition, eye recognition, gesture recognition, biometric data, data from the patient's electronic health record, or the like, which may be unique to the patient. In some implementations, such patient identification data is transmitted to the storage component(s) 3102 in a healthcare environment (for example, by a user or care provider in the healthcare environment). The storage component(s) 3102 can be configured to receive and store patient identification data prior to the wearable device 3100 being secured to the associated patient, while the wearable device 3100 is secured to the associated patient, or after the wearable device 3100 is secured to the associated patient. Furthermore, the storage component(s) 3102 can be configured to receive and store patient identification data prior to, concurrently, or after an electronic device, such as electronic device 3200, is secured thereto. In some implementations, storage component(s) 3102 can be configured to receive and store historical physiological data of the patient. In some implementations, storage component(s) 3102 can be configured to receive and store physiological data of the patient measured by the electronic device 3200.

The electronic device 3200 and the wearable device 3100 can communicate with one another. The electronic device 3200 can receive information from the wearable device 3100. The electronic device 3200 can access information from the wearable device 3100. The electronic device 3200 can transmit information to the wearable device 3100. The wearable device 3100 can transmit information to the electronic device 3200. For example, the electronic device 3200 can receive, access, and/or transmit information stored by the storage component(s) 3102 of the wearable device 3100. Such information can include patient identification data stored by the storage component(s) 3102. In some implementations, communication between electronic device 3200 and wearable device 3100 can be performed via an electrical connection therebetween (for example, via electrical contacts of each of the electronic device 3200 and the wearable device 3100). In some implementations, communication between electronic device 3200 and wearable device 3100 can be performed wirelessly therebetween (for example, via communication component(s) 3203 of the electronic device 3200 and communication component(s) 3103 of the wearable device 3100 when included). Communication between electronic device 3200 and wearable device 3100 can occur automatically or upon input from a patient or a user. Automatic communication between electronic device 3200 and wearable device 3100 can occur when the electronic device 3200 and wearable device 3100 are secured to one another (for example, when electrical contacts of each of the electronic device 3200 and the wearable device 3100 contact one another). Automatic communication between electronic device 3200 and wearable device 3100 can occur when electronic device 3200 and wearable device 3100 are within a proximity of one another (for example, when electronic device 3200 and wearable device 3100 are within a wireless communication range of one another).

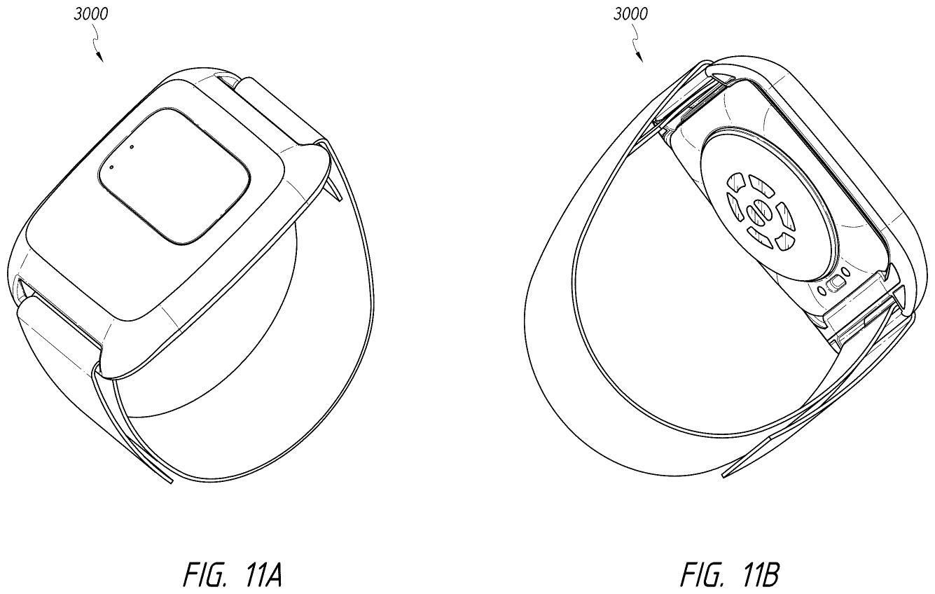

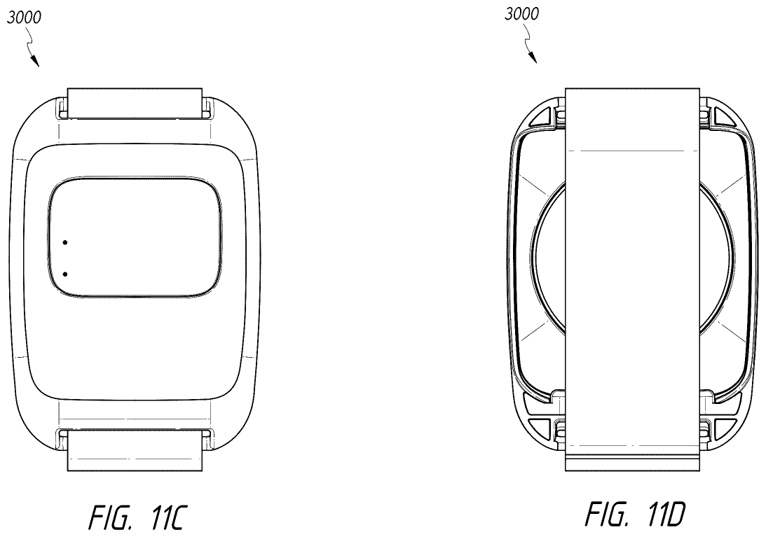

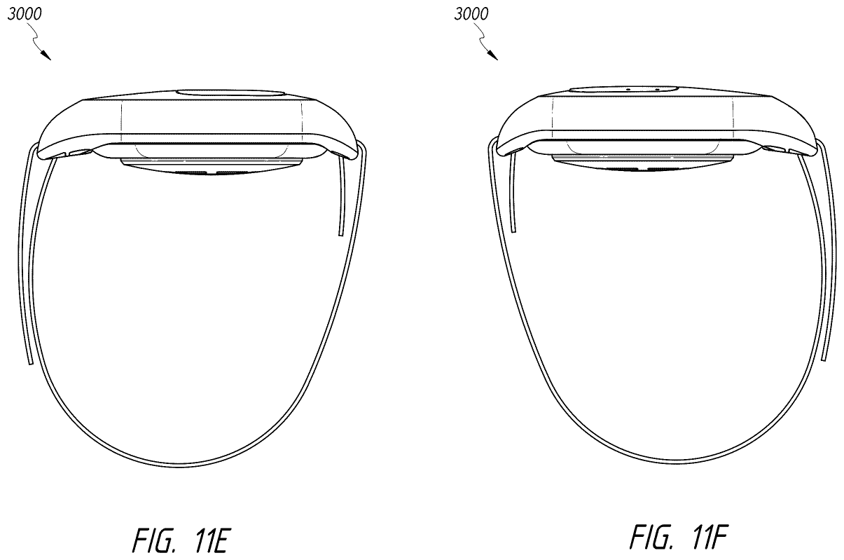

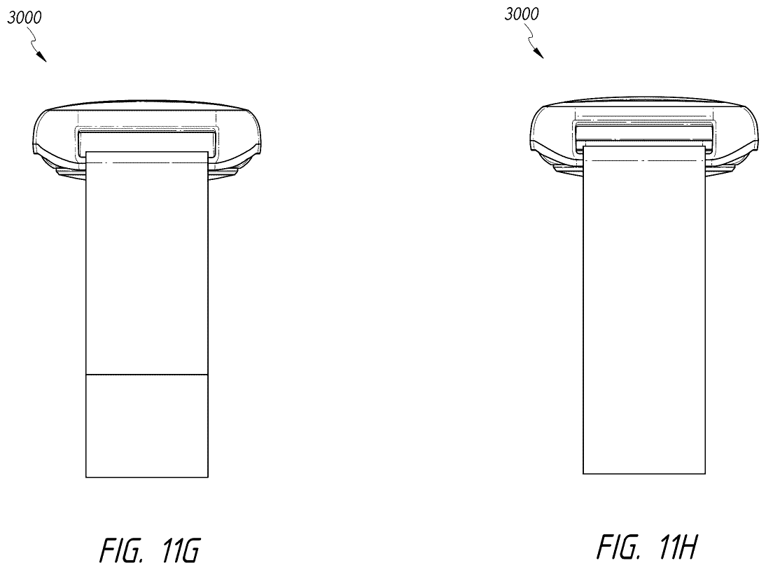

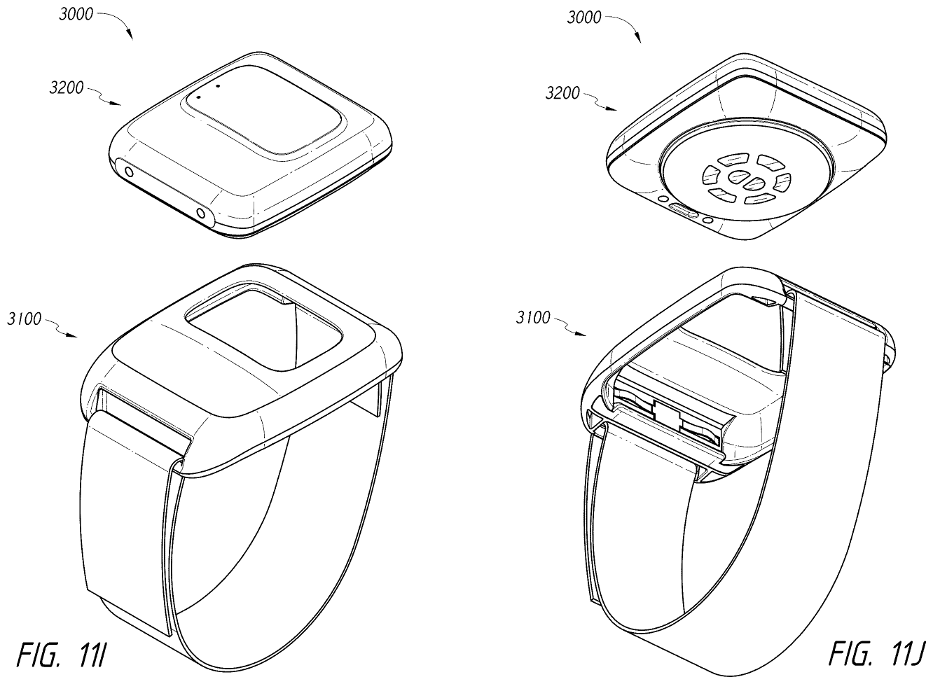

FIGS. 11A-11J illustrate various views of the wearable system 3000. FIG. 11A shows a top perspective view, FIG. 11B shows a bottom perspective view, FIG. 11C shows a top view, FIG. 11D shows a bottom view, FIGS. 11E-11F show side views, FIG. 11G shows a front view, and FIG. 11H shows a rear view, respectively, of the wearable system 3000. As described with respect to FIG. 10, wearable system 3000 can include the wearable device 3100. The wearable device 3100 can be configured to receive, position, and/or at least partially cover an electronic device including one or more sensors for measuring one or more physiological parameters of the subject. For example, the wearable device 3100 can be configured to receive, position, and/or at least partially cover electronic device 3200, electronic device 2700, electronic device 2700′, and/or any of the electronic devices described in U.S. Patent Pub. No. 2024/0081656 incorporated by reference herein. The wearable device 3100 can be configured to be secured to a subject and operably position an electronic device, such as electronic device 3200, electronic device 2700, electronic device 2700′, and/or any of the electronic devices described in U.S. Patent Pub. No. 2024/0081656 incorporated by reference herein. In some implementations, the wearable system 3000 includes the wearable device 3100 and the electronic device 3200. As shown in FIGS. 11A-11H, the wearable device 3100 and the electronic device 3200 can form a unitary structure configured to be secured to a subject (for example, a wrist of the subject).

FIGS. 11I-11J illustrate the electronic device 3200 disconnected from the wearable device 3100. Although the figures illustrate implementations of the wearable system 3000 in which the wearable device 3100 and the electronic device 3200 are removably connectable to one another, these components may be integrally formed with one another or configured to be non-separable after being secured together. For example, in some variants, the wearable device 3100 and electronic device 3200 are integrally formed.

Implementations of the wearable system 3000 in which electronic device 3200 is removably connectable from wearable device 3100 can confer a number of advantages. For example, implementations of the wearable system 3000 in which electronic device 3200 is removably connectable from wearable device 3100 can advantageously allow for a wearable device 3100 of various sizes (for example, small, medium, large, extra-large) and/or shapes to be utilized with the wearable system 3000, for example, so as to accommodate various sizes and/or shapes of a subject's body parts (for example, wrist). In this way, the wearable system 3000 can be customized to a subject by selecting an appropriately configured wearable device 3100 while allowing for other aspects of the wearable system 3000, such as the electronic device 3200, to remain the same and/or be universal across subjects. In another example, implementations of the wearable system 3000 in which electronic device 3200 is removably connectable from wearable device 3100 can advantageously allow for the wearable device 3100 to be a disposable component while the electronic device 3200 can be a reusable component. In some implementations, a securement portion (for example, a strap) of the wearable device 3100 can be customized and/or cut to length to accommodate various sizes and/or shapes of a subject's body parts (for example, wrist).

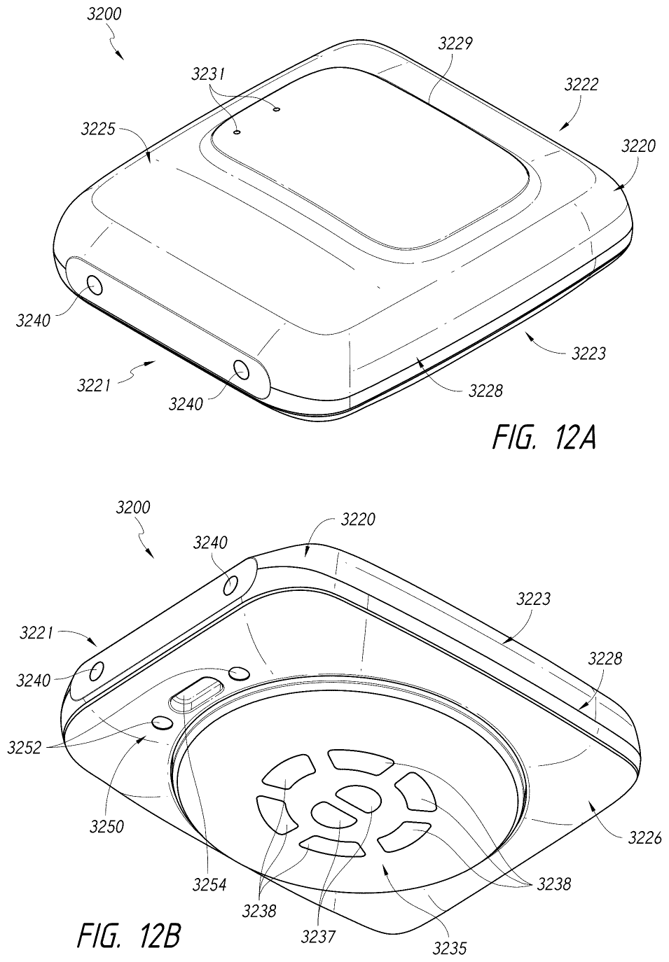

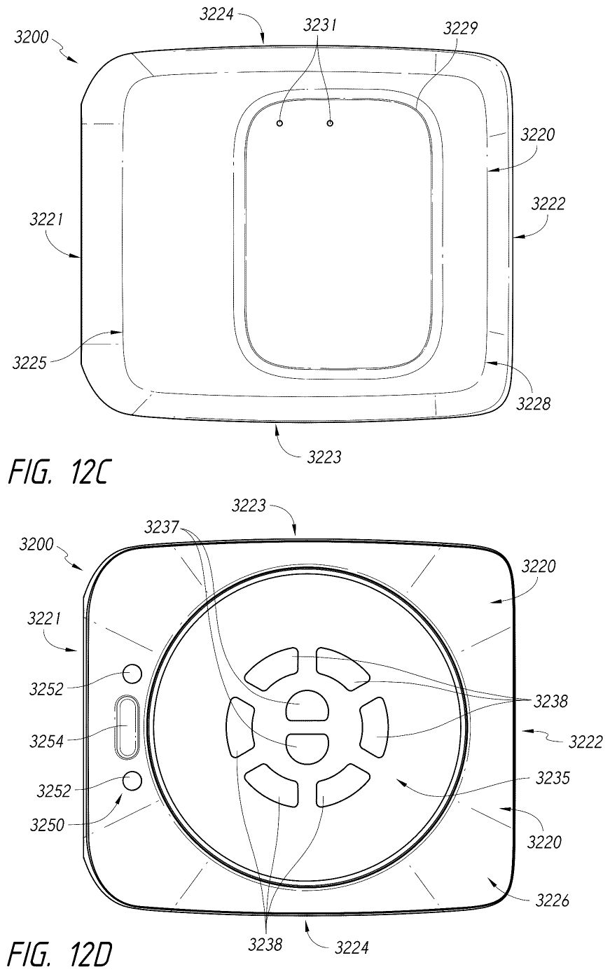

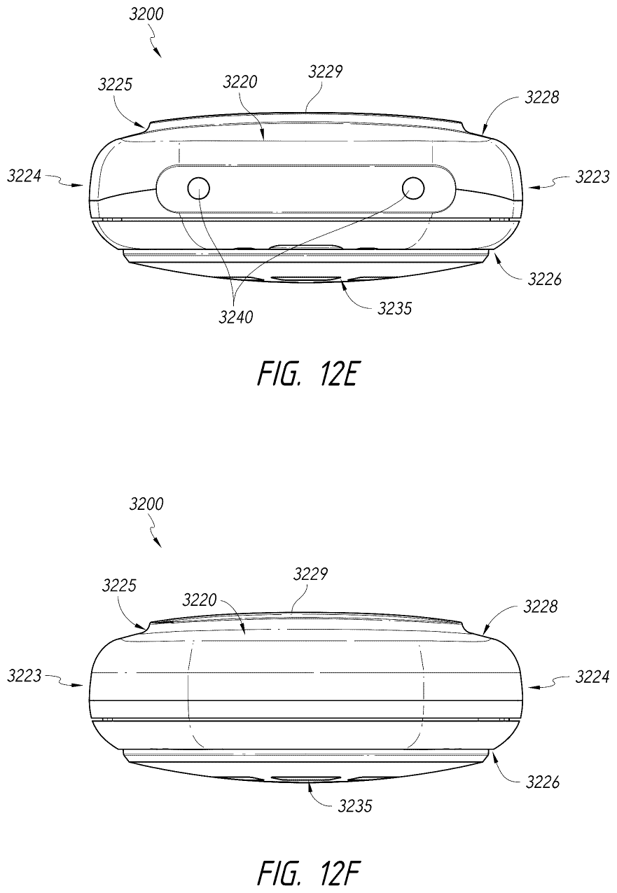

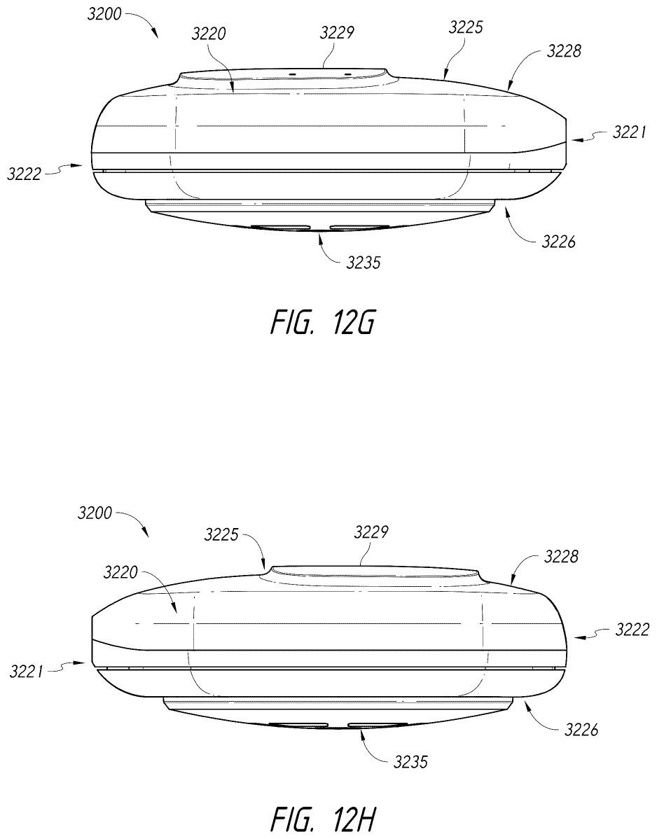

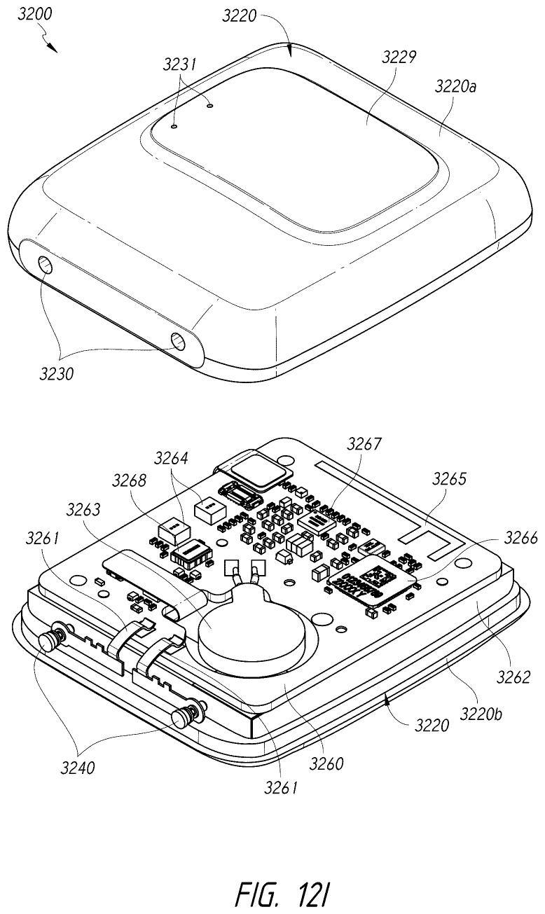

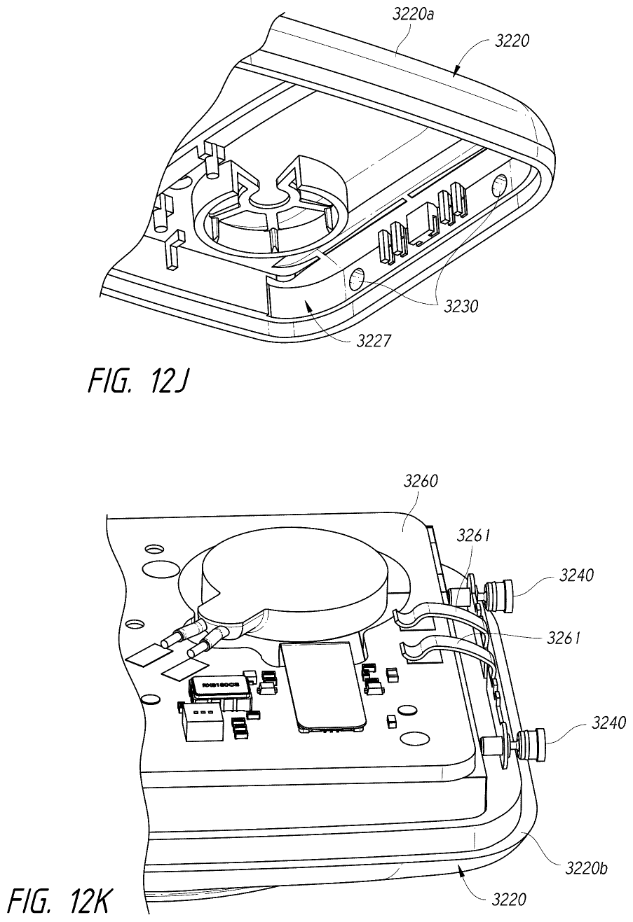

FIGS. 12A-12H illustrate various views of the electronic device 3200 that can be incorporated into any of the wearable systems described herein (for example, in wearable system 3000). FIG. 12A shows a top perspective view, FIG. 12B shows a bottom perspective view, FIG. 12C shows a top view, FIG. 12D shows a bottom view, FIG. 12E shows a front view, FIG. 12F shows a rear view, and FIGS. 12G-12H show side views, respectively, of the electronic device 3200. The electronic device 3200 can include any one or more or fewer of the features illustrated and discussed with respect to FIG. 10, including the one or more processors 3201, the one or more storage components 3202, the one or more communication components 3203, the battery 3204, the one or more status indicators 3206, the one or more emitters 3207, the one or more detectors 3208, the one or more ECG electrodes 3209, the one or more other sensors 3210, and/or the one or more other components 3211. Furthermore, the electronic device 3200 can be similar or identical to and/or incorporate any of the features described and/or illustrated with respect to electronic device 2700, electronic device 2700′, and/or with respect to any of the devices, assemblies, and/or systems described and/or illustrated in U.S. Patent Pub. No. 2024/0081656 incorporated by reference herein. The electronic device 2700 and/or the electronic device 2700′ described herein and/or any of the electronic devices described in U.S. Patent Pub. No. 2024/0081656 incorporated by reference herein can incorporate any of the features described and/or illustrated with respect to electronic device 3200.

As shown in FIGS. 12A-12H, electronic device 3200 can have a first end 3221, a second end 3222, a side 3233, a side 3224, a top 3225, and a bottom 3226. The second end 3222 can be opposite the first end 3221. The side 3243 can be opposite the side 3244. The top 3225 can be opposite the bottom 3226. The electronic device 3200 can have a housing 3220 that forms an exterior 3228 and an interior 3227 (shown in FIG. 12J) of the electronic device 3200. The housing 3220 can include a first portion 3220a (shown in FIGS. 12I-12K, which can also be referred to herein as a “top portion” or “top shell”) and a second portion 3220b (shown in FIGS. 12I-12K, which can also be referred to herein as a “bottom portion” or “bottom shell”). The first portion 3220a can be disposed at the top 3225, and the second portion 3220b can be disposed at the bottom 3226 of electronic device 3200.

As shown in the top perspective, bottom perspective, top, and bottom views of the electronic device 3200 in FIGS. 12A-12D, respectively, when viewed from the top or the bottom the electronic device 3200 can have a rounded rectangular shape. When viewed from its ends, such as shown in FIGS. 12E-12F, the sides 3223 and 3224 of electronic device 3200 can have a rounded shape (for example, convex rounded shape) extending from the top 3225 to the bottom 3226. When viewed from its sides, such as shown in FIGS. 12G-12H, the second end 3222 can have a rounded shape (for example, convex rounded shape) extending from the top 3225 to the bottom 3226. Additionally, the second end 3222 can taper inwards towards the top 3225 as shown, which can facilitate securement and removal of the electronic device 3200 with a wearable device as described herein (for example, wearable device 3100). Furthermore, and when viewed from its sides such as shown in FIGS. 12G-12H, the first end 3221 can have a planar (for example, flat) portion that extends at least partially between the top 3225 and the bottom 3226. Electrical contact(s) 3240 of electronic device 3200 can be positioned along such planar portion of first end 3221. Such planar portion of first end 3221 can advantageously facilitate contact of electrical contact(s) 3240 with corresponding electrical contacts of an associated wearable device (for example, with electrical contacts of wearable device 3100 described herein). The first end 3221 can have rounded portions adjacent such planar portion to transition between the top 3225 and the bottom 3226.

In some implementations and as shown in FIG. 12A, FIG. 12C, and FIGS. 12E-12H, the electronic device 3200 has a raised portion 3229. The raised portion 3229 can be on a portion of the exterior 3228 of housing 3220. For example, raised portion 3229 can be spaced from and/or raised with respect to a portion of exterior 3228 of housing 3220. The raised portion 3229 can be positioned at or along a portion of the top 3225. In some implementations and as shown, the raised portion 3229 is positioned asymmetrically about the exterior 3228 of housing 3220. Such asymmetric positioning of the raised portion 3229 can advantageously aid in the correct positioning of electronic device 3200 within an associated wearable device. For example, such asymmetric positioning of the raised portion 3229 can ensure the electronic device 3200 is secured in a specific orientation with respect to an associated wearable device. As another example, such asymmetric positioning of the raised portion 3229 can advantageously ensure electrical contact(s) 3240 of electronic device 3200 contact associated electrical contacts of a wearable device.

Electrical contact(s) 3240 of electronic device 3200 can be positioned at first end 3221. As shown, electronic device 3200 can include two electrical contact(s) 3240, however in some implementations the electronic device 3200 can include less than two or more than two electrical contact(s) 3240. Electrical contact(s) 3240 can sit flush with exterior 3228 or extend outward of or beyond exterior 3288. Furthermore and as shown, electrical contact(s) 3240 can be positioned along the planar portion of first end 3221. Electrical contact(s) 3240 can be rigid. In some implementations, electrical contact(s) 3240 can be resilient or configured to have a spring-like movement with respect to housing 3220 (for example, can move or compress inward with force and return outward upon removal of such force). Electrical contact(s) 3240 can extend through opening(s) 3230 (shown in FIG. 12I) of housing 3220.

Electronic device 3200 can include windows 3231 as part of status indicator(s) 3206. Such windows 3231 (which may also be referred to as “openings”) can be formed in a portion of housing 3220, such as through first portion 3220a as shown. Such positioning can enable a subject to view light emitted from the status indicator(s) 3206 when the wearable system 3000 is in use depending upon the wearable device utilized. In some implementations, such windows 3231 are formed through raised portion 3229 when included. The windows 3231 can be configured to allow light, such as light emitted from LEDs 3264 that form another part of status indicator(s) 3206 (shown in FIG. 12I) to pass through the housing 3220.

As shown in at least FIG. 12B and FIG. 12D, electronic device 3200 can include a pulse oximetry sensor 3235 (which can also be referred to herein as “sensor”). Such sensor 3235 can be an implementation of the pulse oximetry sensor described with respect to FIG. 10. Sensor 3235 can be positioned along bottom 3226 of electronic device 3200. Furthermore, sensor 3235 can include one or more emitters 3237 and one or more detectors 3238, which can be implementations of emitter(s) 3207 and detector(s) 3208 described with respect to FIG. 10. Although not shown, when ECG electrode(s) 3209 are included in electronic device 3200 they can be positioned adjacent sensor 3235 or integrated within sensor 3235. Such positioning of the one or more emitters 3237, the one or more detectors 3238, and the one or more ECG electrodes 3209 if included can facilitate physiological measurements of the subject when the electronic device 3200 is positioned by a wearable device as described herein. For example, the bottom 3226 can be positioned by a wearable device as described herein such that it is placed against a tissue site of the subject (for example, against skin of the subject). As shown in FIG. 12D, the one or more emitters 3237 can be positioned substantially about the center of the bottom 3226 of the electronic device 3200. Further as shown in FIG. 12D, the one or more detectors 3238 can be positioned around the one or more emitters 3237, for example, in a substantially annular/circular configuration that at least partially surrounds the one or more emitters 3237.

As described with respect to FIG. 10, the electronic device 3200 can be powered by a battery 3204, an implementation of which can be battery 3262 shown in FIG. 12I. The battery 3262 can be rechargeable. The battery 3262 can be charged/recharged by connecting the electronic device 3200 to a source of electrical power. For this, the electronic device 3200 can include a charge port 3250 configured to charge/recharge the battery 3262. The charge port 3250 can include electrical contacts 3252 and a feature 3254 that can be similar or identical to the charge port 2723 with electrical contacts 2724 and feature 2725 described with respect to electronic device 2700, to the charge port 1312 with electrical contacts 1313 and feature 1314 of the electronic device 1300 described in U.S. Patent Pub. No. 2024/0081656 incorporated by reference herein, and/or to the charge port 1212 with electrical contacts 1213 and feature 1214 of the electronic device 1200 described in U.S. Patent Pub. No. 2024/0081656 incorporated by reference herein. Such charge port 3250 can be disposed on the bottom 3226. Electrical contacts 3252 can electrically connect the battery 3262 to a source of power. Feature 3254 can be configured to aid in positioning corresponding electrical contacts of a charger (for example, charger 1500 illustrated and described with respect to FIGS. 5A-5C of U.S. Patent Pub. No. 2024/0081656 incorporated by reference herein) with electrical contacts 3252.

FIGS. 12I-12K illustrate partially exploded views of electronic device 3200. As shown, top portion 3220a of housing 3220 has been separated from and positioned above the remainder of electronic device 3200. With top portion 3220a separated, opening(s) 3230 in top portion 3220a for electrical contact(s) 3240 can be seen. Also with top portion 3220a separated, components of electronic device 3200 positioned within the electronic device 3200 (for example, within interior 3227 of housing 3220) can be seen. As shown, the electronic device 3200 can include a PCB 3260, a processor 3266, an inertial sensor 3267, an audio component 3268, a vibration motor 3263, an antenna 3265, the LEDs 3264, the battery 3262, and/or a flexible circuit 3261 within interior 3227. The processor 3266 can be an implementation of processor(s) 3201 described with respect to FIG. 10. The inertial sensor 3267 can be an implementation of other sensor(s) 3210 described with respect to FIG. 10. The audio component 3268 can be an implementation of other component(s) 3211 described with respect to FIG. 10. In some implementations, such audio component 3268 can be a buzzer that produces a narrow band or single wavelength of sound, or a speaker that can produce one or more wavelengths of sound. The vibration motor 3263 can be an implementation of other component(s) 3211 described with respect to FIG. 10. The antenna 3265 can be an implementation of communication component(s) 3203 described with respect to FIG. 10. Antenna 3265 can be configured to operate in any of the ways which are described above with respect to communication component 3203. In some implementations, such antenna 3265 is integrated with PCB 3260 as shown. In some implementations, antenna 3265 is configured similar to or the same as the antenna 2770 of electronic device 2700 and/or electronic device 2700′ described herein. The LEDs 3264 can be an implementation of status indicator(s) 3206 described with respect to FIG. 10. The flexible circuit 3261 can be configured to electrically connect electrical contacts 3240 with PCB 3260 and/or any components in electrical communication with PCB 3260. As shown, flexible circuit 3261 can have an arched shape configured to confer electrical contacts 3240 with resilience. Further as shown, flexible circuit 3261 can have a first portion that connects to a first electrical contact 3240 and a second portion that connects to a second electrical contact 3240, which can advantageously provide each of the first and second electrical contacts 3240 with independent resiliency and/or movement for maximizing contact of such first and second electrical contacts 3240 with electrical contacts of a wearable device as described herein.

FIGS. 13A-13H illustrate various views of the wearable device 3100 that can be incorporated into any of the wearable systems described herein (for example, in wearable system 3000). FIGS. 13A-13B show top perspective views, FIGS. 13C-13D show bottom perspective views, FIG. 13E shows a top view, FIG. 13F shows a bottom view, and FIGS. 13G-13H show side views, respectively, of the wearable device 3100. Wearable device 3100 can include any one or more or fewer of the features illustrated and discussed with respect to FIG. 10, including the one or more storage components 3102 and/or the one or more communication components 3103. Furthermore, the wearable device 3100 can be similar or identical to and/or incorporate any of the features described and/or illustrated with respect to any of the devices, assemblies, and/or systems described and/or illustrated in U.S. Patent Pub. No. 2024/0081656 incorporated by reference herein. Any of the wearable devices described in U.S. Patent Pub. No. 2024/0081656 incorporated by reference herein can incorporate any of the features described and/or illustrated with respect to wearable device 3100.

Wearable device 3100 can be configured to secure around a portion of a subject's body (for example, a wrist). Wearable device 3100 can include a main body 3105 (which can also be referred to herein as “body portion”) and a securement portion connected to the main body 3105. Such securement portion can be a strap 3180 as shown. Main body 3105 can have a top 3115, a bottom 3117 opposite the top 3115, a first end 3111, a second end 2112 opposite the first end 3111, a side 3113, and a side 3114 opposite the side 3113. The strap 3180 can connect to the main body 3105 at first end 3111 and at second end 3112. For example and as shown, strap 3180 can connect to main body 3105 at first end 3111 via structure 3123 at first end 3111 and at second end 3112 via structure 3124 at second end 3112. For this, structures 3123, 3124 can be configured to connect to and/or receive at least a portion of strap 3180. For example, structures 3123, 3124 can be a slot in main body 3105 and a bar extending adjacent to and enclosing the slot. In some implementations, strap 3180 is configured to connect to main body 3105 via structures 3123, 3124 and secure to itself. In some implementations, strap 3180 or at least an end thereof can be integrally formed with main body 3105.

Main body 3105 can include a frame 3110. Frame 3110 can include any of the features described with respect to main body 3105 and vice versa. Main body 3105 can include a cavity 3120 configured to at least partially receive and position an electronic device as described herein, for example electronic device 3200. Such cavity 3120 can be accessible from bottom 3117 of main body 3105. The bottom 3117 can be configured to face toward a tissue site of the subject (for example, toward skin of the subject) when the wearable device 3100 is secured to the subject. Such configuration can allow the one or more emitters 3237, the one or more detectors 3238, and/or the one or more ECG electrodes 3209 when included of the electronic device 3200 to contact and/or face toward the tissue site of the subject (for example, toward skin of the subject) via the cavity 3120 (for example, by being positioned at least partially within cavity 3120). For example, FIGS. 11B and FIGS. 11E-11F illustrate the electronic device 3200 positioned by the wearable device 3100 such that the one or more emitters 3237 and the one or more detectors 3238 of electronic device 3200 are accessible to contact and/or face toward the tissue site of the subject via the cavity 3120.

In some implementations, main body 3105 includes an opening 3116 in top 3115. Such opening 3116 can be configured to at least partially receive and/or position raised portion 3229 of electronic device 3200, for example as shown in FIG. 11A, FIG. 11C and FIGS. 11E-11F, when the electronic device 3200 and wearable device 3100 are secured to one another. In some implementations, opening 3116 receives raised portion 3229 when the electronic device 3200 and wearable device 3100 are secured to one another such that the raised portion 3229 sits substantially flush with top 3115 of main body 3105. In some implementations, raised portion 3229 extends through opening 3116 when the electronic device 3200 and wearable device 3100 are secured to one another. Further as shown, in some implementations opening 3116 is positioned asymmetrically with respect to the first end 3111 and second end 3112, such as closer to second end 3112 than first end 3111. Such asymmetric positioning can advantageously ensure that electronic device 3200 is secured in a specific position (for example, a single position) with respect to wearable device 3100. Furthermore, such asymmetric positioning can advantageously position electronic device 3200 with respect to wearable device 3100 such that electrical contacts 3240 contact electrical contacts (for example, flexible circuit 3160) of wearable device 3100.

Main body 3105 can be made of a resilient material configured to allow a size of the cavity 3120 to be increased to allow the electronic device 3200 to be at least partially inserted within the cavity 3120 and at least partially positioned/secured within the cavity 3120. Furthermore, main body 3105 can be made of a resilient material configured to deform and/or flex when securing to the electronic device 3200. In some implementations, main body 3105 comprises a rigid and resilient material. In some implementations, main body 3105 comprises a flexible and/or stretchy and resilient material. Main body 3105 can advantageously allow the electronic device 3200 to be held securely by the main body 3105 and aid in preventing the electronic device 3200 from slipping or moving along a tissue site of the subject.

In some implementations, main body 3105 includes engagement features to retain and/or position the electronic device 3200 within the wearable device 3100 (for example, at least partially within cavity 3120). As shown in at least FIG. 13C, main body 3105 can include a retention mechanism 3150. Retention mechanism 3150 can comprise a ridge 3151 (which can also be referred to herein as a “tapered portion”). Ridge 3151 be positioned at or adjacent first end 3111. Ridge 3151 can extend along first end 3111 between (for example, at least partially between) side 3113 and side 3114. Retention mechanism 3150 can advantageously guide and/or position end 3221 of electronic device 3200 such that, when electronic device 3200 is secured by wearable device 3100, electrical contacts 3240 of electronic device 3200 contact electrical contacts of wearable device 3100 (for example, flexible circuit 3160). Furthermore, and as shown in at least FIG. 13D, main body 3105 can include one or more ridge(s) 3119. Ridge(s) 3119 can be positioned at or adjacent second end 3112. Ridge(s) 3119 can extend along second end 3112 between (for example, at least partially between) side 3113 and side 3114. As shown in at least FIGS. 13C-13D, retention mechanism 3150, which can include ridge 3151, and ridge(s) 3119 can be positioned adjacent cavity 3120 at bottom 3117 of main body 3105. Retention mechanism 3150, which can include ridge 3151, and ridge(s) 3119 can allow the electronic device 3200 to be removably received by the main body 3105 and secure the electronic device 3200 within the main body 3105 (for example, within cavity 3120) when the electronic device 3200 and wearable device 3100 are secured to one another. In some implementations, the releasable connection between the wearable device 3100 and the electronic device 3200 is a snap fit.

In some implementations and as shown, main body 3105 of wearable device 3100 can have a width (for example, extending between side 3113 and side 3114) that is greater than a width of the securement portion (for example, a width of strap 3108). In variants of the wearable device 3100, main body 3105 can have a width (for example, extending between side 3113 and side 3114) that is substantially similar or the same as a width of the securement portion (for example, a width of strap 3108).

FIG. 13I illustrates various implementations of strap 3180 of wearable device 3100. Strap 3180 can be made of a flexible, stretchy, and/or resilient material. Such material of the strap 3180 can advantageously allow the strap 3180 to secure the main body 3105 to the subject and prevent the electronic device 3200 held by the main body 3105 from slipping, moving, and/or coming away from the tissue site of the subject (e.g., the skin of the subject). Strap 3180 can have a uniform width or a variable width. Strap 3180 can display at least a portion of patient identification data 3186 of the patient as described herein, such as shown in FIG. 13I. Such patient identification data 3186 can be printed on strap 3180 in the healthcare environment. In some implementations, strap 3180 is connected to main body 3105 in the healthcare environment. Strap 3180 can be configured for single use. In some implementations, strap 3180 is configured to be cut to size for the patient. Strap 3180 can be non-removable once attached to the patient, requiring the strap 3180 to be cut in order to be removed. Strap 3180 can also include tamper detections and alarms which indicate strap 3180 has been removed improperly or tampered with. In some implementations, strap 3180 can include an RFID (radio frequency identification) tag that can, for example, store at least a portion of patient identification data 3186. In some implementations, such an RFID tag can allow the patient to get through security checkpoints and/or various areas of a healthcare facility. Optionally, strap 3180 can include a location determination device that can determine exact or approximate locations of a patient.

FIGS. 14A-14F illustrate various views of main body 3105 of wearable device 3100. FIGS. 14A-14B show bottom perspective views of main body 3105 where cavity 3120, retention mechanism 3150 comprising ridge 3151, ridge(s) 3119, and flexible circuit 3160, when such features are included, can be more easily seen. FIGS. 14C-14F show various views of an implementation of wearable device 3100 wherein main body 3105 includes a slot 3121 adjacent cavity 3120 and a support structure 3130 for supporting and/or operably positioning the flexible circuit 3160 and a storage component 3170. Slot 3121 can be positioned at or adjacent the first end 3111 of main body 3105. Slot 3121 can be configured to receive and position at least the support structure 3130 and flexible circuit 3160. FIG. 14C is an exploded view showing such support structure 3130 and flexible circuit 3160 removed from slot 3121, and FIG. 14D shows main body 3105 with slot 3121 without support structure 3130 and flexible circuit 3160.

FIGS. 14E-14F show exploded views of the support structure 3130, flexible circuit 3160, and a tape structure 3140 while removed from slot 3121 of main body 3105. As shown, support structure 3130 can include a cavity 3136 (which can also be referred to as a “chip cavity”), openings 3132, fingers 3134, and/or recesses 3138. In some implementations and as shown, the retention mechanism 3150 including ridge 3151 can be integrally formed with support structure 3130. Also shown, the flexible circuit 3160 can include a base 3162, arms 3164, and/or ends 3165. Tape structure 3140 can be configured to secure flexible circuit 3160 to support structure 3130. Tape structure 3140 can include openings 3142 to allow portions of flexible circuit 3160 to be accessible. For example, arms 3164 or portions thereof can be accessible via openings 3142 and/or extend at least partially through openings 3142 of tape structure 3140 when tape structure 3140, flexible circuit 3160, and support structure 3130 are joined together.

The cavity 3136 of support structure 3130 can be configured to position and/or receive storage component 3170. For this, the cavity 3136 can be sized and/or shaped to receive storage component 3170 at least partially therein. Fingers 3134 of support structure 3130 can be configured to support and/or bias portions of the flexible circuit 3160. For example and as shown, at least a portion of fingers 3134 can have an arcuate shape configured to bias portions of flexible circuit 3160 (such as arms 3164 of flexible circuit 3160) towards cavity 3120. Such biasing can aid in electrical coupling of the flexible circuit 3160 with electrical contacts 3240 of electronic device 3200, for example. Fingers 3134 can extend at least partially across openings 3132 in a cantilever fashion. Recesses 3138 can be configured to position and/or receive ends 3165 of flexible circuit 3160. For this, recesses 3138 can be sized and/or shaped to receive ends 3165 at least partially therein. As shown, support structure 3130 can be symmetric about cavity 3136 and comprise two fingers 3134, two openings 3132, and two recesses 3138.

Flexible circuit 3160 can be configured to electrically connect storage component 3170 with electronic device 3200 when electronic device 3200 and wearable device 3100 are secured to one another (for example, when electronic device 3200 is secured within cavity 3120). For this, storage component 3170 can be attached and/or connected to flexible circuit 3160. Storage component 3170 can be mounted to base 3162 as shown (for example, in a direction facing cavity 3136 of support structure 3130). Storage component 3170 (which can also be referred to as a “chip” or “memory chip”) can be an implementation of storage component(s) 3102 described with respect to FIG. 10. Arms 3164 can extend from base 3162 and comprise electrical contacts of the flexible circuit 3160. As shown, flexible circuit 3160 can include two arms 3164 that extend from base 3162 in generally opposite directions. Ends 3165 can be positioned at the end of arms 3164 and can be configured to aid in connecting flexible circuit 3160 with support structure 3130. For example and as shown, flexible circuit 3160 can have T-shaped ends 3165 that fit into recesses 3138 of support structure 3130. Arms 3164 can be at least partially arcuate and can be supported by the fingers 3134 of support structure 3130 when flexible circuit 3160 and support structure 3130 are joined together (for example, joined together by tape structure 3140). Such arcuate portions of arms 3164 can comprise the electrical contacts of flexible circuit 3160.

When electronic device 3200 is inserted into cavity 3120 of wearable device 3100, electrical contacts 3240 can contact flexible circuit 3160. For example, arms 3164 comprising electrical contacts of flexible circuit 3160, which can be biased towards cavity 3120 by fingers 3134 of support structure 3130, can contact electrical contacts 3240 of electronic device 3200 when the devices are secured to one another. Upon such securement, arms 3164 and fingers 3134 can be forced away from cavity 3120 by electrical contacts 3240. Thus, arms 3164 and fingers 3134 can have a first position when electronic device 3200 is not secured by wearable device 3100, and a second position when electronic device 3200 is secured by wearable device 3100.

To secure electronic device 3200 to wearable device 3100, first end 3221 of electronic device can be inserted into cavity 3120 at first end 3111 of main body 3105. Such insertion can be performed with electronic device 3200 at an angle with respect to main body 3105. Retention mechanism 3150, which can comprise ridge 3151, can guide first end 3221 of electronic device into cavity 3120 such that electrical contacts 3240 at least partially contact flexible circuit 3160 (for example, contact arms 3164 comprising electrical contacts of flexible circuit 3160). Second end 3222 of electronic device 3200 can then be pushed towards main body 3105 to completely secure electronic device 3200 with main body 3105. Ridges 3119 at second end 3112 of main body 3105 can aid in securing second end 3222 within cavity 3120. When electronic device is fully secured by main body 3105, electrical contacts 3240 of electronic device 3200 can contact flexible circuit 3160 as described herein.

In some implementations, wearable device 3100 does not include communication components such as communication component(s) 3103 described with respect to FIG. 10. Instead, communication of wearable device 3100 with an electronic device, such as electronic device 3200, occurs via physical contact, such as contact between electrical contacts 3240 and flexible circuit 3160 as described herein.

In a variant of main body 3105, tape structure 3140 is excluded and flexible circuit 3160 is positioned between support structure 3130 and first end 3111. In such variant, portions of flexible circuit equivalent to arms 3164 can extend through openings in support structure 3130 and towards cavity 3120 to allow for an electrical connection between flexible circuit 3160 and electronic device 3200.

In another variant of main body 3105, main body 3105 does not comprise exposed electrical contacts such as flexible circuit 3160. In such variant the wearable device 3100 can include the storage component(s) 3102 and communication component(s) 3103. In such variant communication between wearable device 3100 and an electronic device can occur wirelessly. Furthermore, in such variant opening 3116 can be omitted, for example, since a specific positioning of an electronic device with the wearable device of this variant may not be required.

FIG. 15 is a flowchart illustrating an example method 4000 of data transmission between an electronic device, a wearable device, and an external device as described herein. Method 4000 can be performed, for example, by electronic device 3200, wearable device 3100, and any of the monitoring hubs or components thereof described herein, or by any of the electronic devices and wearable devices described herein and/or in U.S. Patent Pub. No. 2024/0081656 incorporated by reference herein. One or more hardware processors of an electronic device as described herein can execute method 4000, or portions thereof. Method 4000 is provided as an example and is not intended to be limiting of the present disclosure. In some implementations, one or more hardware processors executing the method 4000 may omit portions of the method 4000, may add additional operations, and/or may rearrange an order in which the operations of the method 4000 are executed.

At block 4010, an electronic device is obtained. The electronic device can be any of the electronic devices described herein, such as electronic device 2700, 2700′, or 3200, or any of the electronic devices described in U.S. Patent Pub. No. 2024/0081656 incorporated by reference herein.

At block 4020, a wearable device is obtained. The wearable device can be any of the wearable devices described herein, such as wearable device 3100, or any of the wearable devices described in U.S. Patent Pub. No. 2024/0081656 incorporated by reference herein.

At block 4030, patient identification data from the wearable device can be transmitted to the electronic device and/or the electronic device can receive patient identification data from the wearable device. As an example, patient identification data from wearable device 3100 can be transmitted to electronic device 3200. Such patient identification data can be patient identification data 3186 described herein, which can include one or more of a name, assigned identification number or health record number, date of birth, phone number, social security number, address, photo, dates of hospitalizations or encounters, name of attending physicians or care providers, demographics, diagnoses, problems list, progress notes, medications, vital signs, laboratory data, tests, allergies, immunizations, treatment plans, tag, marker, serial number, bar code, QR code, facial recognition, fingerprint recognition, voice recognition, eye recognition, gesture recognition, biometric data, data from the patient's electronic health record, or the like, which may be unique to the patient. Such patient identification data can be stored in storage component(s) 3102, an implementation of which can be storage component 3170 of wearable device 3100. In some implementations, such patient identification data can be transmitted, written, and/or stored to the wearable device in a healthcare environment. For example, patient identification data 3186 can be transmitted, written, and/or stored to storage component 3170 of wearable device 3100 in the healthcare environment.

In some implementations, patient identification data is transmitted from the wearable device to the electronic device automatically, for example, when the electronic device is secured by the wearable device. For example, patient identification data 3186 can be transmitted from wearable device 3100 to electronic device 3200 automatically when electrical contacts 3240 of electronic device 3200 contact electrical contacts 3164 of wearable device 3100. In some implementations, such automatic transmission of patient identification data can occur when the electronic device is within proximity of the wearable device. For example, and in variants of a wearable device and an electronic device that do not have electrical contacts, such transmission of patient identification data can occur wirelessly via communication components of each. Hardware processor(s) of electronic device can control and/or implement such transmission.

The electronic device can be secured by the wearable device and the wearable device can be secured to a patient. Upon being secured to the patient, the electronic device can measure one or more physiological parameters of the patient as described herein. Physiological data associated with such one or more physiological parameters of the patient can be associated with the patient identification data. Hardware processor(s) of the electronic device can control and/or implement such association. In other words, physiological data associated with the one or more physiological parameters measured by the electronic device can be linked to the patient via the patient identification data the electronic device receives from the wearable device.

At block 4040, the physiological data associated with the patient identification data can be transmitted (for example, wirelessly) to an external device. For example, electronic device 3200 can transmit physiological data based on one or more physiological parameters it measures of the patient that is associated with the patient identification data 3186 to an external device. Such an external device can be a monitoring hub or a portion thereof or any of the external devices described herein. Hardware processor(s) of the electronic device can control and/or implement such transmission. In some implementations, patient identification data can be transmitted to the external device without associated physiological data. The electronic device can request to establish a wireless communication connection with the external device. Such request can be performed prior to or concurrent with the transmission of physiological data and/or patient identification data. Pursuant to establishing the wireless communication connection with the external device, the electronic device can provide real-time physiological data to the external device (for example, physiological data measured by the one or more physiological sensors thereof).

In some implementations, an external device (for example, a monitoring hub or portion thereof as described herein) can access patient identification information from a wearable system (for example, from an electronic device or wearable device). The external device can provide the patient identification information to a server to retrieve historical physiological data from the server. Such a server can be a remote server. Furthermore, such patient identification information can be useable to identify the historical physiological data and verify permission to access the historical physiological data. The external device can access, from the server, the historical physiological data. Such actions by the external device can be performed and/or directed by hardware processor(s) of the external device. The historical physiological data can be associated with the subject and originate from an at-home monitoring device (for example, from an at-home monitoring device before the patient enters a healthcare environment). The historical physiological data can additionally or alternatively be associated with the subject and originate from a wearable system used previously in the healthcare environment.

In some implementations, the external device is configured to wirelessly receive real-time physiological data from the electronic device. The external device can generate one or more physiological parameters from the real-time physiological data. In some cases, the external device can generate one or more physiological parameters from the real-time physiological data and historical physiological data. The external device can display indicia of the one or more physiological parameters via a display thereof when included. Such actions by the external device can be performed and/or directed by hardware processor(s) of the external device.

In some implementations, the electronic device is configured to determine if the wearable device is an authorized product. Hardware processor(s) of the electronic device can control and/or implement such determination. The electronic device can perform such determination upon initial communication with the wearable device, such as when the electronic device is connected electronically or wirelessly thereto. Furthermore, the electronic device can perform such determination prior to measuring the one or more physiological parameters of the patient, and/or prior to the wearable system being secured to the patient. The storage component of the wearable device can store product information used by the electronic device to determine if it is an authorized product.

In some implementations, the electronic device is configured to transmit operational data to the wearable device, such as to the storage component of the wearable device. Hardware processor(s) of the electronic device can control and/or implement such transmission. Such operational data can include a duration of time the electronic device, the wearable device, and/or the wearable system is in use, for example, a duration of time that the electronic device is measuring one or more physiological parameters of the patient (which can correspond to a duration of time the wearable device is secured to the patient and a duration of time the wearable system is in use). The wearable device can be configured to become non-operational when the duration of time the wearable system is in use reaches a threshold value. In some implementations, the storage component of the wearable device can include a counter that is updated by the electronic device corresponding to a duration of time the electronic device and/or the wearable device is in use. Further to such implementations, the wearable device can be made non-operational and/or configured to not be usable after being separated from the electronic device upon reaching a threshold value of such counter. In some implementations, the wearable device can be configured to be non-operational after a day, days, a week, weeks, a month, months, and/or after being separated from an electronic device.

FIG. 16 is a flowchart illustrating an example method 5000 of data transmission between an electronic device, a wearable device, and an external device as described herein. Method 5000 can be performed, for example, by electronic device 3200, wearable device 3100, and any of the monitoring hubs or components thereof described herein, or by any of the electronic devices and wearable devices described herein and/or in U.S. Patent Pub. No. 2024/0081656 incorporated by reference herein. One or more hardware processors of an electronic device as described herein can execute method 5000, or portions thereof. Method 5000 is provided as an example and is not intended to be limiting of the present disclosure. In some implementations, one or more hardware processors executing the method 5000 may omit portions of the method 5000, may add additional operations, and/or may rearrange an order in which the operations of the method 5000 are executed. In some implementations, method 5000 may include any one or more of the operations described with respect to method 4000.

At block 5010, an electronic device is obtained. Block 5010 can be the same as or similar to and include any one or more aspects as block 4010 described with respect to method 4000 of FIG. 15. The electronic device can be any of the electronic devices described herein, such as electronic device 2700, 2700′ or 3200, or any of the electronic devices described in U.S. Patent Pub. No. 2024/0081656 incorporated by reference herein.

At block 5020, a wearable device is obtained. Block 5020 can be the same as or similar to and include any one or more aspects as block 4020 described with respect to method 4000 of FIG. 15. The wearable device can be any of the wearable devices described herein, such as wearable device 3100, or any of the wearable devices described in U.S. Patent Pub. No. 2024/0081656 incorporated by reference herein.

At block 5030, patient identification data from the wearable device can be transmitted to the electronic device and/or the electronic device can receive patient identification data from the wearable device. Block 5030 can be the same as or similar to and include any one or more aspects as block 4030 described with respect to method 4000 of FIG. 15. As an example, patient identification data from wearable device 3100 can be transmitted to electronic device 3200. Such patient identification data can be patient identification data 3186 described herein. Such patient identification data can be stored in storage component(s) 3102, an implementation of which can be storage component 3170 of wearable device 3100. In some implementations, such patient identification data can be transmitted, written, and/or stored to the wearable device in a healthcare environment. For example, patient identification data 3186 can be transmitted, written, and/or stored to storage component 3170 of wearable device 3100 in the healthcare environment.

At block 5040, historical physiological data can be transmitted to the electronic device and/or the electronic device can receive historical physiological data. In some implementations, such historical data is received (for example, wirelessly) from an at-home monitoring device. Further to such implementations, such historical data can be generated by the at-home monitoring device before the patient enters a healthcare environment. The electronic device can apply weights to the historical physiological data based on whether the historical physiological data originates from a medically approved device or an unapproved device and generate one or more physiological parameters from the historical physiological data with the weights. In some implementations, such historical data is received from a server (for example, a remote server). Hardware processor(s) of the electronic device can control such activities and communication.

At block 5050, the historical physiological data and the real-time physiological data associated with the patient identification data can be transmitted to the external device. The electronic device can transmit to the external device the historical physiological data and the real-time physiological data associated with the patient identification data. In some implementations, the electronic device provides the historical physiological data to the external device responsive to determining that the at-home monitoring device is a medically approved device. In some implementations, the electronic device, responsive to determining that the at-home monitoring device is an unapproved device, indicates that the historical physiological data originates from an unapproved device with metadata of the historical physiological data.

FIG. 17 is a flowchart illustrating an example method 6000 of data transmission between an electronic device, a wearable device, and an external device as described herein. Method 6000 can be performed, for example, by electronic device 3200, wearable device 3100, and any of the monitoring hubs or components thereof described herein, or by any of the electronic devices and wearable devices described herein and/or in U.S. Patent Pub. No. 2024/0081656 incorporated by reference herein. One or more hardware processors of an electronic device as described herein can execute method 6000, or portions thereof. Method 6000 is provided as an example and is not intended to be limiting of the present disclosure. In some implementations, one or more hardware processors executing the method 6000 may omit portions of the method 6000, may add additional operations, and/or may rearrange an order in which the operations of the method 6000 are executed. In some implementations, method 6000 may include any one or more of the operations described with respect to method 4000 and/or method 5000.

At block 6010, an electronic device is obtained and used to measure physiological data of a patient prior to entering a healthcare environment to generate historical physiological data. Such an electronic device can be any of the electronic devices described herein, such as electronic device 2700, 2700′, or 3200, or any of the electronic devices described in U.S. Patent Pub. No. 2024/0081656 incorporated by reference herein. Furthermore, such an electronic device can be secured by a wearable device as described herein or in U.S. Patent Pub. No. 2024/0081656 incorporated by reference herein and secured to the patient to measure the physiological data of the patient prior to entering the healthcare environment to generate the historical physiological data.

At block 6020, a wearable device is obtained. Such a wearable device can be any of the wearable devices described herein, such as wearable device 3100, or any of the wearable devices described in U.S. Patent Pub. No. 2024/0081656 incorporated by reference herein. Furthermore, such a wearable device can be obtained in the healthcare environment. The wearable device (for example, the storage component of the wearable device) can receive patient identification data associated with the patient in the healthcare environment as described herein. The electronic device can be coupled to (secured to) the wearable device in the healthcare environment. Such electronic device can be the same electronic device that was used to measure physiological data of the patient prior to entering the healthcare environment.

At block 6030, patient identification data from the wearable device can be transmitted to the electronic device and/or the electronic device can receive patient identification data from the wearable device. Block 6030 can be the same as or similar to and include any one or more aspects as block 4030 and/or block 5030 described with respect to methods 4000 and 5000, respectively. As an example, patient identification data from wearable device 3100 can be transmitted to electronic device 3200. Such patient identification data can be patient identification data 3186 described herein. Such patient identification data can be stored in storage component(s) 3102, an implementation of which can be storage component 3170 of wearable device 3100. The electronic device can measure real-time physiological data upon being secured to the wearable device and the wearable device being secured to the patient (when the wearable system comprising the electronic device and the wearable device is secured to the patient). Furthermore, the electronic device can associate the historical physiological data and the real-time physiological data with the patient identification data.

At block 6040, the historical physiological data and the real-time physiological data associated with the patient identification data can be transmitted to the external device. The electronic device can transmit to the external device the historical physiological data and the real-time physiological data associated with the patient identification data. Block 6040 can be the same as or similar to and include any one or more aspects as block 5050 described with respect to method 5000 of FIG. 16.

Communication between a wearable device, an electronic device, and an external device as described in methods 4000, 5000, and/or 6000 can be performed via any of the communication protocols described herein. In some implementations, such communication is performed wirelessly. In some implementations, such communication is performed via electrical contact between components as described herein.

### ADDITIONAL CONSIDERATIONS

The wearable devices described herein can include features and/or materials to enhance a grip of the wearable device onto skin of the patient to prevent an electronic device secured thereby from slipping or moving along the tissue site of the Patient. Such features can include bumps, a roughened surface texture, and the like. Such materials can include a tacky and/or rubber-like material (e.g., a silicone and/or silicone rubber), an adhesive material, or the like. In some implementations, such features can comprise such materials. Furthermore, such features and/or materials can be disposed at least partially along a body-contacting side of the wearable device (for example, the bottom of the wearable device). The wearable devices and/or the electronic devices described herein can include features and/or materials to enhance comfort when worn by a user.

The wearable systems described herein and any variations thereof and/or any of their components can be configured to be waterproof, water resistant, drip proof, shock proof, dust proof, and/or dust resistant. While the wearable systems have been described as having a rechargeable battery, the battery can be nonrechargeable or single use. In some implementations, a battery of the wearable system can be rechargeable but non-removable from the system. In such a case, the wearable system can include a charge port configured to receive a power cable for charging. In some variants, an electronic device of any of the wearable systems described herein can be permanently connected to a wearable device of any of the wearable systems described herein.

Some implementations of the wearable systems disclosed herein can advantageously provide for a wearable system that is reusable and/or durable and/or have components that are reusable and/or durable (for example, lasting weeks, months, and/or years). Any or all such components can be configured to be sanitized between uses and/or between subjects. Some implementations of the wearable systems disclosed herein can provide for an electronic device that is reusable and/or durable. Additionally, some implementations of the wearable systems disclosed herein can provide for a wearable device that is reusable and/or durable. Alternatively, some implementations of the wearable systems disclosed herein can provide for a wearable device that is disposable. In some implementations, reusable components of the wearable systems described herein can be refurbished, reused, and/or reprocessed. Such reusable components can be combined with other new, refurbished, reused, and/or reprocessed components to form a portion or an entirety of a wearable system. In some implementations, such reusable components can be combined with disposable components to form a portion or an entirety of a wearable system. Some implementations of the disclosed wearable systems (or portions of such systems) can be disposable, which can reduce the risk of cross-contamination between multiple users.

Any of the features and/or functionality of the wearable systems and their components described herein can be implemented in another of the wearable systems described herein and/or in U.S. Patent Pub. No. 2024/0081656 incorporated by reference herein. For example, any of the wearable devices described herein can include any of the body portion configurations and/or securement portion configurations of another wearable device described herein and/or in U.S. Patent Pub. No. 2024/0081656 incorporated by reference herein. As another example, any of the wearable systems can be adapted to receive any one of the electronic devices described herein and/or in U.S. Patent Pub. No. 2024/0081656 incorporated by reference herein. Furthermore, any of the features and/or functionality of the wearable systems and their components described herein can be omitted in another of the wearable systems described herein.

Certain categories of persons, such as caregivers, clinicians, doctors, nurses, and friends and family of a user, may be used interchangeably to describe a person providing care to the user. Furthermore, patients or users used herein interchangeably refer to a person who is wearing a sensor or is connected to a sensor or whose measurements are used to determine a physiological parameter or a condition. Parameters may be, be associated with, and/or be represented by, measured values, display icons, alphanumeric characters, graphs, gages, power bars, trends, or combinations. Real time data may correspond to active monitoring of a user, however, such real time data may not be synchronous to an actual physiological state at a particular moment. Measurement value(s) of a parameter and the parameter used herein such as, SpO₂, RR, PaO₂ and the like, unless specifically stated otherwise, or otherwise understood with the context as used is generally intended to convey a measurement or determination that is responsive to the physiological parameter.

Although certain implementations and examples have been described herein, it will be understood by those skilled in the art that many aspects of the systems and devices shown and described in the present disclosure may be differently combined and/or modified to form still further implementations or acceptable examples. All such modifications and variations are intended to be included herein within the scope of this disclosure. A wide variety of designs and approaches are possible. No feature, structure, or step disclosed herein is essential or indispensable. The various features and methods described herein may be used independently of one another, or may be combined in various ways. For example, elements may be added to, removed from, or rearranged compared to the disclosed example implementations. All possible combinations and sub-combinations are intended to fall within the scope of this disclosure.

Any methods and processes described herein are not limited to any particular sequence, and the blocks or states relating thereto can be performed in other sequences that are appropriate. For example, described blocks or states may be performed in an order other than that specifically disclosed, or multiple blocks or states may be combined in a single block or state, or certain method or process blocks may be omitted, or certain blocks or states may be performed in a reverse order from what is shown and/or described. The example blocks or states may be performed in serial, in parallel, or in some other manner. Blocks or states may be added to or removed from the disclosed example implementations.

The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct entities or other users. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames).

Many other variations than those described herein will be apparent from this disclosure. For example, depending on the implementation, certain acts, events, or functions of any of the algorithms described herein can be performed in a different sequence, can be added, merged, or left out altogether (for example, not all described acts or events are necessary for the practice of the algorithms). Moreover, in certain implementations, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

Various illustrative logical blocks, modules, routines, and algorithm steps that may be described in connection with the disclosure herein can be implemented as electronic hardware (for example, ASICs or FPGA devices), computer software that runs on computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the implementations disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, controller, microcontroller, or state machine, combinations of the same, or the like. A processor can include electrical circuitry configured to process computer-executable instructions. A processor can include an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “could,” “might,” “may,” “for example,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”. “about”. “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

As used herein. “real-time” or “substantial real-time” may refer to events (for example, receiving, processing, transmitting, displaying etc.) that occur at a same time as each other, during a same time as each other, or overlap in time with each other. “Real-time” may refer to events that occur at distinct or non-overlapping times the difference between which is imperceptible and/or inconsequential to humans such as delays arising from electrical conduction or transmission. A human may perceive real-time events as occurring simultaneously, regardless of whether the real-time events occur at an exact same time. As a non-limiting example. “real-time” may refer to events that occur within a time frame of each other that is on the order of milliseconds, seconds, tens of seconds, or minutes. For example, “real-time” may refer to events that occur within a time frame of less than 1 minute, less than 30 seconds, less than 10 seconds, less than 1 second, less than 0.05 seconds, less than 0.01 seconds, less than 0.005 seconds, less than 0.001 seconds, etc.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example. “a processor configured to carry out recitations A. B and C” can include a first processor configured to carry out recitation A working in conjunction with a second processor configured to carry out recitations B and C.

As used herein. “system,” “instrument,” “apparatus,” and “device” generally encompass both the hardware (for example, mechanical and electronic) and, in some implementations, associated software (for example, specialized computer programs for operational control) components.

It should be emphasized that many variations and modifications may be made to the herein-described implementations, the elements of which are to be understood as being among other acceptable examples. All such modifications and variations are intended to be included herein within the scope of this disclosure. Any section headings used herein are merely provided to enhance readability and are not intended to limit the scope of the implementations disclosed in a particular section to the features or elements disclosed in that section. The foregoing description details certain implementations. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

1. An electronic device configured to be secured to a subject and measure one or more physiological parameters of the subject, the electronic device comprising:
a housing comprising:
a bottom portion configured to face toward tissue of the subject when the electronic device is secured to the subject's body;
a top portion opposite the bottom portion;
a first side connected to the top portion along a first edge of the housing;
a second side connected to the top portion along a second edge of the housing;
a third side connected to the top portion along a third edge of the housing;
a fourth side connected to the top portion along a fourth edge of the housing; and
an interior surface extending along the top portion, bottom portion, first side, second side, third side, and fourth side;
one or more physiological sensors for measuring the one or more physiological parameters of the subject; and
an antenna configured to allow the electronic device to wirelessly communicate with one or more separate devices, the antenna extending along the interior surface at least partially along each of the first edge, second edge, third edge, and fourth edge.

2. (canceled)

3. (canceled)

4. (canceled)

5. The electronic device of claim 1, wherein the antenna comprises a first antenna leg and a second antenna leg separated from one another.

6. The electronic device of claim 5, wherein:
the first antenna leg extends along said interior surface of the housing at least partially along the first edge, the third edge, and the fourth edge; and
the second antenna leg extends along said interior surface of the housing at least partially along the second edge, the third edge, and the fourth edge.

7. The electronic device of claim 5, wherein:
the first antenna leg comprises a first end and a second end;
the second antenna leg comprises a first end and a second end;
the first end of the first antenna leg is separated from the first end of the second antenna leg by a first gap; and
the second end of the first antenna leg is separated from the second end of the second antenna leg by a second gap.

8. The electronic device of claim 7, wherein:
the first ends of the first and second antenna legs are disposed along the fourth edge; and
the second ends of the first and second antenna legs are disposed along the third edge.

9. The electronic device of claim 7, wherein the first and second gaps are not aligned with one another.

10. The electronic device of claim 5, wherein the first antenna leg comprises a length and the second antenna leg comprises a length that is substantially equal to the length of the first antenna leg.

11. The electronic device of claim 5, wherein the first antenna leg extends along an entirety of the first edge and wherein the second antenna leg extends along an entirety of the second edge.

12. (canceled)

13. (canceled)

14. (canceled)

15. (canceled)

16. The electronic device of claim 1, wherein the first antenna leg comprises a uniform width between the first and second ends of the first antenna leg, and wherein the second antenna leg comprises a uniform width between the first and second ends of the second antenna leg.

17. The electronic device of claim 1, wherein the antenna is a dipole antenna.

18. The electronic device of claim 1, wherein:
a first corner of the housing is defined at an intersection of the first side and the fourth side;
a second corner of the housing is defined at an intersection of the fourth side and the second side;
a third corner of the housing is defined at an intersection of the second side and the third side;
a fourth corner of the housing is defined at an intersection of the third side and the first side;
the first antenna leg extends along the interior surface along the first and fourth corners; and
the second antenna leg extends along the interior surface along the second and third corners.

19. (canceled)

20. (canceled)

21. An electronic device comprising:
a housing comprising:
a bottom portion configured to face toward tissue of a subject when the electronic device is secured to a body of the subject;
a top portion opposite the bottom portion; and
an interior surface extending along the top portion;
a dipole antenna, configured to allow the electronic device to wirelessly communicate with one or more separate devices, wherein the dipole antenna is painted on the interior surface at least partially along the top portion.

22. (canceled)

23. (canceled)

24. (canceled)

25. The electronic device of claim 21, wherein the housing further comprises a first side connected to the top portion along a first edge of the housing, and wherein the dipole antenna is located along the interior surface along said first edge.

26. The electronic device of claim 21, wherein the dipole antenna is painted along the interior surface and spaced from a circuit board of the electronic device.

27. The electronic device of claim 21, wherein the dipole antenna is located on a corner of the interior surface of the top portion, wherein the dipole antenna extends along at least partially along a side of the interior surface.

28. An electronic device comprising:
a housing comprising:
a bottom portion configured to face toward tissue of a subject when the electronic device is secured to a body of the subject;
a top portion opposite the bottom portion;
an interior surface extending along the top portion;
an antenna comprising a first arm and a second arm, wherein the antenna is configured to allow the electronic device to wirelessly communicate with one or more separate devices;
a first contact pad coupled to the first arm of the antenna;
a second contact pad coupled to the second arm of the first arm of the antenna; and
a circuit board;
a first electrical connector extending between and contacting the circuit board and the first contact pad; and
a second electrical connector extending between and contacting the circuit board and the second contact pad.

29. The electronic device of claim 28, wherein the first electrical connector is configured to apply a force to the first contact pad and allow transfer of an electrical signal to the first arm, and wherein the second electrical connector is configured to apply a force to the second contact pad and allow transfer of the electrical signal to the second arm.

30. The electronic device of claim 29, wherein the first contact pad is configured to distribute the force from the first electrical connector and the second contact pad is configured to distribute the force from the second electrical connector.

31. The electronic device of claim 28, wherein the first contact pad is coupled to an end portion of the first arm.

32. (canceled)

33. The electronic device of claim 28, wherein the first contact pad and the second contact pad each comprise one or more metallic materials.

34. (canceled)

35. (canceled)
